(19) United States
(12) Patent Application Publication
(10) Pub. No.: US 2004/0048243 A1
(43) Pub. Date: Mar. 11, 2004

(54) Methods and compositions for in vitro targeting

(76) Inventors: Wadih Arap, Houston, TX (US); Renata Pasqualini, Houston, TX (US)

Correspondence Address: Blakely Sokoloff Taylor & Zafmann, Seventh Floor, 12400 Wilshire Boulevard, Los Angeles, CA, 90025, US

(21) Appl. No.: 10/363,208

(22) PCT Filed: Sep. 7, 2001
(86) PCT No.: PCT/US01/27702

(51) Int. Cl. C12Q 1/70; C12Q 1/68

(52) U.S. Cl. 435/5; 435/6

(57) ABSTRACT

The present invention concerns methods and comositions for in vivo and in vitro targeting. A large number of targeting peptides directed towards human organs, tissues or cell types are disclosed. The peptides are of use for targeted delivery of therapeutic agents, including but not limited to gene therapy vectors. A novel class of gene therapy vectors is disclosed. Certain of the disclosed peptides have therapeutic use for inhibiting angiogenesis, inhibiting tumor growth, inducing apoptosis, inhibiting pregnancy or inducing weight loss. Methods of identifying novel targeting peptides in humans, as well as identifying endogenous receptor-ligand pairs are disclosed. Methods of identifying novel infectious agents that are causal for human disease states are also disclosed. A novel mechanism for inducing apoptosis is further disclosed.

This application claims the benefit of U.S. Provisional Patent Application No. 60/231,266 filed Sep. 8, 2000, and U.S. patent application Ser. No. 09/765,101, filed Jan. 17, 2001. This invention was made with government support under grants DAMD 17-98-1-8041 and 17-98-1-8581 from the U.S. Army and grants 1R01CA78512-01A1, 1R1CA90810-01 and 1R01CA82976-01 from the National Institutes of Health. The government has certain rights in this invention.

### BACKGROUND OF THE INVENTION

1. Field of the Invention

The present invention concerns the fields of molecular medicine and targeted delivery of therapeutic agents. More specifically, the present invention relates to compositions and methods for the in vitro identification of peptides that selectively target organs, tissues or cell types and the therapeutic or diagnostic use of such peptides.

2. Description of Related Art

Therapeutic treatment of many disease states is limited by the systemic toxicity of the therapeutic agents used. Cancer therapeutic agents in particular exhibit a very low therapeutic index, with rapidly growing normal tissues such as skin and bone marrow affected at concentrations of agent that are not much higher than the concentrations used to kill tumor cells. Treatment of cancer and other organ, tissue or cell type confined disease states would be greatly facilitated by the development of compositions and methods for targeted delivery to a desired organ, tissue or cell type of a therapeutic agent.

Recently, an in vivo selection system was developed using phage display libraries to identify organ, tissue or cell type targeting peptides in a mouse model system. Phage display libraries expressing transgenic peptides on the surface of bacteriophage were initially developed to map epitope binding sites of immunoglobulins (Smith and Scott, 1986, 1993). Such libraries can be generated by inserting random oligonucleotides into cDNAs encoding a phage surface protein, generating collections of phage particles displaying unique peptides in as many as 10⁹ permutations. (Pasqualini and Ruoslahti, 1996, Arap et al, 1998a; Arap et al 1998b).

Intravenous administration of phage display libraries to mice was followed by the recovery of phage from individual organs (Pasqualini and Ruoslahti, 1996). Phage were recovered that were capable of selective homing to the vascular beds of different mouse organs, tissues or cell types, based on the specific targeting peptide sequences expressed on the outer surface of the phage (Pasqualini and Ruoslahti, 1996). A variety of organ and tumor-homing peptides have been identified by this method (Rajotte et al., 1998, 1999; Koivunen et al., 1999; Burg et al., 1999; Pasqualini, 1999). Each of those targeting peptides bound to different receptors that were selectively expressed on the vasculature of the mouse target tissue (Pasqualini, 1999; Pasqualini et al., 2000; Folkman, 1995; Folkman 1997). Tumor-homing peptides bound to receptors that were upregulated in the tumor angiogenic vasculature of mice (Brooks et al., 1994; Pasqualini et al., 2000). In addition to identifying individual targeting peptides selective for an organ, tissue or cell type (Pasqualini and Ruoslahti, 1996; Arap et al, 1998a; Koivunen et al., 1999), this system has been used to identify endothelial cell surface markers that are expressed in mice in vivo (Rajotte and Ruoslahti, 1999).

This relative success notwithstanding, cell surface selection of phage libraries has been plagued by technical difficulties. A high number of non-binder and non-specific binder clones are recovered using previous in vivo methods, particularly with components of the reticuloendothelial system such as spleen and liver. Removal of this background phage binding by repeated washes is both labor-intensive and inefficient. Cells and potential ligands are frequently lost during the many washing steps required. Thus, there is a need for rapid and efficient methods for in vitro phage display that retains the selectivity and specificity of in vivo methods, while providing decreased non-specific background.

Attachment of therapeutic agents to targeting peptides has resulted in the selective delivery of the agent to a desired organ, tissue or cell type in the mouse model system. Targeted delivery of chemotherapeutic agents and proapoptotic peptides to receptors located in tumor angiogenic vasculature resulted in a marked increase in therapeutic efficacy and a decrease in systemic toxicity in tumor-bearing mouse models (Arap et al., 1998a, 1998b; Ellerby et al., 1999).

Attempts have been made to target delivery of gene therapy vectors to specific organs, tissues or cell types in vivo. Directing such vectors to the site of interest would enhance therapeutic effects and diminish adverse systemic immunologic responses. Adenovirus type 5 (Ad5)-based vectors have been commonly used for gene transfer studies (Weitzman et al., 1997; Zhang, 1999). The attachment of Ad5 to the target cell is mediated by the capsid's fiber knob region, which interacts with cell surface receptors, including the coxsackie adenovirus receptor (CAR) and possibly with MHC class I (Bergelson et al., 1997; Hong et al., 1997). Upon systemic administration in vivo, binding of virus to CAR can result in unintended enrichment of vectors in non-targeted but CAR-expressing tissues. Conversely, target cells that express little or no CAR are inefficiently transduced. A need exists to develop novel gene therapy vectors to allow more selective delivery of gene therapy.

A need also exists to identify receptor-ligand pairs in organs, tissues or cell types. Previous attempts to identify targeted receptors and ligands binding to receptors have largely targeted a single ligand at a time for investigation. Identification of previously unknown receptors and previously uncharacterized ligands has been a very slow and laborious process. Such novel receptors and ligands may provide the basis for new therapies for a variety of disease states, such as diabetes mellitus, inflammatory disease, arthritis, atherosclerosis, cancer, autoimmune disease, bacterial infection, viral infection, cardiovascular disease or degenerative disease.

### SUMMARY OF THE INVENTION

The present invention solves a long-standing need in the art by providing compositions and in vitro methods for the identifying and using targeting peptides that are selective for specific organs, tissues or cell types. In certain embodiments, such methods and compositions may be used to identify one or more receptors for a targeting peptide. In alternative embodiments, the compositions and methods may be used to identify naturally occurring ligands for known or newly identified receptors.

In some embodiments, the methods may comprise contacting a targeting peptide to an organ, tissue or cell containing a receptor of interest, allowing the peptide to bind to the receptor, and identifying the receptor by its binding to the peptide. In preferred embodiments, the targeting peptide contains at least three contiguous amino acids selected from any of SEQ ID NO: 5 through SEQ ID NO: 65, SEQ ID NO: 67 through SEQ ID NO: 165 and SEQ ID NO: 176 through SEQ ID NO: 270. In other preferred embodiments, the targeting peptide comprises a portion of an antibody against the receptor. In alternative embodiments, the targeting peptide may contain a random amino acid sequence. The skilled artisan will realize that the contacting step can utilize intact organs, tissues or cells, or may alternatively utilize homogenates or detergent extracts of the organs, tissues or cells. In certain embodiments, the cells to be contacted may be genetically engineered to express a suspected receptor for the targeting peptide. In a preferred embodiment, the targeting peptide is modified with a reactive moiety that allows its covalent attachment to the receptor. In a more preferred embodiment, the reactive moiety is a photoreactive group that becomes covalently attached to the receptor when activated by light. In another preferred embodiment, the peptide is attached to a solid support and the receptor is purified by affinity chromatography. In other preferred embodiments, the solid support comprises magnetic beads, Sepharose beads, agarose beads, a nitrocellulose membrane, a nylon membrane, a column chromatography matrix, a high performance liquid chromatography (HPLC) matrix or a fast performance liquid chromatography (FPLC) matrix. In certain embodiments, the targeting peptide inhibits the activity of the receptor upon binding to the receptor. The skilled artisan will realize that receptor activity can be assayed by a variety of methods known in the art, including but not limited to catalytic activity and binding activity. In another preferred embodiment, the receptor is an endostatin receptor, a metalloprotease or an aminopeptidase.

In alternative embodiments, one or more ligands for a receptor of interest may be identified by the disclosed methods and compositions. One or more targeting peptides that mimic part or all of a naturally occurring ligand may be identified by phage display and biopanning. A naturally occurring ligand may be identified by homology with a single targeting peptide that binds to the receptor, or a consensus motif of sequences that bind to the receptor. In other alternative embodiments, an antibody may be prepared against one or more targeting peptides that bind to a receptor of interest. Such antibodies may be used for identification or immunoaffinity purification of the native ligand.

In certain embodiments, the targeting peptides of the present invention are of use for the selective delivery of therapeutic agents, including but not limited to gene therapy vectors and fusion proteins, to specific organs, tissues or cell types in subjects. The skilled artisan will realize that the scope of the claimed methods of use include any disease state that can be treated by targeted delivery of a therapeutic agent to a desired organ, tissue or cell type in a subject. Although such disease states include those where the diseased cells are confined to a specific organ, tissue or cell type, such as non-metastatic cancer, other disease states may be treated by an organ, tissue or cell type-targeting approach.

One embodiment of the present invention concerns isolated peptides of 100 amino acids or less in size, comprising at least 3 contiguous amino acids of a targeting peptide sequence, selected from any of SEQ ID NO: 5 through SEQ ID NO: 65, SEQ ID NO: 67 through SEQ ID NO: 165 and SEQ ID NO: 176 through SEQ ID NO: 270.

In a preferred embodiment, the isolated peptide is 50 amino acids or less, more preferably 30 amino acids or less, more preferably 20 amino acids or less, more preferably 10 amino acids or less, or even more preferably 5 amino acids or less in size. In other preferred embodiments, the isolated peptide of claim 1 comprises at least 4, 5, 6, 7, 8, 9, 10, 11, 12, 13, 14,15, 16, 17, 18, 19, 20, 21, 22, 23, 24 or 25 contiguous amino acids of a targeting peptide sequence, selected from any of SEQ ID NO: 5 through SEQ ID NO: 65, SEQ ID NO: 67 through SEQ ID NO: 165 and SEQ ID NO: 176 through SEQ ID NO: 270.

In certain embodiments, the isolated peptide is attached to a molecule. In preferred embodiments, the attachment is a covalent attachment. In additional embodiments, the molecule is a drug, a chemotherapeutic agent, a radioisotope, a pro-apoptosis agent, an anti-angiogenic agent, a hormone, a cytokine, a growth factor, a cytotoxic agent, a peptide, a protein, an antibiotic, an antibody, a Fab fragment of an antibody, an imaging agent, a survival factor, an anti-apoptotic agent, a hormone antagonist, a nucleic acid or an antigen. Those molecules are representative only. Molecules within the scope of the present invention include virtually any molecule that may be attached to a targeting peptide and administered to a subject. In preferred embodiments, the pro-aptoptosis agent is gramicidin, magainin, mellitin, defensin, cecropin, (KLAKLAK)₂ (SEQ ID NO: 1), (KLAKKLA)₂ (SEQ ID NO: 2), (KAAKKAA)₂ (SEQ ID NO: 3) or (KLGKKLG)₃ (SEQ ID NO: 4). In other preferred embodiments, the anti-angiogenic agent is angiostatin5, pigment epithelium-drived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP470, endostatin, paclitaxel, Docetaxel, polyamines, a proteasome inhibitor, a kinase inhibitor, a signaling peptide (Sugen, South San Francisco, Calif.), accutin, cidofovir, vincristine, bleomycin, AGM-1470, platelet factor 4 or minocycline. In further preferred embodiments, the cytokine is interleukin 1 (IL-1), IL-2, IL-5, IL-10, IL11, IL-12, IL-18, interferon-γ (IF-γ), IF-α, IF-β, tumor necrosis factor-α (TNF-α), or GM-CSF (granulocyte macrophage colony stimulating factor). Such examples are representative only and are not intended to exclude other pro-apoptosis agents, anti-angiogenic agents or cytokines known in the art.

In other embodiments, the isolated peptide is attached to a macromolecular complex. In preferred embodiments, the attachment is a covalent attachment. In other preferred embodiments, the macromolecular complex is a virus, a bacteriophage, a bacterium, a liposome, a microparticle, a magnetic bead, a yeast cell, a mammalian cell, a cell or a microdevice. These are representative examples only. Macromolecular complexes within the scope of the present invention include virtually any macromolecular complex that may be attached to a targeting peptide and administered to a subject. In other preferred embodiments, the isolated peptide is attached to a eukaryotic expression vector, more preferably a gene therapy vector.

In another embodiment, the isolated peptide is attached to a solid support, preferably magnetic beads, Sepharose beads, agarose beads, a nitrocellulose membrane, a nylon membrane, a column chromatography matrix, a high performance liquid chromatography (HPLC) matrix or a fast performance liquid chromatography (FPLC) matrix.

Additional embodiments of the present invention concern fusion proteins comprising at least 3 contiguous amino acids of a sequence selected from any of SEQ ID NO: 5 through SEQ ID NO: 65, SEQ ID NO: 67 through SEQ ID NO: 165 and SEQ ID NO: 176 through SEQ ID NO: 270.

Certain other embodiments concern compositions comprising the claimed isolated peptides or fusion proteins in a pharmaceutically acceptable carrier. Further embodiments concern kits comprising the claimed isolated peptides or fusion proteins in one or more containers.

Other embodiments concern methods of targeted delivery comprising selecting a targeting peptide for a desired organ, tissue or cell type, attaching said targeting peptide to a molecule, macromolecular complex or gene therapy vector, and providing said peptide attached to said molecule, complex or vector to a subject. Preferably, the targeting peptide is selected to include at least 3 contiguous amino acids from any of SEQ ID NO: 5 through SEQ ID NO: 65, SEQ ID NO: 67 through SEQ ID NO: 165 and SEQ ID NO: 176 through SEQ ID NO: 270. In certain preferred embodiments, the organ, tissue or cell type is bone marrow, lymph node, prostate cancer or prostate cancer that has metastasized to bone marrow. In other preferred embodiments, the molecule attached to the targeting peptide is a chemotherapeutic agent, an antigen or an imaging agent. The skilled artisan will realize that within the scope of the present invention any organ, tissue or cell type can be targeted for delivery, using targeting peptides attached to any molecule, macromolecular complex or gene therapy vector.

Other embodiments of the present invention concern isolated nucleic acids of 300 nucleotides or less in size, encoding a targeting peptide. In preferred embodiments, the isolated nucleic acid is 250, 225, 200, 175, 150, 125, 100, 75, 50, 40, 30, 20 or even 10 nucleotides or less in size. In other preferred embodiments, the isolated nucleic acid is incorporated into a eukaryotic or a prokaryotic expression vector. In even more preferred embodiments, the vector is a plasmid, a cosmid, a yeast artificial chromosome (YAC), a bacterial artificial chromosome (BAC), a virus or a bacteriophage. In other preferred embodiments, the isolated nucleic acid is operatively linked to a leader sequence that localizes the expressed peptide to the extracellular surface of a host cell.

Additional embodiments of the present invention concern methods of treating a disease state comprising selecting a targeting peptide that targets cells associated with the disease state, attaching one or more molecules effective to treat the disease state to the peptide, and administering the peptide to a subject with the disease state. Preferably, the targeting peptide includes at least three contiguous amino acids selected from any of SEQ ID NO: 5 through SEQ ID NO: 65, SEQ ID NO: 67 through SEQ ID NO: 165 and SEQ ID NO: 176 through SEQ ID NO: 270. In preferred embodiments the disease state is diabetes mellitus, inflammatory disease, arthritis, atherosclerosis, cancer, autoimmune disease, bacterial infection and viral infection.

Another embodiment of the present invention concerns molecular adaptors for targeted gene therapy in subjects. In a preferred embodiment, the molecular adaptor is a targeting peptide that has been incorporated into a surface protein of a gene therapy vector. In a more preferred embodiment, the gene therapy vector has been genetically engineered to contain AAV (adeno-associated virus) sequences. The skilled artisan will realize that the present invention is not limited as to the vector used, but may include any gene therapy vector that is known in the art. The only requirement is that the gene therapy vector should be selectively targeted to a desired organ, tissue or cell type in the presence of the molecular adaptor.

Another embodiment of the present invention concerns compositions and methods of use of tumor targeting peptides against cancers. Tumor targeting peptides identified by the methods disclosed in the instant application may be attached to therapeutic agents, including but not limited to molecules or macromolecular assemblages and administered to a patient with cancer, providing for increased efficacy and decreased systemic toxicity of the therapeutic agent. Therapeutic agents within the scope of the present invention include but are not limited to chemotherapeutic agents, radioisotopes, pro-apoptosis agents, cytotoxic agents, cytostatic agents and gene therapy vectors. Targeted delivery of such therapeutic agents to tumors provides a significant improvement over the prior art for increasing the delivery of the agent to the tumor, while decreasing the inadvertent delivery of the agent to normal organs and tissues of the patient. In a preferred embodiment, the tumor targeting peptide is incorporated into the capsule of a phage gene therapy vector to target delivery of the phage to angiogenic endothelial cells in tumor blood vessels.

Yet another embodiment of the present invention concerns methods of identifying targeting peptides against antibodies from an individual with a disease state, comprising obtaining a sample of serum from the individual, obtaining antibodies from the sample, adding a phage display library to the antibodies and collecting phage bound to the antibodies. In preferred embodiments, the antibodies are attached to a solid support, more preferably attached to protein G attached to beads. In another preferred embodiment, a subtraction step is added where the phage display library is first screened against antibodies from an individual who does not have the disease state. Only phage that do not bind to these control antibodies are used to obtain phage binding to the diseased individual's antibodies.

Certain embodiments concern methods of obtaining antibodies against an antigen. In preferred embodiments, the antigen comprises one or more targeting peptides. The targeting peptides are prepared and immobilized on a solid support, serum containing antibodies is added and antibodies that bind to the targeting peptides are collected.

In other preferred embodiments, a phage display library displaying the antigen binding portions of antibodies from a subject is prepared, the library is screened against one or more antigens and phage that bind to the antigens are collected. In more preferred embodiments, the antigen is a targeting peptide.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Selection of peptide library on immunoglobulins from the serum of metastatic prostate cancer patients. Each successive round of panning demonstrates an increase in selectivity as measured by the increase in total number of transducing units for cancer patients relative to the serum of control volunteers. Three metastatic androgen-independents (patients A, B, and D) serum samples and one metastatic androgen-dependent (patient C) serum sample were examined. Standard error of the mean (S.E.M.) from triplicate plating is shown.

FIG. 2. Selection of peptide library on immunoglobulins from the serum of metastatic prostate cancer patients. A series of 100-fold dilutions (1:100-1:1200) was performed for each patient's serum to test specific binding of cancer antibodies to immobilized GST-fusion proteins by ELISA.

FIG. 3A. Correlation between patients' survival outcome and peptides' reactivity according to the Kaplan-Meier method. The data represents all of the prostate cancer patients examined. A log-rank test was implemented to detect significant difference in survival time between peptide reacting group versus non-reacting group. No statistical significance was observed with peptide A (p-value of 0.75).

FIG. 3B. Correlation between patients' survival outcome and peptides' reactivity according to the Kaplan-Meier method. No statistical significance was observed with peptide B (p-value of 0.83).

FIG. 3C. Correlation between patients' survival outcome and peptides' reactivity according to the Kaplan-Meier method. A strong statistical correlation existed between patient survival outcome and peptide C reactivity (p-value=0.04). P-values less than 0.05 were considered significant.

FIGS. 4A-4F. A homology search was run of the phage peptide sequences binding to endostatin versus known protein databases. Candidate proteins for endogenous receptors for endostatin are indicated, along with the degree of homology and, where available, the GenBank accession number.

FIG. 5. Panning of phage peptide library on rhAngiostatin. The rhAngiostatin protein was immobilized onto microtiter wells and screened against a CX₇C library. Bound phage were recovered after infection with K91 bacteria. Results illustrate the average number of phage—transducing units ×10² recovered per well when coated with rhAngiostatin, rh-Endostatin and BSA after the first, second and third rounds of selection. (First round to left, second round in middle, and third round to right of FIG. 20).

FIG. 6. Selectivity of rhAngiostatin-targeted phage to immobilized proteins. rhAngiostatin, rh-Endostatin and rmEndostatin and BSA were coated on microtiter wells at 1 μg/ml and used to bind phage expressing the selected angiostatin targetting peptides. The data represent the mean colony counts from triplicate wells, with standard error less than 10% of the mean.

FIG. 7. CD13 and HGF bind to rhAngiostatin. The wells of a 96-well plate were coated with 1 μg/ml of protein (CD13, HGF, TSP-1, LN, COL IV, FN, VN or BSA), and incubated with rhAngiostatin. The amount of rhAngiostatin binding to each well was determined spectrophotometrically following the addition of anti-hAngiostatin polyclonal antibody, peroxidase conjugated anti-goat IgG and substrate. Error bars indicate standard error of the mean.

FIG. 8. Selection of a peptide library on immunoglobulins from serum of a patient with Hodgkin's disease. Each successive round of panning show increased specificity since the number of IgG bound infective phage, as measured by the total number of transducing units, increases for the HD sera relative to the control sera.

FIG. 9. Selected peptide motifs and homologous proteins. Targeting peptides against circulating antibodies from the sera of Hodgkin's disease patients were identified as described. Homologous proteins were identified by computer search of the Swiss Protein database.

FIG. 10. Sequence homologies between HD targeting peptides and viral proteins from Macaca mulatta rhadinovirus, KSHV (Kaposi's sarcoma-associated virus) and EBV (Epstein Barr virus).

FIG. 11A. Transduction of tumor cells by targeted phage is specific. Quantitative analysis of cell transduction by targeted and control phage. Tumor cells were incubated with targeted (HWGF-β-gal or RGD-4C-β-gal) or control insertless phage (fd-tet-β-gal). An anti-β-gal antibody was used for staining; gene expression was detected by immunofluorescence and results are expressed in % of β-gal positive cells. In each case, standard error of the mean (SEM) was calculated after counting 10 fields under the microscope in three independent experiments.

FIG. 11B. Transduction of tumor cells by targeted phage is specific. Inhibition of HWGF-β-gal phage transduction by the synthetic CTTHWGFTLC (SEQ ID NO: 167) peptide. Control peptides did not inhibit transduction of the tumor cells by the targeted phage. Non-specific transduction levels were determined by using the control insertless phage. Shown are mean±SEM obtained from duplicate wells.

FIG. 11C. Transduction of tumor cells by targeted phage is specific. Inhibition of RGD-4C-β-gal phage transduction by the synthetic RGD-4C peptide (SEQ ID NO: 166). Control peptides did not inhibit transduction of the tumor cells by the targeted phage. Non-specific transduction levels were determined by using the control insertless phage. Shown are mean±SEM obtained from duplicate wells.

FIG. 12. Specific transduction in vivo by lung-targeting phage. Lung (targeted organ) and liver (control organ) were evaluated for β-gal expression after systemic administration of lung targeting GFE-phage or control phage into C57B1/6 immunocompetent mice. At 14 days post-administration lungs and livers were removed and processed. β-gal enzymatic activity in the tissue cell lysates was measured by chemiluminescence. Shown are mean±SEM (n=5 mice per group).

FIG. 13. Enhancement of transduction by genotoxic agents or genetic trans-complementation. Semi-confluent cells were infected with 10⁵ TU of phage per cell for four hours. Next, the cells were incubated for 36 hours followed by addition of genotoxic drugs (topotecan, 10 μM; cisplatin, 10 μM) or application of physical agents such as ultraviolet radiation (UV; 15 J/m²). A phage mixture of RGD-4-βgal forward and reverse clones (molar ratio=1; termed For/Rev) at the same number of phage TU of RGD4C-βgal phage was also tested. At 72 hours post-infection, the cells were analyzed for expression of a reporter transgene. Shown are mean±SEM (n=3) normalized green fluorescent protein (GFP) expression relative to controls.

FIG. 14. Quantification of β-galactosidase expression in target cells after transduction with RGD-4C-β-gal phage particles. RGD4C-β-gal phage were incubated with MDA-MB435 cells and HWGF-β-gal phage were incubated with KS1767 cells. The untargeted phage vector fd-β-gal was used as a control. β-galactosidase expression was assessed by immunofluorescence and counting positive cells.

FIG. 15. Cloning strategy. A two-step cloning strategy was employed. Replicating forms (RFs) of the RGD4C DNA (9.5 kb) and of fMCS1 DNA (4.1 kb) were prepared. A chimeric RGD4C-fMCS1 phage vector was obtained by subcloning the 5.4 kb Bam HI/Sac II fragment of RF RGD4C into the Bam HI/Sac II sites of fMCS1. The fMCS1 vector is a fd-tet derived phage vector not used for display, but which contains a multiple cloning site (MCS) including a PstI site. RGD4C-fMCS1 retains the ability to display the RGD4C peptide on the gene III protein and carries a MCS. The final RGD4C-βgal phage construct (14 kb) was obtained by subcloning a PstI fragment containing a 4.5 kb CMV-driven eukaryotic βgal cassette into RGD4C-fMCS1 in forward and reverse orientations. The resulting phage vectors were termed fRGD4C-βgal and rRGD4C-βGal.

FIG. 16. Gene expression of transgenic phage in eukaryotic cells. Uptake and expression of phage encoding a marker gene was examined in human cells.

FIG. 17. Protocol for recovery of phage by infection of E. coli or recovery of phage DNA by amplification and subcloning.

FIG. 18. Pancreatic islet targeting peptides and homologous proteins. Candidate endogenous proteins mimicked by the pancreatic islet targeting peptides CVSNPRWKC (SEQ ID NO: 236), CVPRRWDVC (SEQ ID NO: 233), CQHTSGRGC (SEQ ID NO: 234) and CRARGWLLC (SEQ ID NO: 235), identified by standard homology searches.

FIG. 19. Pancreatic islet targeting peptides and homologous proteins. Candidate endogenous proteins mimicked by the pancreatic islet targeting peptides CGGVHALRC (SEQ ID NO: 220), CFNRTWIGC (SEQ ID NO: 237) and CWSRGQGGC (SEQ ID NO: 239), identified by standard homology searches.

FIG. 20. Pancreatic islet targeting peptides and homologous proteins. Candidate endogenous proteins mimicked by the pancreatic islet targeting peptides CLASGMDAC (SEQ ID NO: 243), CHDERTGRC (SEQ ID NO: 244), CAHHALMEC (SEQ ID NO: 245) and CMQGAATSC (SEQ ID NO: 246), identified by standard homology searches.

FIG. 21. Pancreatic islet targeting peptides and homologous proteins. Candidate endogenous proteins mimicked by the pancreatic islet targeting peptides CHVLWSTRC (SEQ ID NO: 240), CMSSPGVAC (SEQ ID NO: 242) and CLGLLMAGC (SEQ ID NO: 241), identified by standard homology searches.

FIG. 22. Binding of phage containing the CVPELGHEC (SEQ ID NO: 271) and CFELGFELGC (SEQ ID NO: 272) targeting peptides to IgG's isolated from ovarian cancer patient #2 ascites, normal serum and BSA.

FIG. 23. Binding of phage containing the CVPELGHEC (SEQ ID NO: 271) targeting peptide to IgG's isolated from normal serum or from ascites of ovarian cancer patient #1 or #2. Control fd-tet phage contained no insert DNA.

FIG. 24. Binding of phage containing the CVPELGHEC (SEQ ID NO: 271) targeting peptide to IgG's isolated from serum of ovarian cancer patient #2, normal serum or BSA.

FIG. 25. Homology between ovarian cancer targeting peptides ELGFELG (SEQ ID NO: 250) and VPELGHE (SEQ ID NO: 249) to matrix metalloproteinase proteins.

### DESCRIPTION OF ILLUSTRATIVE EMBODIMENTS

As used herein in the specification, “a” or “an” may mean one or more. As used herein in the claim(s), in conjunction with the word “comprising,” the words “a” or “an” may mean one or more than one. As used herein “another” may mean at least a second or more of an item.

A “targeting peptide” is a peptide comprising a contiguous sequence of amino acids, that is characterized by selective localization to an organ, tissue or cell type. Selective localization may be determined, for example, by methods disclosed below, wherein the putative targeting peptide sequence is incorporated into a protein that is displayed on the outer surface of a phage. A phage expressing a targeting peptide sequence is considered to be selectively locallized to a cell type, tissue or organ if it exhibits greater binding in that tissue or organ compared to a control tissue or organ. Preferably, selective localization of a targeting peptide should result in a two-fold or higher enrichment of the phage in the target organ, tissue or cell type, compared to a control organ, tissue or cell type. An alternative method to determine selective localization is that phage expressing the putative target peptide preferably exhibit a two-fold, more preferably a three-fold or higher enrichment in the target organ compared to control phage that express a non-specific peptide or that have not been genetically engineered to express any putative target peptides. Another method to determine selective localization is that locallization to the target organ, tissue or cell type of phage expressing the target peptide is at least partially blocked by the co-administration of a synthetic peptide containing the target peptide sequence. “Targeting peptide” and “homing peptide” are used synonymously herein.

A “phage display library” means a collection of phage that have been genetically engineered to express a set of putative targeting peptides on their outer surface. In preferred embodiments, DNA sequences encoding the putative targeting peptides are inserted in frame into a gene encoding a phage capsule protein. In other preferred embodiments, the putative targeting peptide sequences are in part random mixtures of all twenty amino acids and in part non-random. In certain preferred embodiments the putative targeting peptides of the phage display library exhibit one or more cysteine residues at fixed locations within the targeting peptide sequence.

A “macromolecular complex” refers to a collection of molecules that may be random, ordered or partially ordered in their arrangement. The term encompasses biological organisms such as bacteriophage, viruses, bacteria, unicellular pathogenic organisms, multicellular pathogenic organisms, and prokaryotic or eukaryotic cells. The term also encompasses non-living assemblages of molecules, such as liposomes, microcapsules, microparticles, magnetic beads and microdevices. The only requirement is that the complex contains more than one molecule. The molecules may be identical, or may differ from each other.

A “receptor” for a targeting peptide includes but is not limited to any molecule or complex of molecules that binds to a targeting peptide. Non-limiting examples of receptors include peptides, proteins, glycoproteins, lipoproteins, epitopes, lipids, carbohydrates, multi-molecular structures, a specific conformation of one or more molecules and a morphoanatomic entity. In preferred embodiments, a “receptor” is a naturally occurring molecule or complex of molecules that is present on the lumenal surface of cells forming blood vessels within a target organ, tissue or cell type.

A “subject” refers generally to a mammal. In certain preferred embodiments, the subject is a mouse or rabbit. In even more preferred embodiments, the subject is a human.

Phage Display

The methods described herein for identification of targeting peptides involve the in vitro administration of phage display libraries. Various methods of phage display and methods for producing diverse populations of peptides are well known in the art. For example, U.S. Pat. Nos. 5,223,409; 5,622,699 and 6,068,829, each of which is incorporated herein by reference, disclose methods for preparing a phage library. The phage display technique involves genetically manipulating bacteriophage so that small peptides can be expressed on their surface (Smith et al., 1985, 1993). The potential range of applications for this technique is quite broad, and the past decade has seen considerable progress in the construction of phage-displayed peptide libraries and in the development of screening methods in which the libraries are used to isolate peptide ligands. For example, the use of peptide libraries has made it possible to characterize interacting sites and receptor-ligand binding motifs within many proteins, such as antibodies involved in inflammatory reactions or integrins that mediate cellular adherence. This method has also been used to identify novel peptide ligands that serve as leads to the development of peptidomimetic drugs or imaging agents (Arap et al., 1998a). In addition to peptides, larger protein domains such as single-chain antibodies can also be displayed on the surface of phage particles (Arap et al., 1998a).

Previous methods for identifying amino acid sequences for a targeting a given organ, tissue or cell type involved isolation by “biopanning” (Pasqualini and Ruoslahti, 1996; Pasqualini, 1999). A library of phage containing putative targeting peptides was administered to an animal model and samples of organs, tissues or cell types containing phage were collected.

In either in vivo or in vitro methods utilizing filamentous phage, the phage may be recovered from a sample of organ, tissue or cell type that has been exposed to a phage library. In alternative embodiments, phage may be recovered by infecting them into pilus-positive bacteria. The bacteria are not lysed by the phage but rather secrete multiple copies of phage that display a particular insert. Phage that bind to a target can be eluted from the organ, tissue or cell type and amplified by growing them in host bacteria. Alternatively, targeting peptide sequences may be amplified from the sample and inserted into fresh phage DNA, then infected into host bacteria. By either technique, targeting peptides may be recovered from the target and amplified.

The amino acid sequence of the peptides may be determined by sequencing the DNA corresponding to the targeting peptide insert in the phage genome. The identified targeting peptide can then be produced as a synthetic peptide by standard protein chemistry techniques (Arap et al., 1998a, Smith et al., 1985). This approach allows circulating targeting peptides to be detected in an unbiased functional assay, without any preconceived notions about the nature of their target. Once a candidate target is identified as the receptor of a targeting peptide, it can be isolated, purified and cloned by using standard biochemical methods (Pasqualini, 1999; Rajotte and Ruoslahti, 1999).

Choice of Phage Display System.

Previous in vivo selection studies performed in mice preferentially employed libraries of random peptides expressed as fusion proteins with the gene III capsule protein in the fUSE5 vector (Pasqualini and Ruoslahti, 1996). The number and diversity of individual clones present in a given library is a significant factor for the success of in vivo selection. It is preferred to use primary libraries, which are less likely to have an over-representation of defective phage clones (Koivunen et al., 1999). The preparation of a library should be optimized to between 10⁸-10⁹ transducing units (T.U.)/ml. In certain embodiments, a bulk amplification strategy is applied between each round of selection.

Phage libraries displaying linear, cyclic, or double cyclic peptides may be used within the scope of the present invention. However, phage libraries displaying 3 to 10 random residues in a cyclic insert (CX₃₋₁₀C) are preferred, since single cyclic peptides tend to have a higher affinity for the target organ than linear peptides. Libraries displaying double-cyclic peptides (such as CX₃C X₃CX₃C; Rojotte et al., 1998) have been successfully used. However, the production of the cognate synthetic peptides, although possible, can be complex due to the multiple conformers with different dissulfide bridge arrangements.

Identification of Homing Peptides and Receptors by in vivo Phage Display in Mice.

In vivo selection of peptides from phage-display peptide libraries administered to mice has been used to identify targeting peptides selective for normal mouse brain, kidney, lung, skin, pancreas, retina, intestine, uterus, prostate, and adrenal gland (Pasqualini and Ruoslahti, 1996; Pasqualini, 1999; Rajotte et al., 1998). These results show that the vascular endothelium of normal organs is sufficiently heterogenous to allow differential targeting with peptide probes (Pasqualini and Ruoslahti, 1996; Rajotte et al., 1998). A means of identifying peptides that home to the angiogenic vasculature of tumors has been devised, as described below. A panel of peptide motifs that target the blood vessels of tumor xenografts in nude mice has been assembled (Arap et al., 1998a; reviewed in Pasqualini, 1999). These motifs include the sequences RGD-4C, NGR, and GSL. The RGD-4C peptide has previously been identified as selectively binding αv integrins and has been shown to home to the vasculature of tumor xenografts in nude mice (Arap et al., 1998a, 1998b; Pasqualini et al., 1997).

The receptors for the tumor homing RGD4C targeting peptide has been identified as αv integrins (Pasqualini et al., 1997). The αv integrins play an important role in angiogenesis. The αvβ3 and αvβ5 integrins are absent or expressed at low levels in normal endothelial cells but are induced in angiogenic vasculature of tumors (Brooks et al., 1994; Hammes et al., 1996). Aminopeptidase N/CD13 has recently been identified as an angiogenic receptor for the NGR motif (Burg et al., 1999). Aminopeptidase N/CD13 is strongly expressed in the angiogenic blood vessels of cancer and in other angiogenic tissues.

Tumor-homing phage co-localize with their receptors in the angiogenic vasculature of tumors but not in non-angiogenic blood vessels in normal tissues (Arap et al., 1998b). Immunohistochemical evidence shows that vascular targeting phage bind to human tumor blood vessels in tissue sections (Pasqualini et al., 2000) but not to normal blood vessels. A negative control phage with no insert (fd phage) did not bind to normal or tumor tissue sections. The expression of the angiogenic receptors was evaluated in cell lines, in non-proliferating blood vessels and in activated blood vessels of tumors and other angiogenic tissues such as corpus luteum. Flow cytometry and immunohistochemistry showed that these receptors are expressed in a number of tumor cells and in activated HUVECs (data not shown). The angiogenic receptors were not detected in the vasculature of normal organs of mouse or human tissues.

The distribution of these receptors was analyzed by immunohistochemistry in tumor cells, tumor vasculature, and normal vasculature. Alpha v integrins, CD13, aminopeptidase A, NG2, and MMP-2/MMP-9—the known receptors in tumor blood vessels—are specifically expressed in angiogenic endothelial cells and pericytes of both human and murine origin. Angiogenic neovasculature expresses markers that are either expressed at very low levels or not at all in non-proliferating endothelial cells (not shown).

The markers of angiogenic endothelium include receptors for vascular growth factors, such as specific subtypes of VEGF and basic FGF receptors, and αv integrins, among many others (Mustonen and Alitalo, 1995). Thus far, identification and isolation of novel molecules characteristic of angiogenic vasculature has been slow, mainly because endothelial cells undergo dramatic phenotypic changes when grown in culture (Watson et al., 1995).

Many of these tumor vascular markers are proteases and some of the markers also serve as viral receptors. Alpha v integrins are receptors for adenoviruses (Wickham et al., 1997c) and CD13 is a receptor for coronaviruses (Look et al., 1989). MMP-2 and MMP-9 are receptors for echoviruses (Koivunen et al., 1999). Aminopeptidase A also appears to be a viral receptor. Bacteriophage may use the same cellular receptors as eukaryotic viruses. These findings suggest that receptors isolated by in vivo phage display will have cell internalization capability, a key feature for utilizing the identified peptide motifs as targeted gene therapy carriers.

Targeted Delivery

Peptides that home to tumor vasculature have been coupled to cytotoxic drugs or proapoptotic peptides to yield compounds that were more effective and less toxic than the parental compounds in experimental models of mice bearing tumor xenografts (Arap et al., 1998a; Ellerby et al, 1999). As described below, the insertion of the RGD-4C peptide into a surface protein of an adenovirus has produced an adenoviral vector that may be used for tumor targeted gene therapy (Arap et al., 1998b).

Brasil

In preferred embodiments, separation of phage bound to the cells of a target organ, tissue or cell type from unbound phage is achieved using the BRASIL technique (Provisional Patent Application No. 60/231,266 filed Sep. 8, 2000; U.S. patent application entitled, “Biopanning and Rapid Analysis of Selective Interactive Ligands (BRASIL)” by Arap, Pasqualini and Giordano, filed concurrently herewith, incorporated herein by reference in its entirety). In BRASIL (Biopanning and Rapid Analysis of Soluble Interactive Ligands), an organ, tissue or cell type is gently separated into cells or small clumps of cells that are suspended in an first, preferably aqueous phase. The aqueous phase is layered over a second, preferably organic phase of appropriate density and centrifuged. Cells attached to bound phage are pelleted at the bottom of the centrifuge tube, while unbound phage remain in the aqueous phase. This allows a more efficient separation of bound from unbound phage, while maintaining the binding interaction between phage and cell. BRASIL may be performed in an in vitro protocol, in which organs, tissues or cell types are exposed to a phage display library in an aqueous phase in vitro before centrifugation.

In certain embodiments, a subtraction protocol may be used with BRASIL or other screening protocols to further reduce background phage binding. The purpose of subtraction is to remove phage from the library that bind to cells other than the cell of interest, or that bind to inactivated cells. In alternative embodiments, the phage library may be screened against a control cell line, tissue or organ sample that is not the targeted cell, tissue or organ. After subtraction the library may be screened against the cell, tissue or organ of interest. In another alternative embodiment, an unstimulated, quiescent cell line, tissue or organ may be screened against the library and binding phage removed. The cell line, tissue or organ is then activated, for example by administration of a hormone, growth factor, cytokine or chemokine and the activated cell line screened against the subtracted phage library.

Other methods of subtraction protocols are known and may be used in the practice of the present invention, for example as disclosed in U.S. Pat. Nos. 5,840,841, 5,705,610, 5,670,312 and 5,492,807, incorporated herein by reference.

Proteins and Peptides

In certain embodiments, the present invention concerns novel compositions comprising at least one protein or peptide. As used herein, a protein or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide” and “peptide are used interchangeably herein.

In certain embodiments the size of the at least one protein or peptide may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues.

As used herein, an “amino acid residue” refers to any naturally occuring amino acid, any amino acid derivitive or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid, including but not limited to those shown on Table 1 below.

TABLE 1
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino- | AHyl | allo-Hydroxylysine |
| | propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine. | | |

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for known genes may be amplified and/or expressed using the techniques disclosed herein or as would be know to those of ordinary skill in the art. Alternatively, various commercial preparations of proteins, polypeptides and peptides are known to those of skill in the art.

Peptide Mimetics

Another embodiment for the preparation of polypeptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993), incorporated herein by reference. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting peptides disclosed herein, but with altered and even improved characteristics.

Fusion Proteins

Other embodiments of the present invention concern fusion proteins. These molecules generally have all or a substantial portion of a targeting peptide, linked at the N- or C-terminus, to all or a portion of a second polypeptide or proteion. For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In preferred embodiments, the fusion proteins of the instant invention comprise a targeting peptide linked to a therapeutic protein or peptide. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. These examples are not meant to be limiting and it is contemplated that within the scope of the present invention virtually and protein or peptide could be incorporated into a fusion protein comprising a targeting peptide. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

Protein Purification

In certain embodiments a protein or peptide may be isolated or purified. Protein purification techniques are well known to those of skill in the art. These techniques involve, at one level, the homogenization and crude fractionation of the cells, tissue or organ to polypeptide and non-polypeptide fractions. The protein or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. An example of receptor protein purification by affinity chromatography is disclosed in U.S. Pat. No. 5,206,347, the entire text of which is incorporated herein by reference. A particularly efficient method of purifying peptides is fast protein liquid chromatography (FPLC) or even HPLC.

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind to. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand.

Synthetic Peptides

Because of their relatively small size, the targeting peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, usually from about 6 up to about 35 to 50 amino acids, can be readily synthesized by such methods. Alternatively, recombinant DNA technology may be employed wherein a nucleotide sequence which encodes a peptide of the invention is inserted into an expression vector, transformed or transfected into an appropriate host cell, and cultivated under conditions suitable for expression.

Antibodies

In certain embodiments, it may be desirable to make antibodies against the identified targeting peptides or their receptors. The appropriate targeting peptide or receptor, or portions thereof, may be coupled, bonded, bound, conjugated, or chemically-linked to one or more agents via linkers, polylinkers, or derivatized amino acids. This may be performed such that a bispecific or multivalent composition or vaccine is produced. It is further envisioned that the methods used in the preparation of these compositions are familiar to those of skill in the art and should be suitable for administration to humans, i.e., pharmaceutically acceptable. Preferred agents are the carriers are keyhole limpet hemocyanin (KLH) or bovine serum albumin (BSA).

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Cytokines and Chemokines

In certain embodiments, it may be desirable to couple specific bioactive agents to one or more targeting peptides for targeted delivery to an organ, tissue or cell type. Such agents include, but are not limited to, cytokines, chemikines, pro-apoptosis factors and anti-angiogenic factors. The term “cytokine” is a generic term for proteins released by one cell population which act on another cell as intercellular mediators. Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-.alpha. and -.beta.; mullerian-inhibiting substance; mouse gonadotropin-associated peptide; inhibin; activin; vascular endothelial growth factor; integrin; thrombopoietin (TPO); nerve growth factors such as NGF-.beta.; platelet-growth factor; transforming growth factors (TGFs) such as TGF-.alpha. and TGF-.beta.; insulin-like growth factor-I and -II; erythropoietin (EPO); osteoinductive factors; interferons such as interferon-α, -.β, and -γ; colony stimulating factors (CSFs) such as macrophage-CSF (M-CSF); granulocyte-macrophage-CSF (GM-CSF); and granulocyte-CSF (G-CSF); initerleukins (ILs) such as IL-1, IL-1.alpha., IL-2, IL-3, IL4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand or FLT-3, angiostatin, thrombospondin, endostatin, tumor necrosis factor and LT. As used herein, the term cytokine includes proteins from natural sources or from recombinant cell culture and biologically active equivalents of the native sequence cytokines.

Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Chemokines include, but are not limited to, RANTES, MCAF, MIP1-alpha, MIP1-Beta, and IP-10. The skilled artisan will recognize that certain cytokines are also known to have chemoattractant effects and could also be classified under the term chemokines.

Imaging Agents and Radioisotopes

In certain embodiments, the claimed peptides or proteins of the present invention may be attached to imaging agents of use for imaging and diagnosis of various diseased organs, tissues or cell types. Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

Radioisotopes of potential use as imaging or therapeutic agents include astatine²¹¹, ¹⁴carbon, ^{5l}chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being preferred for use in certain embodiments, and technicium^{99m} and indium¹¹¹ are also often preferred due to their low energy and suitability for long range detection.

Radioactively labeled proteins or peptides of the present invention may be produced according to well-known methods in the art. For instance, they can be iodinated by contact with sodium or potassium iodide and a chemical oxidizing agent such as sodium hypochlorite, or an enzymatic oxidizing agent, such as lactoperoxidase. Proteins or peptides according to the invention may be labeled with technetium-^{99m} by ligand exchange process, for example, by reducing pertechnate with stannous solution, chelating the reduced technetium onto a Sephadex column and applying the peptide to this column or by direct labeling techniques, e.g., by incubating pertechnate, a reducing agent such as SNCl₂, a buffer solution such as sodium-potassium phthalate solution, and the peptide. Intermediary functional groups which are often used to bind radioisotopes which exist as metallic ions to peptides are diethylenetriaminepentaacetic acid (DTPA) and ethylene diaminetetracetic acid (EDTA). Also contemplated for use are fluorescent labels, including rhodamine, fluorescein isothiocyanate and renographin.

In certain embodiments, the claimed proteins or peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

Cross-Linkers

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-lining between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites are dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups.

Nucleic Acids

Nucleic acids according to the present invention may encode a targeting peptide, a receptor protein or a fusion protein. The nucleic acid may be derived from genomic DNA, complementary DNA (cDNA) or synthetic DNA. Where incorporation into an expression vector is desired, the nucleic acid may also comprise a natural intron or an intron derived from another gene. Such engineered molecules are sometime referred to as “mini-genes.”

A “nucleic acid” as used herein includes single-stranded and double-stranded molecules, as well as DNA, RNA, chemically modified nucleic acids and nucleic acid analogs. It is contemplated that a nucleic acid within the scope of the present invention maybe of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater nucleotide residues in length.

It is contemplated that targeting peptides, fusion proteins and receptors may be encoded by any nucleic acid sequence that encodes the appropriate amino acid sequence. The design and production of nucleic acids encoding a desired amino acid sequence is well known to those of skill in the art, using standardized codon tables (see Table 2 below). In preferred embodiments, the codons selected for encoding each amino acid may be modified to optimize expression of the nucleic acid in the host cell of interest. Codon preferences for various species of host cell are well known in the art.

TABLE 2

| Amino Acid | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In addition to nucleic acids encoding the desired targeting peptide, fusion protein or receptor amino acid sequence, the present invention encompasses complementary nucleic acids that hybridize under high stringency conditions with such coding nucleic acid sequences. High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture.

Vectors for Cloning, Gene Transfer and Expression

In certain embodiments expression vectors are employed to express the targeting peptide or fusion protein, which can then be purified and used. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are known.

Regulatory Elements

The terms “expression construct” or “expression vector” are meant to include any type of genetic construct containing a nucleic acid coding for a gene product in which part or all of the nucleic acid coding sequence is capable of being transcribed. In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, rat insulin promoter, and glyceraldehyde-3-phosphate dehydrogenase promoter can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

Where a cDNA insert is employed, typically one will typically include a polyadenylation signal to effect proper polyadenylation of the gene transcript. The nature of the polyadenylation signal is not believed to be crucial to the successful practice of the invention, and any such sequence may be employed, such as human growth hormone and SV40 polyadenylation signals. Also contemplated as an element of the expression construct is a terminator. These elements can serve to enhance message levels and to minimize read through from the construct into other sequences.

Selectable Markers

In certain embodiments of the invention, the cells containing nucleic acid constructs of the present invention may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin, and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome, and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Preferred gene therapy vectors are generally viral vectors.

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression making them ideally suited for rapid, efficient, heterologous gene expression. Techniques for preeparing replication infective viruses are well known in the art.

In using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

DNA viruses used as gene vectors include the papovaviruses (e.g., simian virus 40, bovine papilloma virus, and polyoma) (Ridgeway, 1988; Baichwal and Sugden, 1986) and adenoviruses (Ridgeway, 1988; Baichwal and Sugden, 1986). In a preferred embodiment, disclosed in the following Examples, filamentous bacteriophage expressing targeting peptides in a surface protein may be genetically engineered to contain elements of viral sequences, such as AAV sequences, and used as targeted expression vectors.

One of the preferred methods for in vivo delivery involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include, but is not limited to, constructs containing adenovirus sequences sufficient to (a) support packaging of the construct and (b) to express an antisense or a sense polynucleotide that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retroviral infection, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

In currently used systems, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of adenovirus vectors which are replication deficient depend on a unique helper cell line, designated 293, which is transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3, or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As discussed, the preferred helper cell line is 293.

Racher et al., (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) are employed as follows. A cell innoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking is initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking is commenced for another 72 hr.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

A typical vector applicable to practicing the present invention is replication defective and will not have an adenovirus E1 region. Thus, it is most convenient to introduce the polynucleotide encoding the gene at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors as described by Karlsson et al., (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic innoculation into the brain (Le Gal La Salle et al., 1993).

Other gene transfer vectors may be constructed from retroviruses. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env. that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences, and also are required for integration in the host cell genome (Coffin, 1990).

In order to construct a retroviral vector, a nucleic acid encoding protein of interest is inserted into the viral genome in the place of certain viral sequences to produce a virus that is replication-defective. In order to produce virions, a packaging cell line containing the gag, pol, and env genes, but without the LTR and packaging components, is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are capable of infecting a broad variety of cell types. However, integration and stable expression require the division of host cells (Paskind et al., 1975).

There are certain limitations to the use of retrovirus vectors. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This may result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs. Vectors derived from viruses such as vaccinia virus (Ridgeway, 1988; Baichwal and Sugden, 1986; Coupar et al., 1988), adeno-associated virus (AAV) (Ridgeway, 1988; Baichwal and Sugden, 1986; Hermonat and Muzycska, 1984), and herpes viruses may be employed. They offer several attractive features for various mammalian cells (Friedmann, 1989; Ridgeway, 1988; Baichwal and Sugden, 1986; Coupar et al., 1988; Horwich et al., 1990).

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990), DEAE-dextran (Gopal, 1985), electroporation (Tur-Kaspa et al., 1986; Potter et al., 1984), direct microinjection, DNA-loaded liposomes and lipofectamine-DNA complexes, cell sonication, gene bombardment using high velocity microprojectiles, and receptor-mediated transfection (Wu and Wu, 1987; Wu and Wu, 1988). Some of these techniques may be successfully adapted for in vivo or ex vivo use.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa, and hepatoma cells. Nicolau et al., (1987) accomplished successful liposome-mediated gene transfer in rats after intravenous injection.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr: that confers resistance to methotrexate; gpt, that confers resistance to mycophenolic acid; neo, that confers resistance to the aminoglycoside G418; and hygro, that confers resistance to hygromycin.

Pharmaceutical Compositions

Where clinical applications are contemplated, it may be necessary to prepare pharmaceutical compositions—expression vectors, virus stocks, proteins, antibodies and drugs—in a form appropriate for the intended application. Generally, this will entail preparing compositions that are essentially free of impurities that could be harmful to humans or animals.

One generally will desire to employ appropriate salts and buffers to render delivery vectors stable and allow for uptake by target cells. Buffers also are employed when recombinant cells are introduced into a patient. Aqueous compositions of the present invention comprise an effective amount of the protein or peptide, dissolved or dispersed in a pharmaceutically acceptable carrier or aqueous medium. Such compositions also are referred to as innocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the proteins or peptides of the present invention, its use in therapeutic compositions is contemplated. Supplementary active ingredients also can be incorporated into the compositions.

The active compositions of the present invention may include classic pharmaceutical preparations. Administration of these compositions according to the present invention may be accomplished via any common route so long as the target tissue is available via that route. This includes oral, nasal, buccal, rectal, vaginal or topical. Alternatively, administration may be by orthotopic, intradermal, subcutaneous, intramuscular, intraperitoneal, intraarterial or intravenous injection. Such compositions normally would be administered as pharmaceutically acceptable compositions, described supra.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it is preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Therapeutic Agents

In certain embodiments, chemotherapeutic agents may be attached to a targeting peptide or fusion protein for selective delivery to a tumor. Agents or factors suitable for use may include any chemical compound that induces DNA damage when applied to a cell. Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), famesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. An alkylating agent, may include, but is not limited to, a nitrogen mustard, an ethylenimene, a methylmelamine, an alkyl sulfonate, a nitrosourea or a triazines. They include but are not limited to: busulfan, chlorambucil, cisplatin, cyclophosphamide (cytoxan), dacarbazine, ifosfamide, mechlorethamine (mustargen), and melphalan.

Antimetabolites

Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

Natural Products

Natural products generally refer to compounds originally isolated from a natural source, and identified has having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. They include such compounds as vinblastine (VLB) and vincristine.

Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Examples of antitumor antibiotics include, but are not limited to, bleomycin, dactinomycin, daunorubicin, doxorubicin (Adriamycin), plicamycin (mithramycin) and idarubicin.

Hormones

Corticosteroid hormones are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

Progestins such as hydroxyprogesterone caproate, medroxyprogesterone acetate, and megestrol acetate have been used in cancers of the endometrium and breast. Estrogens such as diethylstilbestrol and ethinyl estradiol have been used in cancers such as breast and prostate. Antiestrogens such as tamoxifen have been used in cancers such as breast. Androgens such as testosterone propionate and fluoxymesterone have also been used in treating breast cancer. Antiandrogens such as flutamide have been used in the treatment of prostate cancer. Gonadotropin-releasing hormone analogs such as leuprolide have been used in treating prostate cancer.

Miscellaneous Agents

Some chemotherapy agents do not fall into the previous categories based on their activities. They include, but are not limited to, platinum coordination complexes, anthracenedione, substituted urea, methyl hydrazine derivative, adrenalcortical suppressant, amsacrine, L-asparaginase, and tretinoin. It is contemplated that they may be used within the compositions and methods of the present invention.

Platinum coordination complexes include such compounds as carboplatin and cisplatin (cis-DDP).

An anthracenedione such as mitoxantrone has been used for treating acute granulocytic leukemia and breast cancer. A substituted urea such as hydroxyurea has been used in treating chronic granulocytic leukemia, polycythemia vera, essental thrombocytosis and malignant melanoma. A methyl hydrazine derivative such as procarbazine (N-methylhydrazine, MIH) has been used in the treatment of Hodgkin's disease. An adrenocortical suppressant such as mitotane has been used to treat adrenal cortex cancer, while aminoglutethimide has been used to treat Hodgkin's disease.

Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bc_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

Non-limiting examples of pro-apoptosis agents contemplated within the scope of the present invention include gramicidin, magainin, mellitin, defensin, cecropin, (KLAKLAK)₂ (SEQ ID NO: 1), (KLAKKLA)₂ (SEQ ID NO: 2), (KAAKKAA)₂ (SEQ ID NO: 3) or (KLGKKLG)₃ (SEQ ID NO: 4).

Angiogenic Inhibitors

In certain embodiments the present invention may concern administration of targeting peptides attached to anti-angiogenic agents, such as angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline.

Dosages

The skilled artisan is directed to “Remington's Pharmaceutical Sciences” 15th Edition, chapter 33, and in particular to pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA Office of Biologics standards.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Bone Marrow Targeting Peptides

A non-limiting example of an organ of interest for targeting petides is bone marrow. Bone is the preferred site of metastasis in the large majority of patients with prostate cancer (Fidler, 1999). This striking selectivity has been viewed as an example of site-specific interactions that were essential to cancer progression (Rak, 1995; Zetter, 1998). Despite the clinical relevance, little is known about the mechanisms that control prostate cancer spread to bone. In addition, there were no effective strategies for targeting therapeutic agents for the treatment of metastatic prostate cancer (Brodt et. al, 1996).

A subset of peptides capable of selective homing to bone marrow through the circulation is likely to simulate the behavior of prostate cancer cells during bone metastasis formation. The vascular markers targeted by using phage display might also be utilized by tumor cells to metastasize. This concept has already been proven to be true for lung-homing peptides. Peptides that home to lung blood vessels inhibit experimental metastasis. These results fit a “modified seed and soil” model, in which the basis for site-specific metastasis is the presence of homing receptors in blood vessels of certain tissues to which metastasis preferentially occurs. Such selective vascular markers are exposed to tumor cells during adhesion, the first step of the metastastic cascade. Isolation of bone marrow-homing peptides is of utility for identifying those vascular markers that mediate prostate cancer cell homing during the metastatic process, and for potential therapeutic intervention in preventing metastases to bone, or in selectively imaging and/or treating cancer that has already metastasized to bone.

Methods

In vitro screenings using human bone were used to characterize peptides that bind to human bone marrow surfaces. The compositions and methods disclosed herein were of use to develop new anti-prostate cancer therapeutic strategies that focus on the prevention and treatment of bone metastasis.

Screening of Phage Display Libraries on Human Bone Marrow:

Fresh human ribs removed during surgery for access to underlying tumors were sectioned to expose the bone marrow surface. No significant damage to the bone marrow was inflicted to the tissue and the morphology was well preserved during after the procedure. The bone samples were washed (gently) 5 times with ice cold DMEM/0.5% BSA (sterile filtered) containing protease inhibitors (PMSF, aprotinin, leupeptin), “DMEM/BSA-PI”. This buffer was used for the whole procedure.

Incubation with the phage library and all the other steps were performed in a humidified chamber using a small petri dish inside a large one. The large dish was covered with water soaked paper towels. The rib surfaces were incubated with DMEM/BSA-PI at 4° C. for 40 minutes. The samples were then lifted and transferred to a new small petri dish. On that new dish, a large drop of library diluted in DMEM/BSA-PI was added. Care was taken so that enough buffer solution covered the entire surface of the bone marrow when the sample was placed face down.

The phage library remained in contact with the bone marrow surface for 1-2 hours, at 4° C. The tissue was then washed gently about 5-10 times with 1 ml of DMEM/BSA-PI. K91 E. coli were infected and phage recovery was performed at room temperature for 1-2 hours. The bone sample was removed after infection and aliquots of the K91 culture were plated in serial dilutions. After the antibiotic concentrations were adjusted the culture was grown overnight. These were the bulk recovered phage. It was necessary to grow 200 individual colonies after each round for RII, and also sequence phage from every round. The inputs were titrated retrospectively after each round. Triplicate platings were done for statistical significance. For sequencing, it was important to obtain colonies that were well spread and distant from each other.

The cultures were processed for successive rounds of panning as follows: K91 E. coli cultures were spun at 8,000, and PEG precipitated. Phage formed a white pellet that was usually visible and has to be re-suspended in 100-200 μl of PBS. Usually half (50 μl) of the material could be used for the next round of panning. The process was repeated for at least three rounds. To evaluate if there was selectivity during the screenings, control phage containing no peptide insert (Fd tet) were tested side-by-side at similar concentrations. The number of phage recovered using bulk vs. single colony preps also showed if there were differences based on selectivity.

Results

Human Bone Marrow Targeting Ex-Vivo

Human ribs were obtained from the Department of Pathology at the MD Anderson Cancer Center. These materials were generated during kidney and lung cancer surgeries and removed to provide access to the tumor site. The ribs were sectioned in half to expose the marrow surface. No significant damage was inflicted on the tissue and morphology was preserved during these procedures. This material was suitable for the isolation of specific peptides that bind to human bone marrow, listed below. Bone-binding assays were performed in vitro to confirm that a number of these peptides bind to the rib surfaces. Targeting peptide sequences that homed to human bone marrow were shown in Table 3.

TABLE 3
Phage recovered from human bone marrow surfaces in multiple
rounds of selection
CGLRCPLVCPGGC (SEQ ID NO:5)
CPVGCGGGCRPAC (SEQ ID NO:6)
CEVLCGVDCSNRC (SEQ ID NO:7)
WVSPVLG (SEQ ID NO:8)
VLGPRAM (SEQ ID NO:9)
LVGKWPY (SEQ ID NO:10)
PSRRLGS (SEQ ID NO:11)
APNTPVL (SEQ ID NO:12)
CRLADKELC (SEQ ID NO:13)
CRLSLPELC (SEQ ID NO:14)
TSRFSL (SEQ ID NO:15)
SLHRVAR (SEQ ID NO:16)
PLLVRTV (SEQ ID NO:17)
GRGRMTS (SEQ ID NO:18)
Phage recovered from bone core biopsies appearing frequently in multiple
rounds of selection
CSSPFHDSC (SEQ ID NO:19)
CTSAASGLC (SEQ ID NO:20)
CPGPLNPPC (SEQ ID NO:21)
CAFNNDDVC (SEQ ID NO:22)
CQISIWRTC (SEQ ID NO:23)
CSPPLTRWC (SEQ ID NO:24)
CQSDCIDLC (SEQ ID NO:25)
CGAGCGVPCPGGC (SEQ ID NO:26)

Statistical Analysis of the Peptide Motifs

A system has been designed to analyze the data resulting from peptide library screenings, adapted from the SAS package. The system is available upon request from the M. D. Anderson Cancer Center. Table 4 summarizes the data compiled after 400 phage clones selected ex-vivo were entered in the program. A peptide library pool was used in these experiments. Shown are peptide motifs that appeared more than 3 times.

Table 4. Sequences of motifs in phage that bind to human bone marrow ex-vivo.

| Motifs | Frequency |
|---|---|
| FHD (SEQ ID NO:27) | 6 |
| FHDS (SEQ ID NO:28) | 6 |
| HDS (SEQ ID NO:29) | 6 |
| PFH (SEQ ID NO:30) | 6 |
| PFHD (SEQ ID NO:31) | 6 |
| PFHDS (SEQ ID NO:32) | 6 |
| SPF (SEQ ID NO:33) | 6 |
| SPFH (SEQ ID NO:34) | 6 |
| SPFHD (SEQ ID NO:35) | 6 |
| SPFHDS (SEQ ID NO:36) | 6 |
| SSP (SEQ ID NO:37) | 6 |
| SSPF (SEQ ID NO:38) | 6 |
| SSPFH (SEQ ID NO:39) | 6 |
| SSPFHD (SEQ ID NO:40) | 6 |
| SSPFHDS (SEQ ID NO:41) | 6 |
| SSF (SEQ ID NO:42) | 5 |
| SSS (SEQ ID NO:43) | 5 |
| FSV (SEQ ID NO:44) | 4 |
| FSVT (SEQ ID NO:45) | 4 |
| SAA (SEQ ID NO:46) | 4 |
| SFS (SEQ ID NO:47) | 4 |
| SFSV (SEQ ID NO:48) | 4 |
| SFSVT (SEQ ID NO:49) | 4 |
| SSFS (SEQ ID NO:50) | 4 |
| SSFSV (SEQ ID NO:51) | 4 |
| SSSF (SEQ ID NO:52) | 4 |
| SSSFS (SEQ ID NO:53) | 4 |
| SSSFSV (SEQ ID NO:54) | 4 |
| SSSFSVT (SEQ ID NO:55) | 4 |
| SSFSVT (SEQ ID NO:56) | 4 |
| SVT (SEQ ID NO:57) | 4 |

The skilled artisan will realize that the bone marrow targeting peptide sequences identified herein will be of use for numerous applications within the scope of the present invention, including but not limited to targeted delivery of therapeutic agents or gene therapy, in vivo imaging of normal or diseased organs, tissues or cell types, identification of receptors and receptor ligands in organs, tissues or cell types, and therapeutic treatment of a number of human diseases, particularly metastatic prostate cancer.

### Example 2

Fingerprinting the Circulating Pool of Immunoglobulins Elicited against Prostate Cancer in Human Patients provides a Novel Prostate Cancer marker that is Prognostic for Disease Progression

In another embodiment illustrated in the present non-limiting example, phage libraries are screened against the pool of immunoglobulins from an individual with a disease state or other characteristic. In this embodiment, the antibody pool provides a structural sampling of ligands targeted to naturally occuring receptors, some of which may constitute novel disease markers. Biopanning against an antibody pool may be used to identify disease markers and to further characterize the molecular events underlying the disease state.

The present example shows the feasibility of this approach by identifying a novel marker for prostate cancer. The results further show that this marker has prognostic value for predicting which individuals with prostate cancer are likely to have an unfavorable clinical outcome, resulting in death of the patient. As discussed above, there is a great need in the field of prostate cancer for a reliable method to separate those individuals whose prostate cancer will prove lethal (and therefore are candidates for more agressive therapeutic intervention) from individuals who will not die from prostate cancer. The present example represents a significant advance in prostate cancer prognosis and illustrates the utility of the claimed methods and compositions.

The skilled artisan will realize that although the present example deals with prostate cancer, the methods and compositions disclosed are suitable for use with any disease state or condition in which the host immune system is likely to produce antibodies against a molecular marker associated with the disease or condition.

In the present example, the repertoire of circulating antibodies from the serum of prostate cancer patients with advanced disease was used to screen a phage display library. Certain peptides binding to those antibodies correspond to tumor antigens expressed in bone metastasis of prostate cancer. A panel of prostate cancer serum samples from patients with recorded clinical outcome was screened by an ELISA assay against those peptides. The results show that reactivity against one particular peptide (“peptide C”) can be used to identify patients with metastatic androgen-independent prostate cancer. Moreover, patients with detectable levels of circulating antibodies against peptide C exhibited decreased survival compared to individuals without such antibodies.

Methods

Sera was selected from patients diagnosed with androgen-dependent and androgen-independent prostate cancer. A CX6C peptide library was screened against this pool of IgGs in a two-step procedure. First, the peptide library was pre-cleared against a pool of purified IgGs from normal serum samples using Protein G. This step removed peptides from the phage display library that bound to immunoglobulins from patients without prostate cancer. Next, the pre-cleared peptide library was screened against the pool of purified IgGs from the serum of prostate cancer patients. This step selected peptides binding specifically to IgGs elicited against prostate cancer.

Human Sera and Tissue Samples.

Human plasma samples were prospectively collected from 91 patients with locally advanced, metastatic androgen-dependent and metastatic androgen-independent adenocarcinoma of the prostate. The following criteria were applied to the locally advanced group: no evidence of regional and distant metastasis, clinical stage T1c or T2, with high grade disease (Gleason score 8-10) on initial biopsy or clinical stage T2b-T2c with Gleason score 7 and PSA >10 or clinical stage T3, negative bone scan and CT of the abdomen and pelvis. Patients in the metastatic androgen-dependent group were responsive to androgen ablation (either combined or LHRH agonist alone) at the time blood samples were collected. A castrate serum testosterone level <50 ng/dl and a rising PSA for two consecutive measurements at least two weeks apart defined androgen independence.

For biopanning, sera was examined from three metastatic androgen-independent and one metastatic androgen-dependent prostate cancer patients. Plasma from 34 healthy individual donors (eleven males) was obtained from the Blood Bank at the University of Texas M. D. Anderson Cancer Center (UTMDACC). Archived tissue paraffin blocks were obtained from the Department of Pathology at UTMDACC. The blood samples were initially allowed to clot at room temperature and then centrifuged to separate the cellular component from the supernatant. Aliquots of supernatant were promptly frozen and stored at −80° C. until assayed.

Biopanning.

A 6-mer cyclic peptide library (CX₆C), constructed as described above, was used for the biopanning. To select peptides specific to the serum antibodies of cancer patients, a pre-clearing stage was employed to remove non-specific peptides by pre-absorbing the peptide library onto purified IgGs from pooled normal serum (five healthy male individuals). The pre-cleared peptide library was screened onto the purified IgGs from the serum of prostate cancer patients. In brief, 10⁹transducing units (T.U.) of a CX₆C cyclic peptide library phage were incubated with IgG antibodies from 50 μl of normal serum immobilized on 50 μl of protein G (Gibco BRL) for 1 hour at 4° C. This was followed by affinity selection on the immobilized IgG antibodies from prostate cancer patient serum for 2 hours at 4° C. Phage peptides specifically bound to IgGs elicited against prostate cancer were eluted with 100 μl of 0.1 M glycine buffer, pH 2.2, neutralized by the addition of 10 μl 1M Tris pH 9.0, and used to infect E. coli strain K91. Ten-fold serial dilutions of the infected solution were spread onto agar plates containing 40 μg/ml of tetracycline and grown overnight. Two hundred colonies were picked, amplified, and precipitated for a subsequent round of panning. A total of three rounds were performed. Individual phage clones were picked for PCR and the insert DNA was sequenced.

Enzyme-Linked Immunosorbent Assay and Peptide Inhibition Study.

A 20 μg/ml solution of purified GST or GST-fusion proteins in 0.1M NaHCO₃ was used to coat maxisorp multi-well plates (Nalge Nunc International Corporation) and incubated overnight at 4° C. The plates were blocked in a blocking buffer (4% milk, 2% casein, and 0.05% Tween-20) for 3-4 hours. A series of 100-fold dilutions (1:100-1:1200) of sera from prostate cancer patients or healthy individuals was added and incubated for 1.5 hours and then washed five times with washing buffer (1% milk, 0.5% casein, and 0.025% Tween-20), followed by incubation at 4° C. with anti-human allaline phosphatase-conjugated antibodies (Gibco). The plates were then washed six times in washing buffer and developed using p-NPP (Sigma) as a substrate. An automatic ELISA plate reader (BIO-TEK instrument) recorded the results at OD405 nm.

Antibody Biotinylation.

GST-fusion proteins containing the peptide sequence from patient C were coated on multi-well plates. After incubating the plates with the patient's serum, the plates were washed. The bound IgG antibodies were eluted with 50 μl of 0.1 M glycine buffer, pH 2.2, neutralized by addition of 10 μl 1 M Tris pH 9.0, and dialyzed in PBS overnight followed by concentration of the antibody using Centricon-30 (Millipore) filters. The purified antibody (500 μg) was coupled to biotin according to the manufacturer's instructions (Vector). The biotinylated antibody was analyzed by SDS-gel electrophoresis.

Immunohistological Staining.

Paraffin sections from patient C were stained with purified biotinylated antibodies and peptide antibodies by immunoperoxidase detection using the Dako antigen retrieval kit and DAB as a substrate. All of the sections (4 μm) were counter-stained with hematoxylin. The biotinylated immunopurified antibodies were used at a dilution of 1:100. Peptide C antibodies and purified pre-immune antibodies were used at 0.01 μg/μl. For the inhibition staining, peptide C antibodies were pre-incubated for 30 minutes at room temperature with the GST-peptide C (500μg) prior to staining. Peptide antibodies were generated in rabbits and purified using a T-gel immunoglobulin purification kit and protein G column (Pierce).

Statistical Analysis.

Probabilities of survival for each group were estimated using the Kaplan-Meier method. A log-rank test was implemented in order to detect significant differences between the groups. Reactivity was considered to be detected if the ratio between GST-peptide and GST alone was greater or equal to two by the ELISA data. The Cox proportional hazards model was applied to analyze the effect of single and multiple risk factors in association with survival. Martingale residual plots were used to assess the proportional hazard assumption. P-values less than 0.05 were considered significant. All analyses were performed using SPLUS statistical software.

Results

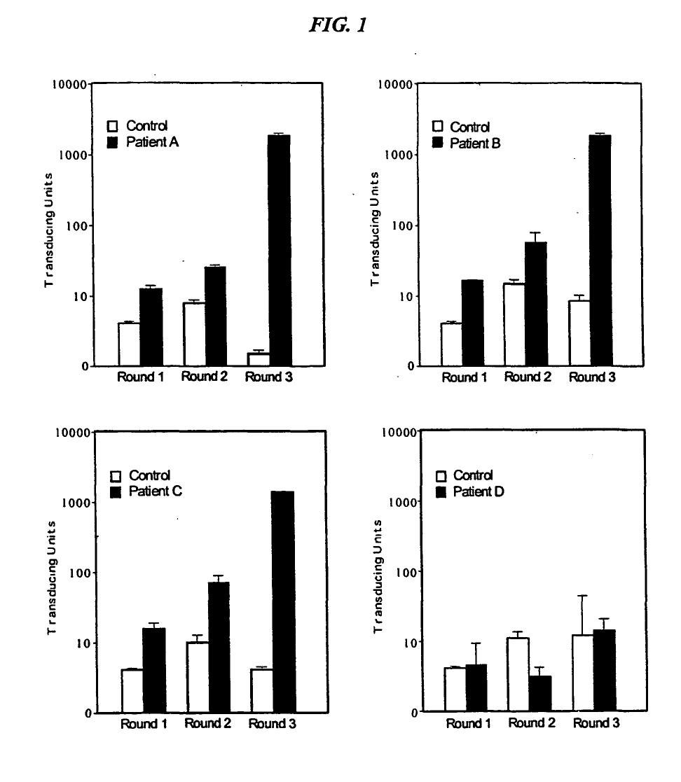

After three rounds of selection (FIG. 1), a striking enrichment (log scale) was observed in three out of the four serum samples examined. Individual phage clones from the second and third rounds of selection from serum samples A, B, and C were sequenced. The peptide motifs CHQKPWEC (SEQ ID NO: 58) from sample A and CKDRFERC (SEQ ID NO: 59) from sample B represented 100% of the clones analyzed, whereas the peptide motif CNVSDKSC (SEQ ID NO: 60) from sample C appeared in 55% of the clones analyzed.

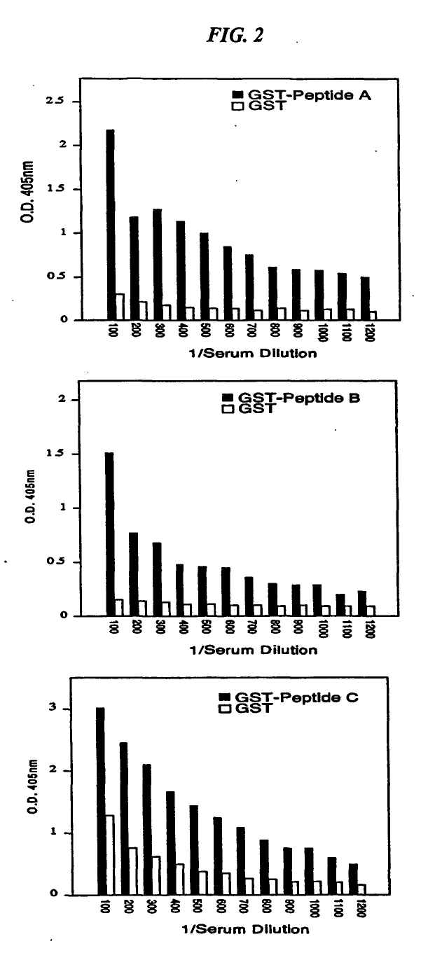

ELISA was performed to assess if the peptides could be specifically recognized by the antibodies present in the serum of the patients selected for the screenings. Peptides A (CHQKPWEC, SEQ ID NO: 58), B (CKDRFERC, SEQ ID NO: 59), and C (CNVSDKSC, SEQ ID NO: 60) were produced as GST-fusion proteins and immobilized onto microtiter wells, along with GST alone as a negative control. For each sample tested, a series of 100-fold dilutions was performed. Little reactivity occurred with the GST control, whereas strong reactivity occurred with the GST-fusion peptides (FIG. 2). The reactivity of each serum against peptides A, B and C was inhibited by the corresponding synthetic peptides (data not shown).

The reactivity profile of 91 sera obtained from clinically annotated prostate cancer patients was examined. Sera from 34 healthy individuals were also tested as a control for specificity. ELISA was performed to evaluate each serum using the three selected peptides (A, B, and C). The sera obtained from patients with prostate cancer were divided into three groups: 35 originated from patients with localized adenocarcinomas, 27 from patients with metastatic androgen-dependent disease, and 29 originated from patients with metastatic androgen-independent disease.

Among the normal male serum samples tested there was no significant reactivity with peptide C (0%), whereas some reactivity was observed with peptides A (73%) and B (9%) (not shown). Peptide C was strongly recognized by antibodies in the serum of patients in the metastatic androgen-independent group (76%) (not shown). The serum samples obtained from patients with locally advanced adenocarcinoma or with metastatic androgen-dependent disease showed a lesser degree of reactivity against peptide C (31% and 33%, respectively) (not shown) suggesting that positive reactivity with this peptide correlates with late stage metastatic prostate cancer.

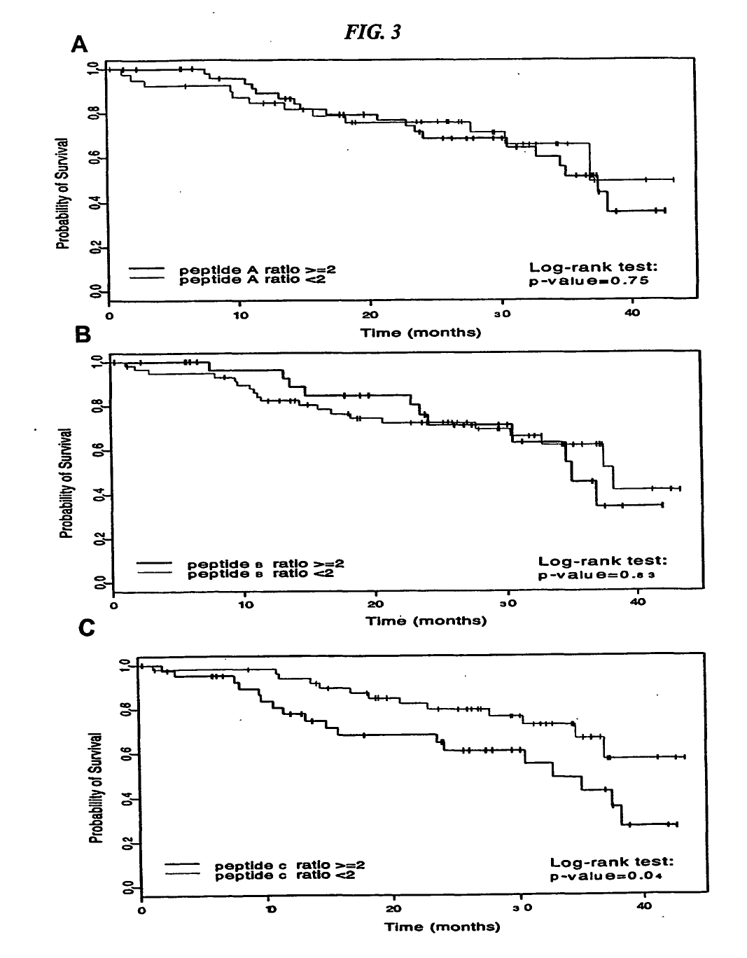

Univariate Cox modeling and Kaplan-Meier curve estimates were used to compare the survival outcome between the positive reactive and non-reactive groups for each of the three peptides (FIGS. 3A-3C). Positive reactivity to peptide C (n=42) was significantly associated with shorter survival outcome (log rank test P=0.04), while no statistically significant associations were detected for peptides A and B (FIGS. 3A-3C). The 3 year survival rate for the individuals showing reactivity against peptide C was 42%, compared to a 67% survival rate in the non-reactive group. Thus, reactivity against peptide C was associated with an almost 40% decrease in the number of patients surviving after 3 years.

A multivariate Cox proportional hazards model was fitted to include PSA (prostate-specific antigen) as an additional covariate (Table 5). Both elevated PSA levels and positive reactivity to peptide C were simultaneously associated with shorter survival risk (P=0.03 and P=0.006 respectively), suggesting that peptide C reactivity is an independent prognostic marker. The relative risk estimates for peptide C reactivity corresponding to the univariate and multivariate models did not differ significantly, suggesting that there was no confounding effect of serum PSA. Taken together, the data suggest a strong correlation between peptide C reactivity and the most advanced stage of prostate cancer (metastatic androgen-independent disease).

Antibodies against peptide C were examined by immunohistochemistry to determine whether they would recognize tumor-associated antigens, using tissue sections from bone marrow metastasis (derived from surgical specimens from patient C). Strong tumor staining was observed using immunopurified antibodies from patient C's serum (not shown). Specific immunostaining was also observed using a rabbit polyclonal antibody against the synthetic form of peptide C (not shown). However, no staining was observed with pre-immune antibodies (not shown). The staining was completely inhibited by the GST-fusion protein containing peptide C (not shown). Normal prostate tissue was completely negative when tested for reactivity with the anti-peptide C polyclonal antibody (not shown).

These results demonstrate that antibodies from a metastatic prostate cancer patient could be used to screen a phage display library for a prostate cancer marker —peptide C. The presence of circulating antibodies against peptide C in the serum of prostate cancer patients was indicative of a substantial decrease in patient survival after 3 years. Purified antibodies against peptide C bound to an endogenous antigen in metastatic bone marrow samples, but not to normal bone marrow tissue. These results show the utility of the antibody screening method and provide a novel and significant new prostate cancer marker of great value for prostate cancer prognosis. The skilled artisan will realize that the disclosed peptide sequences are of value for a variety of applications, including but not limited to prostate cancer detection, diagnosis and prognosis, therapeutic vaccine development, rapid immunodiagnostic screening, and the identification of the natural antigen. In many cases where tumor antigen are often unknown, disease-specific antigens identified using this approach could be employed to define common or unique features in the immune response of individuals to the same disease, i.e., immunofingerprinting the immune response against a given antigen.

TABLE 5
Univariate and multivariate analysis of survival in patients
with prostate cancer by the Cox's prop rtional-hazards model

| variable | coefficient | relative risk | Confidence interval | p-value |
|---|---|---|---|---|
| Univariate | | | | |
| Log PSA | 0.254 | 1.29 | 1.05-1.58 | 0.016 |
| Peptide A ratio | 0.217 | 1.24 | 0.82-1.89 | 0.31 |
| Peptide B ratio | 0.152 | 1.16 | 0.86-1.57 | 0.32 |
| Peptide C ratio | 0.531 | 1.7 | 1.22-2.37 | 0.002 |
| Multivariate | | | | |
| Log PSA | 0.213 | 1.24 | 1.02-1.50 | 0.03 |
| Peptide C ratio | 0.5 | 1.65 | 1.16-2.35 | 0.006 |

Additional peptide sequences reactive with antibodies from patient C were identified (Table 6). Each of these peptide sequences was compared to the Swiss Protein database of known proteins to search for amino acid identity. No such homologies were identified for targeting peptides of SEQ ID NO: 60 to SEQ ID NO: 65.

The aligned sequences were also used to search unique epitopes by cross- referencing the peptide sequences in Table 6 using the MacVector software package (Oxford Molecular Group). To confirm these findings, a short peptide database and analysis software were developed in collaboration with the Department of Biostatistics at UTMDACC. This biostatistical program determines the cumulative frequency by which any combination of 3 or more amino acid residues occurs. It was found that the conserved regions identified from the LALIGN program matched frequently occurring residue sequences identified from the biostatistical analysis.

Table 6 presents a few of the selected targeting peptides, including peptide C. Peptide motifs with the consensus sequences CNXSDKSC (SEQ ID NO: 61) or CNXTDKSC (SEQ ID NO: 62) were identified.

TABLE 6
Peptides isolated by biopanning on immunoglobulins from a
prostate cancer patient.

| Peptide Sequence | Frequency |
|---|---|
| CNX^{S}/_{T}DKSC | 96% |
| (SEQ ID NO:61 and | |
| SEQ ID NO:62) | |
| CNVSDKSC | 55% |
| (SEQ ID NO:60) | |
| CNWTDKTC | 19% |
| (SEQ ID NO:63) | |
| CNITQKSC | 15% |
| (SEQ ID NO:64) | |
| CNKTDKGC | 7% |
| (SEQ ID NO:65) | |
| CKDRFERC | 4% |
| (SEQ ID NO:59) | |

### Example 3

Identification of the Endogenous Peptide C Antigen

The present methods are of use for identifying the naturally occurring analogs of targeting peptides. As described above, peptide C is apparently a mimeotope of an endogenous protein against which circulating antibodies are present in a high percentage of individuals with metastatic prostate cancer. The following methods were used to identify the endogenous peptide C antigen. As a metastatic prostate cancer marker, the endogenous antigen is important as a diagnostic and prognostic marker for prostate cancer, a potential therapeutic target for treatment of prostate cancer and a potential antigen for vaccine development against prostate cancer. The endogenous antigen may also be of significance in understanding the biochemical mechanisms underlying prostate cancer metastasis.

Methods and Results

The DU145 prostate cell line was used as the source material for purification of the peptide C antigen. Ten plates of cells were grown to confluence before harvesting. The cells were rinsed 3× in PBS before adding 750 ul of TM buffer (0.01M tris-CL, 0.002M MgCl2, 1% tritonX100) per plate. Cells from all plates were combined in a 50 ml tube by scraping the cells off the plates. The cells were sheared by passing 3× through a 22-gauge needle to separate the nuclei from rest of the cell. Aliquouts of 10 ul were visualized under a microscope to check for complete separation.

The homogenate was centrifuged at 800 rpm for 10 min and the supernatant (cytosolic/membrane fraction) transferred to new tubes (500 ul/tube). The remaining pellet containing the cell nuclear fraction was resuspended with 800 ul of nuclear lysis buffer (0.1% SDS, 0.5% Triton X100, 50 mM Tris-Cl, 10 mM NaCl).

Antibodies against peptide C were prepared by standard techniques. Briefly, synthetic peptide C was conjugated to KLH by ANASPEC and purified by HPLC. The conjugated peptide (100 μg) was injected into rabbits, who were subsequently boosted with the same peptide. Pre-immune serum was collected prior to the initial injection.

A 20 ul alilquot of supernatant fraction was run on a 4-20% SDS gel and Western blotting was performed using polyclonal rabbit anti-peptide C antibody. The antibody dilution used was 1:100 from the 9^{th} bleed of the rabbit. The ECL system (Amersham) was used to detect a band reacting with anti-peptide C antibody. A single band of about 80 kDa was detected from DU145 homogenate supernatant (not shown).

The 80 kDa band was excised for protein sequencing and further analysis. The gel slices containing excised protein were crushed in 3 ml of running buffer. The supernatant was recovered and concentrated using a Centricon-30 filter. About 40 ul of partially purified protein (excised from SDS-PAGE) was loaded onto an 8% SDS gel and Western blot analysis was performed. Pre-immune serum was used a control. The partially purified protein showed a somewhat diffuse band of about 80 kDa. Additional gel slices were removed from the SDS gel and analyzed for amino acid sequence by mass spectrometry.

Five peptide sequences were obtained from the protein excised from SDS gels. All five peptides matched portions of the 78 kDa glucose regulated protein (Table 7, GRP78, SEQ ID NO: 66, GenBank Accession Numbers CAB71335 and XM 044202). The locations of the five sequenced peptides within GRP78 are indicated in Table 7 in bold font. A commercial antibody against GRP78 reacted on Western blot with the purified peptide C antigen from DU145 cells showed positive reactivity (not shown). The original peptide C sequence (SEQ ID NO: 60) is not found within the GRP78 sequence (SEQ ID NO: 66), indicating that the epitope recognized in vivo by anti-peptide C antibodies is formed from discontiguous regions of the GRP78 protein.

TABLE 7
Sequence of Human GRP78

(SEQ ID NO:66)

MKLSLVAAMLLLLSAARAEEEDKKEDVGTVVGIDLGTTYSCVGVFK
NGRVEIIANDQGNRITPSYVAVFTPEGERLIGDAAKNQLTSNPENTV
FDAKRLIGRTWNDPSVQQDIKFLPFKVVEKKTKPYIQVDIGGGQTKT
FAPEEISAMVLTKMKETALEAYLGKKVTHAVVTVPAYFNDAQRQAT
KDAGTIAGLNVMRIINEPTAAAIAYGLDKREGEKNILVFDLGGGTF
DVSLLTIDNGVFEVVATNGDTHLGGEDFDQRVMEHFIKLYKKKTGK
DVRKDNRAVQKLRREVEKAKRALSSQHQARIEIESFYEGEDFSETLT
RAKFEELNMDLFRSTMKPVQKVLEDSDLKKSDIDEIVLVGGSTRIPK
IQQLVKEFFNGKEPSRGINPDEAVAYGAAVQAGVLSGDQDTGDLVL
LDVCPLTLGIETVGGVMTKLIPRNTVVPTKKSQIFSTASDNQPTVTIK
VYEGERPLTKDNHLLGTFDLTGIPPAPRGVPQIEVTFEIDVNGILRVTA
EDKGTGNKNKITTTNDQNRLTPEEEIERMVNDAEKFAEEDKKLKERID
TRNELESYAYSLKNQIGDKEKLGGKLSSEDKETMEKAVEEKIEWLES
HQDADIEDFKAKKKELEEIVQPIISKLYGSAGPPPTGEEDTAEKDEL

Based on these results, it appears that GRP78 is the endogenous antigen against which circulating antibodies are present in a high percentage of metastatic prostate cancer patients. These results are consistent with the reported characteristics of GRP78.

The GRP78 protein is a chaperone that is normally present in endoplasmic reticulum (Triantafilou et al., 2001). However, it has recently been reported to also exist as a cell surface protein, where it associates with MHC class I (Triantafilou et al., 2001). GRP78 is about 60% homologous to the hsp70 heat shock protein (U.S. Pat. No. 5,188,964). GRP78 is normally overexpressed in response to glucose starvation (U.S. Pat. No. 5,188,964). However, it is also induced in chicken embryo fibroblast cells transformed with Rous sarcoma virus (Stoeckle et al., 1988) and in breast cancer cells treated with estradiol (Kiang et al., 1997). Induction of glucose regulated proteins has been reported to be associated with increased resistance to adriamycin and etoposide in Chinese hamster ovary cells (Hughes et al., 1989). Conversely, induction of GRP78 apparently resulted in increased sensitivity to cisplatin in some human epidermoid carcinoma cells (Mese et al., 2001).

Induction of GRP78 in response to thapsigargin, a pro-apoptotic agent, has been reported in human rabdomiosarcoma cells and rat brain tumor cells (Delpino et al., 1998; Chen et al., 2000). Furuya et al. (1994) reported that glucose regulated protein was induced in rat and human prostate cancer cells treated with thapsigargin. Androgen independent prostatic cancer cells treated with thapsigargin underwent apoptosis within several days (Furuya et al., 1994). Circulating antibodies against GRP78 have been reported in ovarian cancer patients (Chinni et al., 1997).

The present disclosure is the first to report that antibodies reactive with GRP78 are present in a high percentage of individuals with metastatic prostate cancer, and that the presence of such antibodies in sera of prostate cancer patients is significantly associated with a substantial decrease in patient survival.

### Example 4

Phage Display Screening against Circulating Antibodies in Prostate Cancer Shows Antibody Progression Corresponding to Disease Progression

Examples 2 and 3 above illustrated the use of phage display screening against circulating antibodies from prostate cancer patients to identify a novel prostate cancer marker that is prognostic for patient survival. The present example illustrates a further embodiment of the methods, using phage display library screening to examine the progression in circulating antibodies accompanying disease progression.

The methods used were similar to those described in Example 2. A subtraction protocol was used, in which IgG from a normal individual was coupled to protein G chromatography beads. A cyclic CX₆C phage display library, prepared as described above, was exposed to the normal IgG's. Phage that did not bind to the normal IgG pool were collected and used for the next step. Antibodies from patient M (prostate cancer patient) were attached to fresh protein G chromatograpy beads. The phage display library that had been pre-exposed to normal IgG's was exposed to the IgG pool from patient M. After thorough washing of the column, the phage that bound to the prostate cancer IgG (but did not bind to normal IgG) was eluted and amplified. This procedure was followed for three rounds of screening and targeting peptides against patient M's antibodies were obtained.

Serum samples from the same patient were obtained from archival specimens and used to obtain targeting peptides. Patient M's serum from 1994 (early stage cancer), 1998 (intermediate stage) and 2000 (late stage) were used to obtain antibody targeting peptides as described above. These peptides were shown in Table 8. The numbers in parentheses indicate the number of phage exhibiting the sequence out of the total number of clones obtained.

TABLE 8
Peptides identified after three rounds of panning
on purified immunoglobulins from the serum of
prostate cancer patient M.

| 1994 Serum | 1998 Serum | 2000 Serum |
|---|---|---|
| CTFAGSSC (6/22) | CTFAGSSC (12/20) | CTFAGSSC (26/29) |
| (SEQ ID NO:67) | (SEQ ID NO:67) | (SEQ ID NO:67) |
| CNSAFAGC (1/22) | CSKKFVTC (3/20) | CNSAFAGC (1/29) |
| (SEQ ID NO:68) | (SEQ ID NO:83) | (SEQ ID NO:68) |
| CSYTFAGC (1/22) | CNSAFAGC (1/20) | CFPKRVTC (1/29) |
| (SEQ ID NO:69) | (SEQ ID NO:68) | (SEQ ID NO:87) |
| CSTFAGSC (1/22) | CKNKHTTC (1/20) | CPRSAKNC (1/29) |
| (SEQ ID NO:70) | (SEQ ID NO:84) | (SEQ ID NO:88) |
| CRDGYHHC (1/22) | CFETFAGC (1/20) | |
| (SEQ ID NO:71) | (SEQ ID NO:85) | |
| CSASDLSC (2/22) | CNNMYAGC (1/20) | |
| (SEQ ID NO:72) | (SEQ ID NO:86) | |
| CQNQYPEC (1/22) | CQNQYPEC (1/20) | |
| (SEQ ID NO:73) | (SEQ ID NO:73) | |
| CRASAMVC (1/22) | | |
| (SEQ ID NO:74) | | |
| CIDMTHQC (1/22) | | |
| (SEQ ID NO:75) | | |
| CISSPSNC (1/22) | | |
| (SEQ ID NO:76) | | |
| CNQSMWSC (1/22) | | |
| (SEQ ID NO:77) | | |
| CQFENGTC (1/22) | | |
| (SEQ ID NO:78) | | |
| CAVKSVTC (1/22) | | |
| (SEQ ID NO:79) | | |
| CNGFMGYC (1/22) | | |
| (SEQ ID NO:80) | | |
| CLTSENAC (1/22) | | |
| (SEQ ID NO:81) | | |
| CRASAMVC (1/22) | | |
| (SEQ ID NO:82) | | |

It is apparent that one sequence, CTFAGSSC (SEQ ID NO: 67) was the predominant antibody-binding peptide in all three samples. Further, the frequency of this targeting peptide as a fraction of the total pool of targeting peptides increased with time, suggesting that the antibody that bound this peptide also became more prevalent with tumor progression. It is also apparent that the diversity of targeting peptides binding to circulating antibodies decreased with disease progression, indicating that there was a corresponding decrease in antibody diversity.

It is not unusual for tumor cells to shed antigens into the circulation. Leukocytes may also be exposed to tumor antigens in situ. It is therefore expected that cancer patients in general will exhibit cirfculating antibodies against tumor antigens. Phage display libraries may be screened against cancer patient samples to identify targeting peptides that bind to antibodies against tumor specific or tumor associated antigens. The identified targeting peptides may be used, for example, to purify anti-tumor antibodies using affinity chromatograpy or other well-known techniques. The purified anti-tumor antibodies can be used in diagnostic kits to identify individuals with cancer. Alternatively, they could be attached to various therapeutic moieties, such as chemotherapeutic agents, radioisotopes, anti-angiogenic agents or pro-apoptosis agents and used for cancer therapy. The targeting peptides against anti-tumor antibodies may also be used to identify novel tumor specific or tumor-associated antigens, of diagnostic or therapeutic use. Phage display antibody libraries may also be constructed and screened against tumor targeting peptides. By this method, it is possible to isolate and purify large quantities of antibodies specific for tumor antigens.

The skilled artisan will realize that the CTFAGSSC (SEQ ID NO: 67) peptide could be used for ELISA or other immunoassays to screen the blood of individuals at risk for prostate cancer. The presence of an antibody that bound to SEQ ID NO: 67 in the serum of a patient would be indicative of prostate cancer. The peptide may also be used to prepare monoclonal or polyclonal antibodies that are of use for tumor diagnosis, imaging or therapy.

### Example 5

Identification of Receptor/Ligand Pairs: Endostatin receptors revealed by Phage Display

Endostatin is a recently characterized cell protein with reported anti-angiogenic properties (U.S. Pat. No. 6,174,861). It apparently acts at least in part by inhibiting endothelial cell proliferation, thus blocking the growth of new blood vessels (U.S. Pat. No. 6,174,861). Administration of endostatin is reported to inhibit tumor growth in model systems (U.S. Pat. No. 6,174,861). Despite its clinical significance, the mechanisms by which endostatin exerts these effects remain unknown. Elucidating the function of endostatin would be facilitated by identification of targeting peptides that bind to endostatin and potentially act as mimeotopes of endogenous endostatin ligands. Such peptides may also be of potential use as novel anti-angiogenic or anti-tumor agents.

To isolate endostatin-binding peptides, CX6C and CX7C phage libraries were screened using recombinant His-tag fusion proteins that contained endostatin coated onto microtiter wells. An immobilized His-tag control protein was used as a negative control for enrichment during the panning. Phage were sequenced from randomly selected clones after three rounds of panning as described (Koivunen et al., 1994, 1995; Pasqualini et al., 1995). Successful isolation of distinct sequences that interacted specifically with endostatin is reported in Table 9.

Randomly selected clones from round II and III were sequenced. Amino acid sequences of the phagemid encoded peptides were deduced from nucleotide sequences. The most frequent motifs found after panning using the indicated libraries were shown. Following the third round of selection, sequencing of the inserts from the individual phage selected on endostatin revealed a number of peptide motifs.

TABLE 9
Sequences displayed by phage binding to-endostatin

| | |
|---|---|
| CAGYAVDC | (SEQ ID NO:89) |
| CAMGSPEC | (SEQ ID NO:90) |
| CEAGRGGC | (SEQ ID NO:91) |
| CKLSGTRC | (SEQ ID NO:92) |
| CNGIVQVC | (SEQ ID NO:93) |
| CASSHAVC | (SEQ ID NO:94) |
| CWQGSVSC | (SEQ ID NO:95) |
| CMVGYIVC | (SEQ ID NO:96) |
| CWNRGSTC | (SEQ ID NO:97) |
| CPERGTRC | (SEQ ID NO:98) |
| CVNKYIPC | (SEQ ID NO:99) |
| CGTAEGVC | (SEQ ID NO:100) |
| CASPNLAC | (SEQ ID NO:101) |
| CDNGNASC | (SEQ ID NO:102) |
| CSQLKLGC | (SEQ ID NO:103) |
| CMGTKSSC | (SEQ ID NO:104) |
| CIDTSELC | (SEQ ID NO:105) |
| CGRVPQMC | (SEQ ID NO:106) |
| CAGFSSPC | (SEQ ID NO:107) |
| CSRSSFLC | (SEQ ID NO:108) |
| CIRPNDHC | (SEQ ID NO:109) |
| CVSRPRAC | (SEQ ID NO:110) |
| CMGQGQAC | (SEQ ID NO:111) |
| CADMQGTC | (SEQ ID NO:112) |
| CAIHDSEC | (SEQ ID NO:113) |
| CLVGAVQC | (SEQ ID NO:114) |
| CAYTNSLRC | (SEQ ID NO:115) |
| CFHMPPNTC | (SEQ ID NO:116) |
| CLTLKSNVC | (SEQ ID NO:117) |
| CLSKLHYVC | (SEQ ID NO:118) |
| CMESSSGLC | (SEQ ID NO:119) |
| CNRMTSYSC | (SEQ ID NO:120) |
| CPKDSSKMC | (SEQ ID NO:121) |
| CKAFQRHHC | (SEQ ID NO:122) |
| CCRLQVSHC | (SEQ ID NO:123) |
| CYSDRNMDC | (SEQ ID NO:124) |
| CYKPVHSPC | (SEQ ID NO:125) |
| CCETTTKDC | (SEQ ID NO:126) |
| CLKYEQRPC | (SEQ ID NO:127) |
| CPYDALASC | (SEQ ID NO:128) |
| CRMMALHEC | (SEQ ID NO:129) |
| CIVGAPRLC | (SEQ ID NO:130) |
| CRSSGFGTC | (SEQ ID NO:131) |
| CRHQSASAC | (SEQ ID NO:132) |
| CDGVLMFGC | (SEQ ID NO:133) |
| CYAAGNVFC | (SEQ ID NO:134) |
| CASSHAVC | (SEQ ID NO:135) |
| CNFAGPASC | (SEQ ID NO:136) |
| CRSLPPVRC | (SEQ ID NO:137) |
| CNPHKAQSC | (SEQ ID NO:138) |
| CRRDTYGRC | (SEQ ID NO:139) |
| CALPGGQIC | (SEQ ID NO:140) |

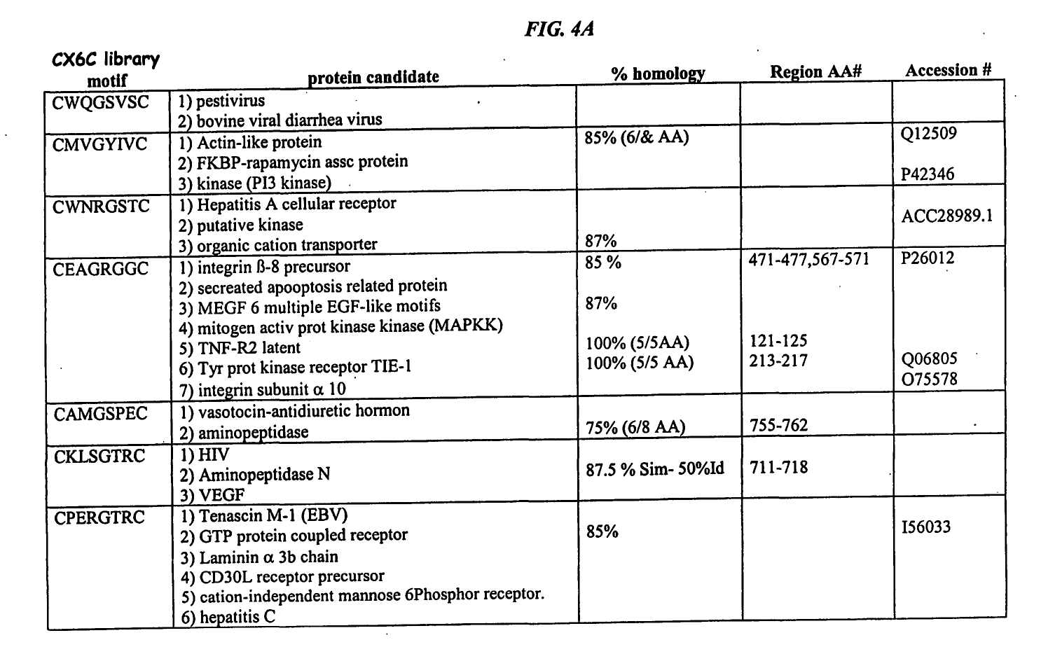

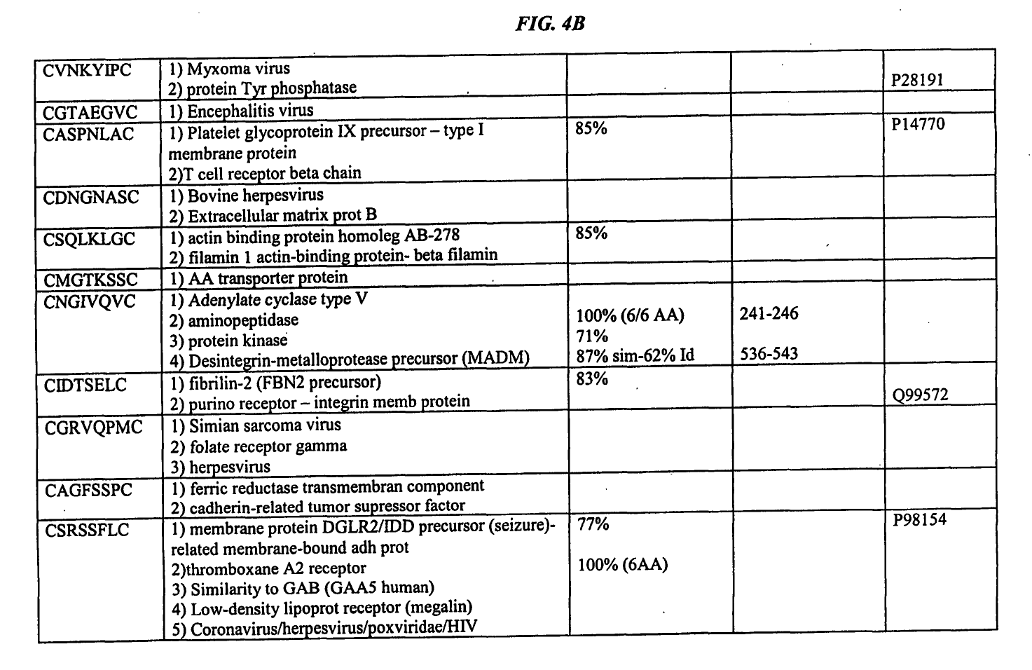

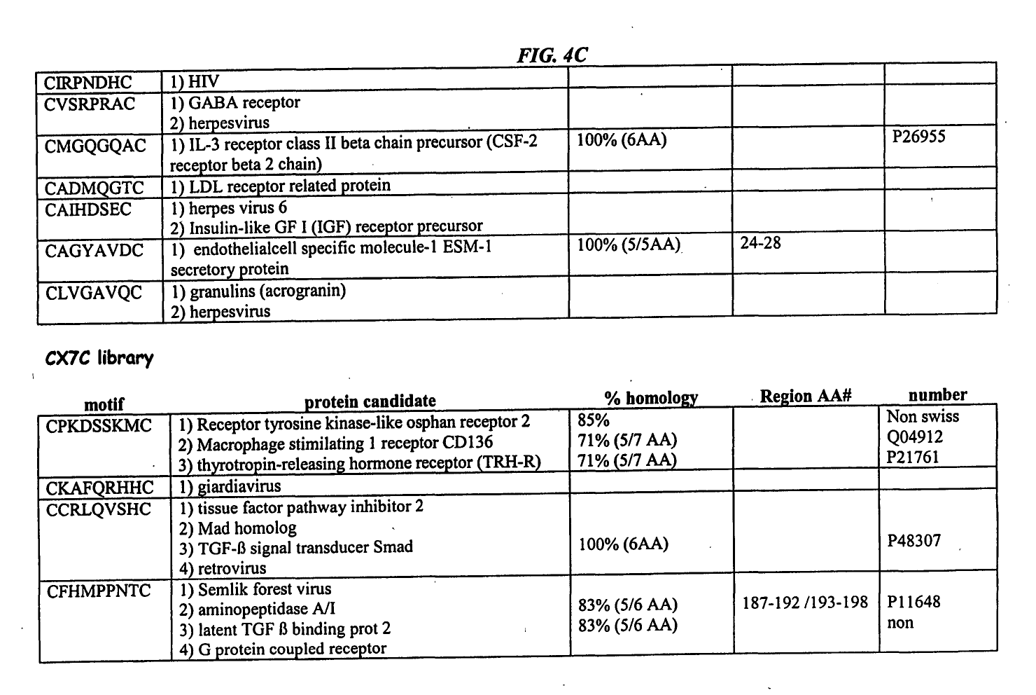

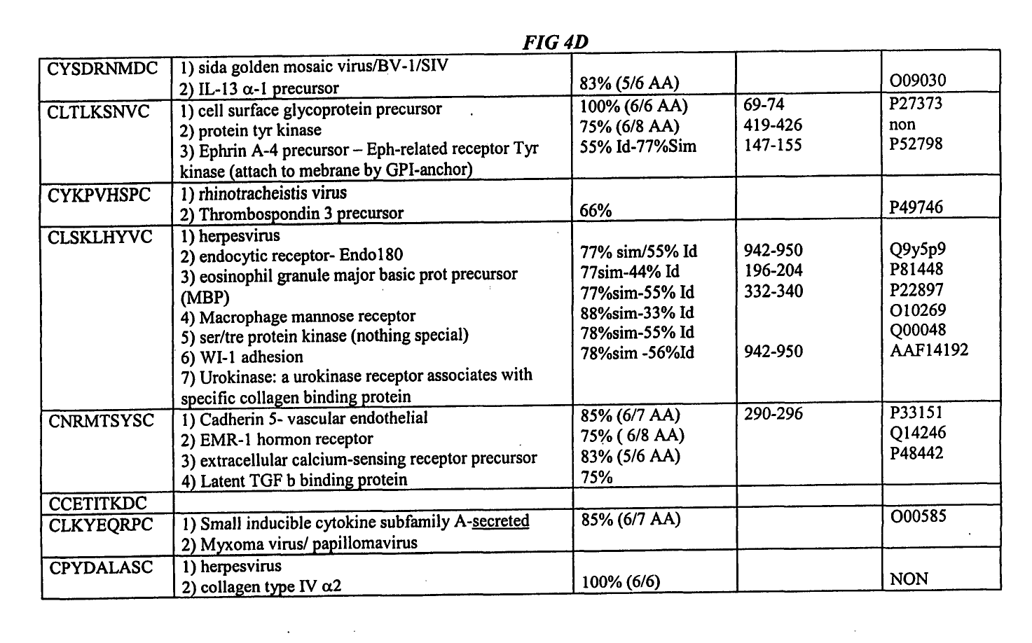

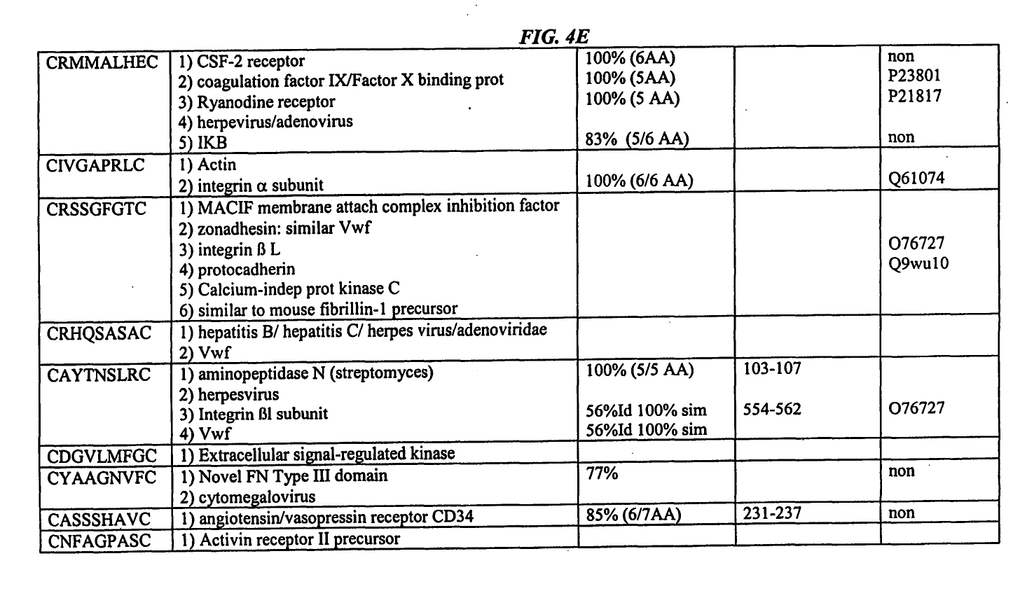

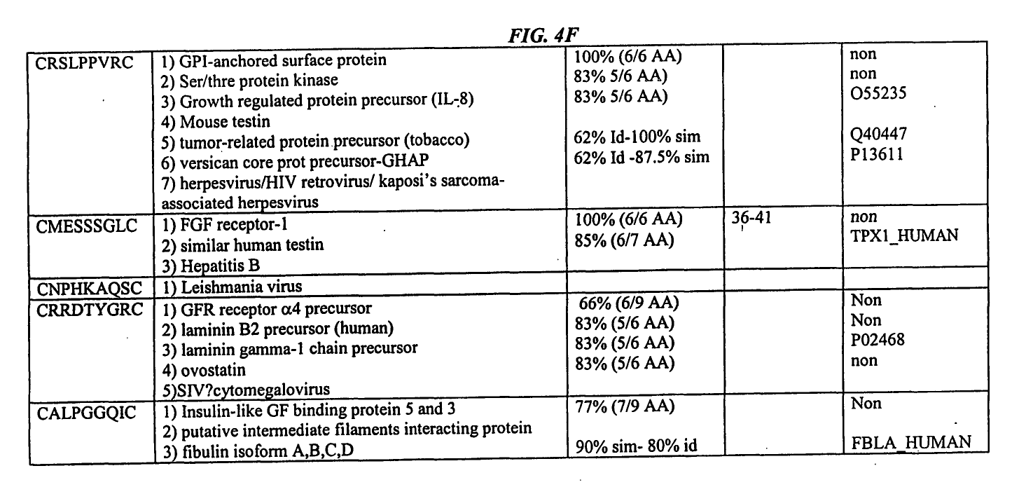

A homology search provided numerous examples of protein candidates for the endogenous endostatin receptors. These are shown in FIGS. 4A-4F. The skilled artisan will realize that the targeting peptide sequences identified herein are of potential use for the development of novel agents that may be either anti-angiogenic or pro-angiogenic, depending upon their interactions with endostatin, the endogenous endostatin receptor(s) and the binding interaction between endostatin and its receptor(s). Further, the putative endostatin receptor proteins identified by homology to the endostatin targeting peptides are potential targets for therapeutic treatment directed towards anti-angiogenesis.

### Example 6

Identification of Receptor/Ligand Pairs: Angiostatin Receptors Revealed by Phage Display

Another anti-angiogenic protein with reported anti-tumor activity is angiostatin, a proteolytic fragment of collagen XVIII. As with endostatin, the molecular mechanisms by which angiostatin induces these effects is unknown. The present example identifies angiostatin targeting peptides by phage display. Potential receptors for angiostatin are determined by homology with angiostatin targeting peptide sequences.

Materials and Methods

Antibodies, Proteins and Peptides

Anti-human angiostatin and anti-mouse endostatin (R&D systems: AF226, AF570), CD13 (Sigma St Louis, Mo.: L-9776), thrombospondin and hepatocyte growth factor (Calbiochem, San Diego, Calif.: 605225, 375228) were purchased from commercial sources. rh-Angiostatin and rh-Endostatin were produced by EntreMed, Inc. (Rockville, Md.). APN/CD13 enzyme and L-Alanine-p-nitroanilide hydrochloride substrate were purchased from Sigma, St Louis, Mo. (#L-9776; #A9325).

Selection of Angiostatin Targeting Phage and Phage Attachment Assay

A phage library displaying random cyclic peptides with the structure CX₇C (C, cysteine; X, any residue) was prepared. An aliquot of the library containing 3×10¹⁰ transducing units (TU) was screened with rhAngiostatin protein coated on microtiter wells as described (Koivunen et al 1993). In the first and second panning the amount of protein used was 10 μg/well. To increase the stringency of panning, the wells were coated with a decreased concentration of rhAngiostatin protein (100 ng/well) in the third panning. Phage remained bound after extensive washing. Bound phage were recovered by infection into F-pilus positive K91 bacteria. To determine the specificity of rhAngiostatin binding, selected phage were assayed as described but using rh-Endostatin and BSA proteins to coat the wells. In all assays, insertless phage (fdtet) was used as a control.

Protein-Protein Interaction

To assess protein-protein interactions, 100 ng/well of protein was coated in triplicate in 96-well plates. The plates were dried at 37° C., blocked with PBS and 3% BSA, and incubated with 10 μg/ml of rhAngiostatin in PBS+1% BSA for three hours at RT. The wells were washed three times with PBS, 3% BSA, 0.01% Tween 20 before incubation with anti-rhAngiostatin antibody (R&D Systems, Inc #AF226) at 1 μg/ml for 1 hour at RT. The wells were then washed three times with PBS, 3% BSA, 0.01% Tween 20 and incubated with HRP anti-goat antibody. After washing 3 times with PBS, 0.01% Tween 20 and once with PBS, the wells were developed with p-nitrophenyl phosphate. Binding of alkaline phosphate conjugate was detected colorimetrically at 450 nm. using an ELISA microplate reader.

Results

Panning Against Angiostatin with a CX7C Library.

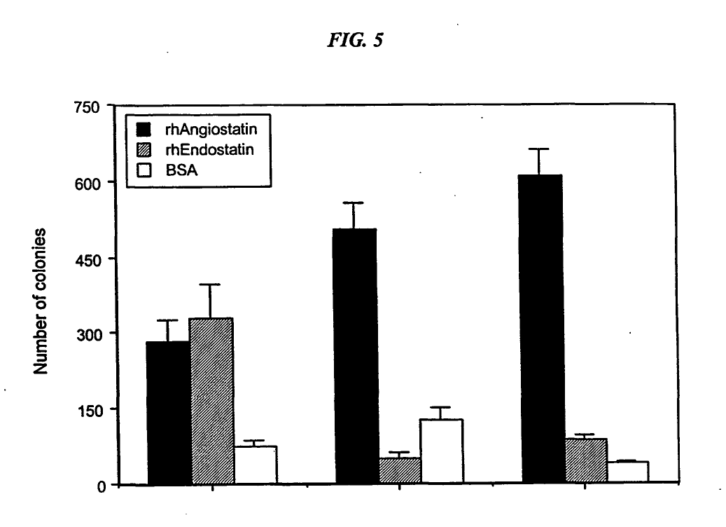

Enrichment of angiostatin binding phage was observed after only a single round of selection (FIG. 5). The degree of enrichment and selectivity of the recovered phage increased with each round of selection. After a single selection, the phage showed almost equal binding to angiostatin and endostatin (FIG. 5). After three rounds of selection, the recovered phage were highly selective for angiostatin versus endostatin and BSA (FIG. 5).

Phage were sequenced from randomly selected clones after three rounds of panning (Koivunen et al., 1994, 1995; Pasqualini et al., 1995). A number of distinct angiostatin-targeting sequences were identified (Table 10). Randomly selected clones from rounds II and III were sequenced. Amino acid sequences of the phagemid encoded peptides were deduced from nucleotide sequences. A consensus sequence appeared on the third round in 61% of the sequences examined (26 out of 42)—CWSLEXXKC (SEQ D NO: 141). Including the closely related sequence CWSAEWTKC (SEQ ID NO: 142) the consensus motif accounted for 71% of Angiostatin binding peptides. The ratios in the last two columns of Table 10 were calculated by dividing the number of colonies recovered from rhAngiostatin-coated wells by those recovered from rh-Endostatin or BSA wells.

TABLE 10
Sequences displayed by phage binding rh
Angiostatin.

| | | | rh- | |
|---|---|---|---|---|
| | | fre- | Angio/ | rh-Angio/ |
| Sequence | | quency | rhEndo | BSA |
| CWSLELSKC | (SEQ ID NO:143) | 10/42 | 630 | 630 |
| CWSLEFTKC | (SEQ ID NO:144) | 5/42 | 958 | 958 |
| CWSLEVAKC | (SEQ ID NO:145) | 5/42 | 486 | 1118 |
| CWSAEWTKC | (SEQ ID NO:142) | 4/42 | 124 | 892 |
| CWSLESLKC | (SEQ ID NO:146) | 4/42 | NT | NT |
| CWSLELLKC | (SEQ ID NO:147) | 2/42 | 489 | 734 |
| CGGREFWLC | (SEQ ID NO:148) | 1/42 | NT | NT |
| CGVVSRVGC | (SEQ ID NO:148) | 1/42 | NT | NT |
| CLAWVGGRC | (SEQ ID NO:149) | 1/42 | NT | NT |
| CQGKFSQRC | (SEQ ID NO:150) | 1/42 | NT | NT |
| CRVLADRDC | (SEQ ID NO:151) | 1/42 | NT | NT |
| CRWSSMIWC | (SEQ ID NO:152) | 1/42 | 7 | 11 |
| CSLQGIVGC | (SEQ ID NO:153) | 1/42 | NT | NT |
| CSRSVSRLC | (SEQ ID NO:154) | 1/42 | NT | NT |
| CVAADRYVC | (SEQ ID NO:155) | 1/42 | NT | NT |
| CYGAWRVSC | (SEQ ID NO:156) | 1/42 | 6 | 14 |

NT, not tested

Phage Binding Assays with rhAngiostatin Selected Clones.

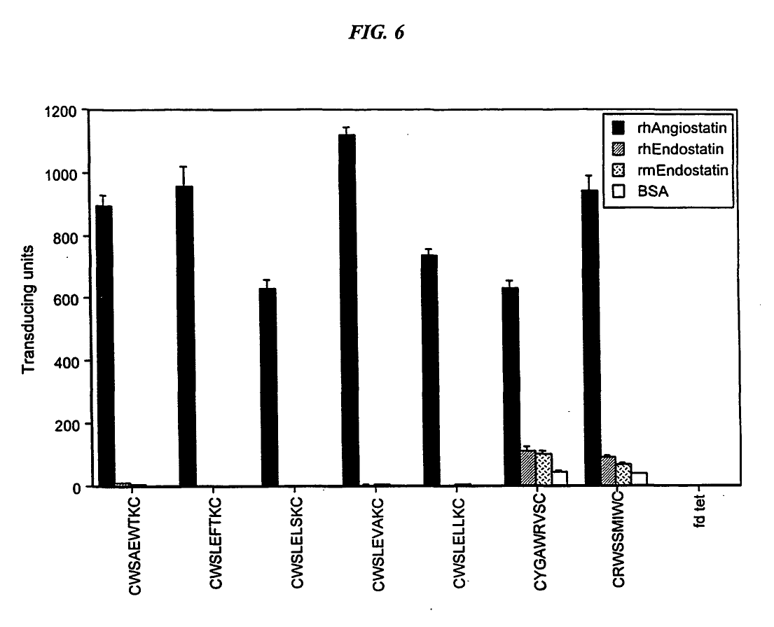

The specificity of binding of selected phage clones was determined (FIG. 6). The most frequently occuring clones, as well as several others, were examined for binding to rh-Angiostatin, rh-Endostatin, rm-Endostatin and BSA, coated on microtiter wells (rh=recombinant human, rm=recombinant mouse protein). As shown in FIG. 6, all phage clones tested that were recovered by panning against rh-Angiostatin were highly selective for angiostatin binding, with only low levels of binding observed against endostatin or BSA. Several of the clones tested showed no observable binding to either endostatin or BSA. It is concluded that highly selective targeting peptide sequences against angiostatin can be obtained by the disclosed methods.

Candidate Proteins for Angiostatin Receptors

Homology searches of the angiostatin targeting peptides were run against databases of known protein sequences. The results show that the peptides displayed by rhAngiostatin-binding phage are similar to certain regions found within ECM (extracellular matrix) proteins, growth factors and cell surface receptors (Table 11).

TABLE 11
Similarity of angiostatin binding peptides with known cell surface
receptors

| | Cell surface | | |
|---|---|---|---|
| Peptides | receptor candidate | % Homology | Region AA |
| CWSLEVAKC | T cell receptor V | 75% (6/8 AA) | 11-18 |
| (SEQ ID NO:145) | beta chain | | |
| CWSAEWTKC | thrombospondin | 70% (7/10 AA) | 382-391 |
| (SEQ ID NO:142) | | | |
| CWSLESLKC | Insulin-like growth | 87% (7/8 AA) | 47-54 |
| | factor I receptor | | |
| (SEQ ID NO:146) | TNF-α | 85% (6/7 AA) | 142-148 |
| | Aminipeptidase N | 71% (5/7 AA) | 226-232 |
| | (CD13) | | |
| | Down Syndrome | 83% (5/6 AA) | 1438-1443 |
| | Cell Adhesion | | |
| | Molecule Precursor | | |
| CRVLADRDC | Melatonin-related | 100% (6/6 AA) | 89-94 |
| (SEQ ID NO:151) | receptor (H9) | | |
| CRWSSMIWC | Hepatocyte growth | 100% (5/5 AA) | 152-156 |
| (SEQ ID NO:152) | factor precursor | | |
| | Tubulin | 83% (5/6 AA) | 397-402 |
| | WNT-16 Protein | 66% (4/6 AA) | 80-94 |
| | precursor | | |
| CYGAWRVSC | Thrombospondin 1 | 83% (5/6 AA) | 241-246 |
| (SEQ ID NO:156) | | | |

Protein-Protein Interaction

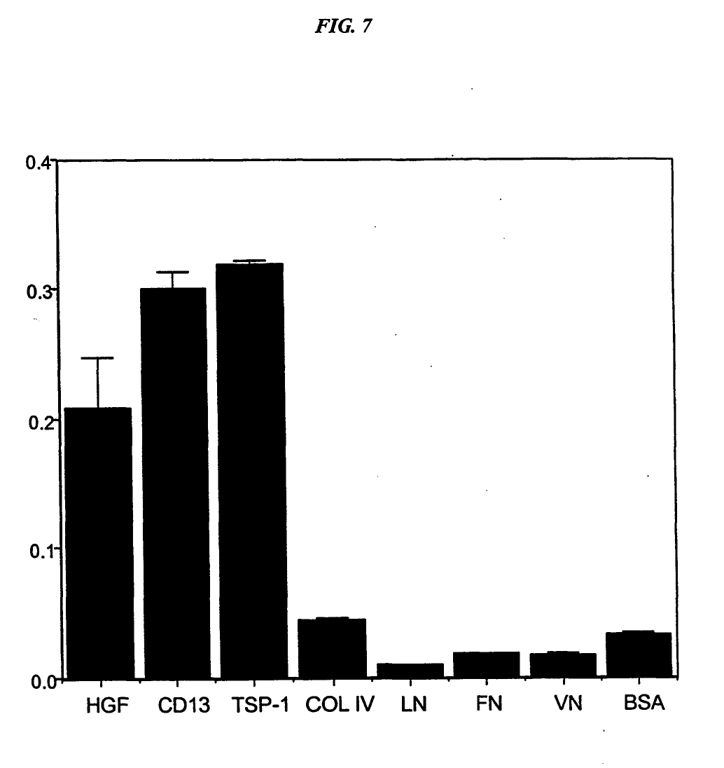

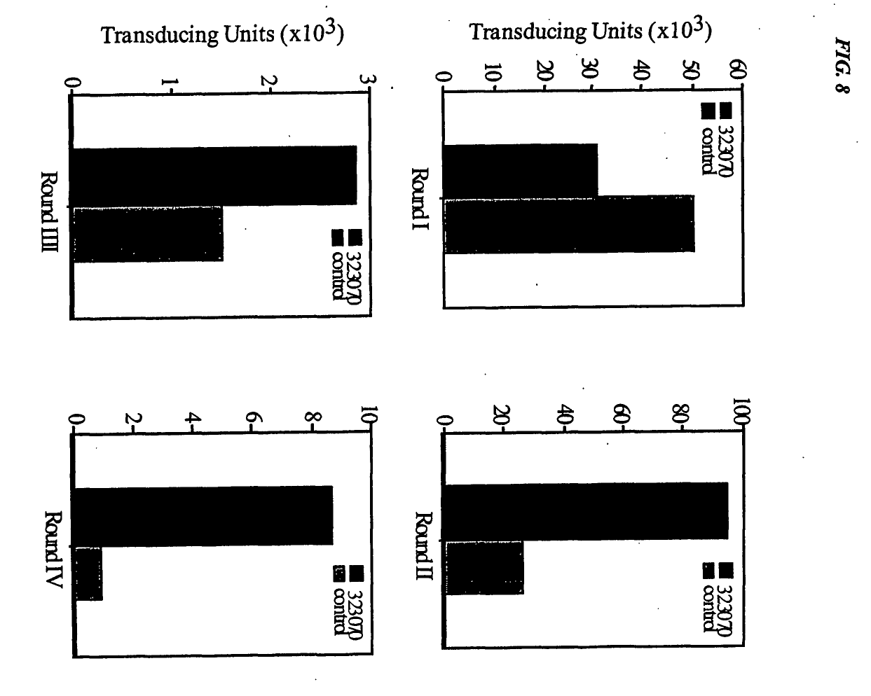

The biological relevance of the homology searches performed above was examined by determining whether any of the identified homologous proteins bind to angiostatin. HGF (hepatocyte growth factor) and CD13 (Aminopeptidase N) were selected as examples of proteins with homology to angiostatin targeting peptides. Thrombospondin 1 (TSP-1) was used as a positive control, since it is known to bind to collagen XVIII on the sequence containing angiostatin (Silverstein, 1984). As shown in FIG. 7, angiostatin binds to HGF, CD13 and TSP-1, but not to other ECM proteins such as COL IV (collagen IV), LN (laminin), FN (fibronectin), or VN (vitronectin) (FIG. 7). These results showed that proteins identified by homology with ligand targeting peptides are good candidates to be the natural receptors for those ligands.

Binding of angiostatin to CD13 was competitive with the NGR peptide (Burg et al., 1999) in a dose-dependent manner (not shown). Control experiments were run with the CARAC peptide (SEQ ID NO: 169) (not shown). Binding of angiostatin to CD13 was inhibited by more than 50% by 0.5 μg of NGR peptide, while binding was unaffected by up to 500 μg of CARAC peptide (SEQ ID NO: 169) (not shown).

The skilled artisan will realize that the targeting peptide sequences identified herein are of potential use for the development of novel agents of potential use as anti-angiogenic or pro-angiogenic activity. Further, the putative angiostatin receptor proteins identified by homology to the angiostatin targeting peptides are potential targets for therapeutic treatment directed towards anti-angiogenesis or pro-angiogenesis. Anti-angiogenic agents of therapeutic use for tumor treatment are also within the scope of the present invention.

### Example 7

Identifying a Novel Viral Agent in Hodgkin's Disease by Phage Display

Studies of Hodgkin's disease (HD) have suggested that HD is associated with infection by type 1 Epstein-Barr virus (EBV) (Weiss et al., 1987; Herbst et al., 1991; Jarrett et al., 1996; Razzaque et al., 1996). The incidence of EBV in HD patients ranges from 40-50% in developed countries and up to 94% in developing countries (Chang and Weiss, 1996). The incidence of HD with EBV seropositivity also varies with histological subtype, being about 80-90% in mixed cellularity (HDMC) and 40-50% in nodular sclerosis (HDNS) (Lyons and liebowitz, 1998). Taken together, these data suggest a strong association between HD and EBV infection. However, a significant fraction of HD cases have no evidence of EBV infection. This is in marked contrast to Burkitt's lymphoma (BL) in which EBV seropositivity is universal (Chang and Weiss, 1996).

In EBV-positive HD, EBV DNA is localized to nearly all RS (Reed Sternberg) cells (Brousset et al., 1991; Herbst et al., 1991, 1992; Pallesen et al., 1991; Weiss et al., 1991). A universal causative role for EBV in HD is questionable, given that about half of HD patients are EBV-negative. Moreover, no association was found between EBV seropositivity and the presence of EBV in H—RS cells (Enblad et al., 1997). Pathological studies of EBV-negative HD patients show previous viral infection since giant multinucleated cells are routinely detected. A possible explanation of these observations is that EBV uses a “hit-and-run” strategy. An equally plausible explanation is that an as yet unidentified viral agent is responsible for the etiology and pathogenesis of HD (Chang and Weiss, 1996; Jarret and MacKenzie, 1999; Staratschek-Jox et al., 2000).

An unbiased method of searching for a novel viral epitope(s) amongst HD sera was used to select phage display peptide libraries against the pool of circulating immunoglobulins of HD patients. A potential by-product of this research is the identification of such viral epitopes.

Phage display has been used to identify peptide epitopes from random peptide libraries in viral diseases such as hepatitis C (Pereboeva et al., 1998, Prezzi et al., 1996) and measles (Owens et al., 2000). Identification of viral and tumor cell surface epitopes has the potential to be clinically useful for developing neutralizing antibodies that may protect against viral infections and for developing serodiagnostic testing (Pereboeva et al., 2000).

Methods

Purification of HD and Control IgGs

IgGs were isolated from the serum of HD patients by batch binding to Protein G agarose, using Pierce ImmunoPure Immobilized Protein G binding buffer. The bound IgGs were eluted with a Protein G elution buffer (Pierce) and immediately neutralized with 0.1 volume of 1 M Tris-Cl, pH 9. After identification of targeting peptides as described below, purified IgGs were incubated with the glutathione Sepharose 4B-bound GST-fusion proteins to affinity purify the specific IgGs which recognized the corresponding targeting peptide epitopes. The glutathione Sepharose 4B resin was pelleted by centrifugation, and rinsed to remove non-specific binding IgGs. IgGs were eluted with acidic elution buffer and immediately neutralized with 0.1 volume 1 M Tris-Cl, pH 9.

Subcloning, Expression and Purification of GST-Fusion Proteins

Peptide coding sequences from selected phage were amplified by PCR using forward and reverse primers containing BamHI and EcoRI sites, respectively. The ameplified sequences were cloned into the BamHI-EcoRI site of the GST vector, pGEX-2TK (Amersham/Pharmacia), and the presence of the inserted sequences was verified by sequence analysis.

Positive clones were transformed into a bacterial expression host strain, BL21(DE3)pLysS (Stratagene) and expression of the GST-fusion proteins induced with 200 μM IPTG. The GST-fusion proteins were affinity purified from bacterial lysates by affinity chromatography to immobilized glutathione using established protocols (Smith and Johnson, 1988). Briefly, the GST-fusion proteins were batch-bound to glutathione Sepharose 4B beads, and the resin extensively rinsed to remove non-specific proteins. GST-fusion proteins were eluted by incubating the resin with an excess of reduced glutathione, followed by extensive dialysis of the eluted protein against phosphate buffered saline, pH 7.4 (PBS) to remove the glutathione. To purify epitope-specific HD IgGs, the GST-fusion proteins were not eluted from the solid support as described below.

Immunohistochemistry

Cryostat samples were processed using published methods (Bielenberg et al., 1999). Briefly, samples were fixed with cold acetone, acetone:chloroform (1:1) at RT, and acetone at RT for 5 minutes each, rinsed 3× with PBS, blocked for 20 minutes at RT with PBS supplemented with 5% normal horse serum, and incubated with affinity purified primary human IgG overnight at 4° C. Fixed tissues were rinsed 3× with PBS, blocked in 5% horse serum/PBS for 10 minutes, and incubated in mouse anti-human IgG secondary antibody conjugated to flurochrome dyes such as Cyanine 3 or Cyanine 5 (Amersham/Pharmacia) for 1 hour at RT, and rinsed in PBS. Fluorescence was detected using an Olympus IX70 Inverted microscope fitted with an inverted reflected light fluorescence attachment. If the fluorescence signals were too low to visually detect, the signals were quantitated by laser scanning cytometry (Grace et al., 1999).

ELISA Assay

The affinity GST-fusion proteins were used to screen a collection of HD patient sera by enzyme linked immunosorbent assay (ELISA). The purified GST-fusion proteins were used to coat a 96-well plate at 100 ng/well at room temperature (RT) or at 4° C. overnight. Following coating, the wells were emptied, rinsed, and non-specific sites were blocked with 200 μl 3% BSA/PBS at RT for 1-2 hours. HD sera were applied to each coated and blocked well at 1:100 dilution and then incubated at RT for 1 hour. The wells were rinsed 3× with 3% BSA/PBS containing 0.01% Tween 20, and then incubated for 1 hour with 50 μl each of anti-human alkaline phosphatase conjugated antibody at 1:2000 dilution. Signals were detected in the presence of p-nitrophenyl phosphate by measuring OD₄₀₅ at specific intervals to follow the course of color development. A positive control was the HD sera the peptide was identified from, and a negative control was EBV negative normal sera or BSA.

Choice of Phage Peptide Library.

A CX₇C phage display library in the fUSE5 vector was generated as described above. Bulk amplification was used between each selection round to elute phage.

Overall Strategy

IgGs were isolated from the serum of HD patients using established methods. A phage display random peptide library was screened on this pool of IgGs (Smith, 1985) in a two-step procedure. First, the peptide library was pre-cleared on a pool of IgGs from control normal serum. This step removed nonspecific peptide interactions. Second, the pre-cleared peptide library was screened on the pool of IgGs from the serum of HD patients. This step selected specific interactions between peptides and HD IgGs.

In brief, HD IgGs bound to Protein G agarose were incubated with a Cx₇C phage peptide library that had been pre-cleared on IgGs isolated from non-HD sera. The resultant HD IgG-bound phage were eluted from the solid support, neutralized immediately, and used to infect E. coli strain K91. The phage were amplified and precipitated for a subsequent round of panning (Koivunen et al., 1999). Phage infected K91 were plated onto tetracycline LB agar plates and individual clones were subjected to colony PCR and sequence analysis.

Results

A representative experiment after four rounds of selection on HD IgG is shown (FIG. 8). The figure shows that the selectivity of the targeting phage improves with each round of selection, compared to control phage without insert.

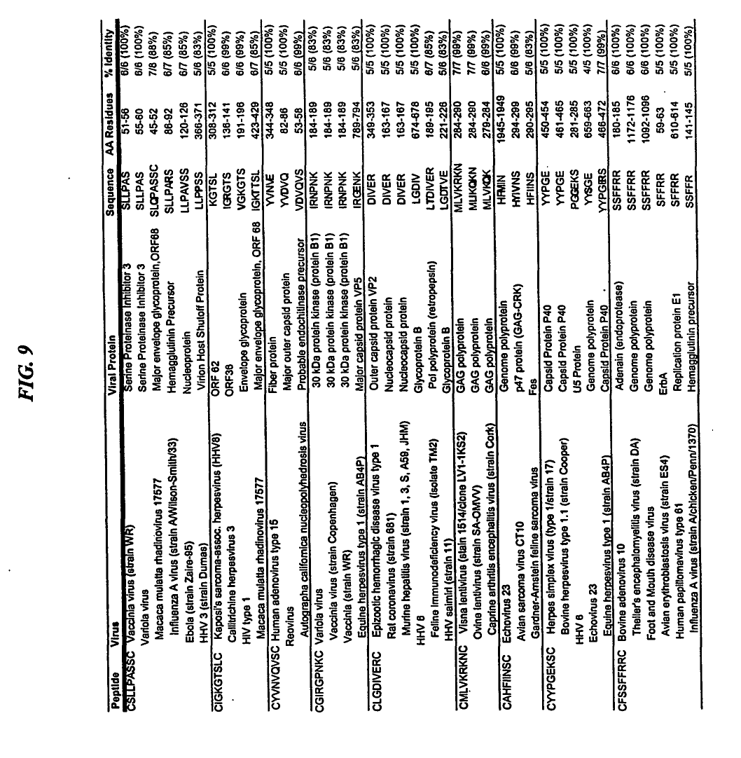

Using the methods disclosed above, a panel of 256 HD phage that were selected against HD sera were sequenced. Each of these peptide sequences was compared to the Swiss Protein database of known proteins to search for amino acid identity. FIG. 9 shows selected viral motifs that were identified. A number of similar proteins from a variety of viruses were aligned using the LALIGN program from the ExPASY website to identify conserved regions. The aligned sequences were also used to search unique epitopes by cross-referencing the peptide sequences in FIG. 9, using the MacVector software package (Oxford Molecular Group). This analysis confirmed that similar viral capsid proteins contain conserved regions within their primary structure.

A short peptide database and analysis software were developed in collaboration with Kim-Ahn Do, Ph.D. (Department of Biostatistics, UTMDACC). This biostatistical program (available on request from the M. D. Anderson Cancer Center) determines the frequency by which any combination of 3 or more amino acid residues occurs. It was found that the conserved regions identified from the LALIGN program matched frequently occurring amino acid sequences identified from the biostatistical analysis. Conversely, unique epitopes occurred less frequently and corresponded to variable regions of the viral proteins analyzed.

The results of this search showed the peptides exhibited sequence identity to a number of viral proteins from a variety of species. Nine HD-targeting peptide sequences were identified from screening IgGs from two EBV-negative HD patients and one EBV-positive HD patient. The targeting peptide sequences identified were CSLLPASSC (SEQ ID NO: 157), CIGKGTSLC (SEQ ID NO: 158), CYVNVQVSC (SEQ ID NO: 159), CLGDIVERC (SEQ ID NO: 160), CMLVKRKNC (SEQ ID NO: 161) CAHFIINSC (SEQ ID NO: 162), CYYPGEKSC (SEQ ID NO: 163), CFSSFFRCC (SEQ ID NO: 164) and CGIRGPNKC (SEQ ID NO: 165). Screening the same peptide library on IgGs from other human solid tumors did not yield peptides sequences that shared sequence identity to known viral proteins (data not shown). Moreover, the same peptides were selected in different HD patients but not in controls.

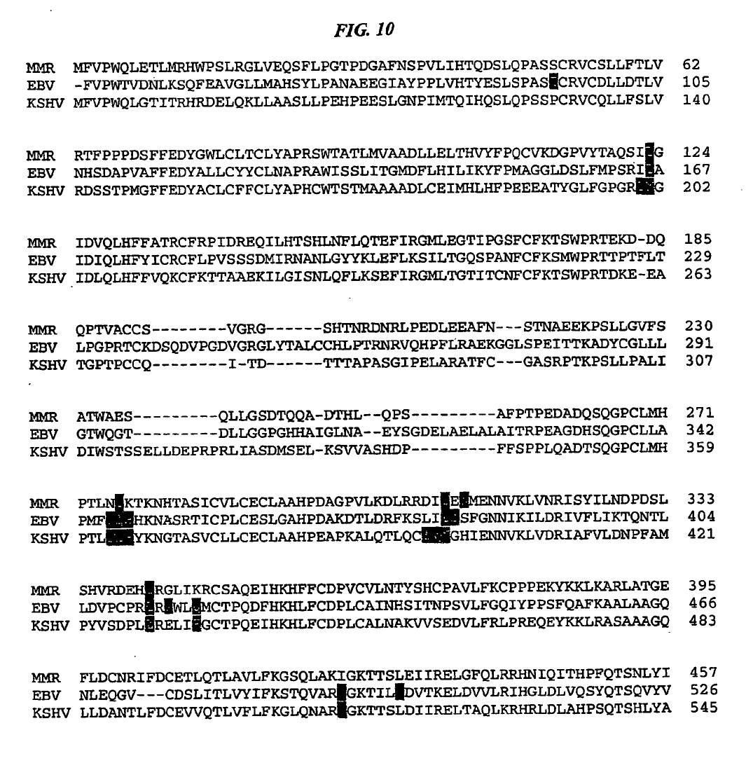

Eight of the nine peptides map to various regions of the major envelope glycoprotein from Macaca mulatta rhadinovirus, KSHV (Kaposi's sarcoma-associated virus) and EBV (Epstein Barr virus) (FIG. 10). The sequence identity between the protein from M. mulatta rhadinovirus, EBV, and KSHV is 35.5% in a 484 amino acid overlap, and 48% in a 467 amino acid overlap, respectively (not shown).

Viruses such as the human papilloma virus 16, hepatitis B and C, Kaposi's sarcoma-associated virus (KSHV), and the Epstein Barr virus (EBV) are recognized to cause a variety of human cancers. Although tumors are known to elicit an immune response against mutated, altered, or overexpressed antigens, a limited number of immunogenic tumor antigens have been thus far identified. The work here illustrates how phage display technology can be used to screen immunoglobulins (IgGs) from Hodgkin's disease (HD) patients to search for novel viral etiological agents.

The present example shows that phage display can be applied to distinguish unique epitopes from IgGs isolated from HD sera in an unbiased fashion. The peptides identified from these studies may be of use for identifying novel viral agents in EBV-negative HD patients. The clinical applications of this work range from the development of vaccines and/or anti-idiotype antibodies for immunotherapy, increased accuracy for diagnostic/prognostic testing, and directed specificity in tumor targeting.

### Example 8

A New Generation of Targeted Phage-Based Vectors for Systemic Gene Delivery in Humans

The compositions and methods disclosed herein are of use for targeted delivery of therapeutic agents to selected organs, tissues or cell types, including cancer cells. In certain embodiments, the targeted therapeutic agent is an expression vector. The present example discloses a non-limiting embodiment illustrating the use. of targeting peptides for delivery of novel expression vectors encoding therapeutic proteins or peptides.

The development of vectors for systemic targeted delivery is required for successful gene therapy. Many groups have developed concepts to target gene therapy vectors, either by using specific promoters or by altering the vector's receptor tropism. Retargeting adenoviral and adeno-associated viral vectors to alternative receptors has been attempted with bispecific adapter molecules that redirect these vectors to growth factor receptors (Douglas et al., .1996; Goldman et al., 1997; Watkins et al., 1997; Miller et al., 1998), CD3 (Wickham et al., 1997a), α integrins and heparan sulfate receptors (Wickham et al., 1996a). Heterologous ligands also have been incorporated into the envelopes of retroviruses or the capsids of adenoviruses and adeno-associated viruses, thereby targeting these vectors to integrins (Dmitriev et al., 1998; Vigne et al., 1993; Girod et al., 1999), T-cell receptors (Engelstadter et al., 2000) or melanoma-associated antigens (Martin et al., 1999). Thus far, a major drawback of these approaches has been that the expression of the receptors is not restricted to the target tissues.

Many malignant, cardiovascular, and inflammatory diseases have a marked angiogenic component. In cancer, tumor vasculature is a suitable target for intervention because the vascular endothelium is composed of non-malignant cells that are genetically stable but epigenetically diverse. In vivo phage display has been used to isolate probes that home selectively to different vascular beds and target receptors expressed only on certain blood vessels. Both tissue-specific (Rajotte et al., 1998; Rajotte and Ruoslahti, 1999) and angiogenesis-related (Pasqualini et al., 1997; Arap et al., 1998; Koivunen et al., 1999; Pasqualini et al., 2000a) vascular ligand-receptor pairs have been identified with this technology (Pasqualini et al., 2000b).

Targeted delivery of cytotoxic drugs, proapoptotic peptides, fluorophores, or cytokines to the vasculature generally improved selectivity and/or therapeutic windows in animal models. Vascular receptors are attractive targets for systemic delivery of gene therapy, in particular because such receptors are readily accessible through the circulation and often can mediate internalization of ligands by cells. While incorporation of vascular homing peptides derived from in vivo phage display screenings into viral vectors has been attempted, this strategy has proven quite challenging because the structure of the capsid and the targeting properties of the peptides can be adversely affected (Wickham, 2000).

Recent data show that gene expression in mammalian cells is possible if phage vectors are processed in the correct trafficking pathway (Kassner et al., 1999; Larocca et al., 1999; Ivanenkov et al., 1999a, 1999b; Poul and Marks, 1999). In theory, phage vectors have several advantages over mammalian viruses conventionally used for gene therapy. Receptors for prokaryotic viruses such as untargeted (wild-type) phage are not expressed on mammalian cells (Id.). Receptor-mediated internalization by mammalian cells can occur if re-targeted phage vectors display certain peptide ligands (Larocca et al., 1999). There is substantial evidence suggesting that phage can be safely administered to patients, as bacteriophage were given to humans during the pre-antibiotic era with no adverse effects (Barrow and Soothill, 1997. Because homing phage have been pre-selected to home to vascular receptors in an in vivo screening, there is no need for further targeting modifications. The localization of gene expression in vivo recapitulates previous observations using immunohistochemistry for phage localization (Rajotte et al., 1998; Rajotte and Ruoslahti, 1999; Pasqualini et al., 1997; Arap et al., 1998; Koivunen et al., 1999). The parental tumor-homing phage used here are known to target receptors expressed in the activated blood vessels of multiple types of human and murine tumors, including carcinomas, melanomas, and sarcomas in mouse models (Pasqualini et al., 1997; Arap et al., 1998; Koivunen et al., 1999). The lung-homing phage and its corresponding receptor expressed in the lung vasculature have also been characterized in mice (Rajotte et al., 1998; Rajotte and Ruoslahti, 1999).

Based on the above properties, targeted systemic gene delivery to the vascular endothelium may be feasible using phage particles homing to cell surface receptors on blood vessels to provide selective tissue expression and adequate vector uptake into the targeted tissue. The present example demonstrates the feasibility of this approach.

Using the claimed methods and compositions, a new generation of phage-based gene delivery vectors is provided that targets the molecular diversity of the vascular endothelium in vivo. The present example shows that targeted phage vectors can promote gene expression in mammalian cells in vitro following specific receptor-mediated internalization. Systemic targeted tissue-specific transduction of the lungs in immunocompetent mice and angiogenesis-related transduction of tumors in immunodeficient mice bearing human tumor xenografts is demonstrated. After genetic cis-elements of adeno-associated virus (AAV) were introduced into a targeted phage backbone, the chimeric vectors markedly increased and prolonged transduction without loss of targeting properties. The term adeno-associated phage (AAP) is proposed for this new class of targeted vectors.

AAP vectors appear to combine favorable biological features of both prokaryotic viruses (peptide display system for receptor targeting and high production yield in host bacteria) and mammalian viruses (long-term transduction stability) and may be of use for systemic gene therapy targeting applications in vivo.

Materials and Methods

Reagents, Cells, and Tissue Culture.

All of the restriction enzymes (New England Biolabs, Beverly, Mass.), T4 DNA ligase (Roche, Indianapolis, Ind.), topotecan (Sigma Chemical Company, St. Louis, Mo.), and cisplatin (Sigma) were obtained commercially. The fMCS1 plasmid was obtained from Dr. George P. Smith (University of Missouri, Mo.). DNA sequence analysis was performed with the Big Dye® terminator sequence kit (Perkin Elmer/ABI Systems, Norwalk, Conn.). All peptides used in this study were synthesized at greater than 95% purity, cyclized, and analyzed by HPLC and mass spectrometry (AnaSpec, San Jose, Calif.). The following human cell lines were used: Kaposi's sarcoma (termed KS1767), 293 embryonic kidney (ATCC; Manassas, Va.), and MDA-MB435 breast carcinoma. Cell lines were maintained in minimal essential medium (MEM; Irvine Scientific, Santa Ana, Calif.) supplemented with 10% fetal calf serum (FCS; Gibco-BRL, Rockville, Md.) plus sodium pyruvate, L-glutamine, and penicillin/streptomycin (Gibco-BRL).

Construction of Phage-Based Targeted Expression Vectors

A fUSE5-based filamentous phage display vector was modified to transduce mammalian cells with the β-galactosidase reporter gene under the control of the cytomegalovirus (CMV) immediate early gene promoter. The sequences for the targeting peptides were inserted into the Sfi I sites of the fUSE5 phage display cloning vector of the gene III coat protein (pIII). The RGD-4C targeting peptide (CDCRGDCFC, SEQ ID NO: 166) was chosen for its ability to bind to and be internalized by αv3/5 integrin expressing cells in tumor vasculature. A second tumor homing targeting peptide, the MMP-2/9 binding HWGF peptide (CTTHWGFTLC, SEQ ID NO: 167) was also selected. As a negative control vector, an fd-tet phage derived construct that carries the CMV-β-gal cassette but does not display a targeting peptide was used.

Targeted RGD4C-β-gal phage vector was engineered in a two-step process that included the generation of an intermediate construct (termed RGD-4C-fMCS1) and subsequent production of RGD-4C-β-gal. RGD-4C-fMCS1 contained the oligonucleotide insert encoding the RGD-4C targeting peptide (SEQ ID NO: 166) and a fragment of the fMCS1 plasmid that had a multicloning site (MCS) for insertion of transgenes. RGD-4C phage-derived fUSE5 DNA (Koivunen et al., 1995) and fd-tet phagederived fMCS1 DNA were purified from lysates of host bacteria (E. coli MC1061).

The intermediate RGD-4C-fMCS1 vector was prepared by ligating a 5.4-kb BamHI/SacII fragment of the RGD-4C plasmid to the 4.1 kb BamHI/SacII fragment of the fMCS1 plasmid. A 14 kb RGD-4C-β-gal phage plasmid was obtained by insertion of a 4.5 kb PstI CMV-β-gal fragment derived from pCMVβ (Clontech, Palo Alto, Calif.) into the PstI site of RGD-4C-fMCS1. This allowed cloning of the CMV-β-gal cassette in either forward or reverse orientation. Orientations of resulting vectors were differentiated by EcoRV restriction analysis and by DNA sequencing. Targeted phage vectors were designated fRGD4C-β-gal (forward) and rRGD4C-β-gal (reverse). Other targeting (HWGF-β-gal, GFE-β-gal) phage and insertless control (fd-β-gal) phage were constructed using the same methods. GFE phage contained the lung targeting peptide GFE-1 (CGFECVRQCPERC, SEQ ID NO: 168).

A targeted phage/AAV chimeric vector was produced by cloning a 2.8 kb fragment of pAAV-eGFP (enhanced GFP; Stratagene) from ITR to ITR into the PstI site of RGD-fMSC. Briefly, pAAV was digested with PacI to release a 2.8 kb fragment, which was blunted with DNA polymerase and cloned into the blunted PstI site of RGD-fMSC (thus destroying the PstI restriction site). In each of the constructs, correct orientation of insert was verified by restriction analysis. Single clones in each orientation were sequenced. Unless otherwise stated, the forward vectors were used in this example.

Phage DNA Transfection into Mammalian Cells

The double-stranded DNAs of the replicative forms of targeted (RGD4C-β-gal, HWGF-β-gal, GFE-β-gal) and insertless control (fd-β-gal) constructs were prepared using a Plasmid Maxi kit (Qiagen). The single-stranded DNAs of the infective forms of the phage vectors were extracted from the phage capsid proteins using Strataclean resin (Stratagene), followed by two ethanol precipitations. DNA was quantified by spectrophotometry with 1.0 A₂₆₀ equal to 40 μg/ml for single-stranded DNA or 50 μg/ml for double-stranded DNA. The 293 recipient cells were transfected with 5 μg of either double-stranded or single-stranded phage DNA into 5×10⁵ cells, using the SuperFect® reagent (Qiagen) according to the manufacture's protocol. Both the gene expression and enzyme activity of β-gal were evaluated at least 48 hours post-transfection. Cells were incubated with the X-gal substrate for 3 hours at 37° C. and enzyme activity was visualized by using an in situ β-galactosidase staining kit (Stratagene) according to the manufacturer's instructions.

Vector Production, Purification, and Titration

Phage vectors were isolated and purified from the culture supernatant as disclosed (Pasqualini et al., 2000b; Smith and Scott, 1993). Phage were re-suspended in Tris-buffered saline (pH 7.4) and re-centrifuged to remove residual bacteria and debris. The resulting supernatant containing the phage in suspension was filtered through a 0.45 μm filter and titered according to standard protocols (Id.).

Targeted Phage Vector Transduction and Specific Inhibition using Synthetic Peptides

MDA-MB435 and KS1767 cells were cultured on 8-well chamber glass slides. The culture media was replaced by 200 μl of MEM with 2% FCS and 5×10¹⁰ TU of RGD-4C-β-gal, HWGF-β-gal, or fd-β-gal phage vectors (at 10⁵ transducing units/cell in each case). Phage were incubated with cells for 3 hr at 37° C., followed by a medium change to MEM plus 10% FCS. The cells were incubated for 72 hr at 37° C. to allow for β-gal gene expression.

In the peptide inhibition experiments, MDA-MB435 cells were cultured on 12-well plates and then incubated with 10 μg of RGD-4C peptide (SEQ ID NO: 166) or control peptides (CARAC, SEQ ID NO: 169 or CKDRFERC, SEQ ID. NO: 59) in normal growth media for 30 minutes. KS1767 cells were grown on 12-well plates and then incubated with 40 μg CTTHWGFTLC (SEQ D NO: 167) or control peptides in normal growth media for 30 minutes. After this the growth media were replaced by 500 μl of MEM containing 2% FCS and 5×10¹⁰ transducing units (TU) of either RGD-4C-β-gal, HWGF-β-gal, or control fd-β-gal phage. Phage vectors were incubated on peptide-treated cells (three hours at 37° C., 5% CO₂) followed by a media change to MEM plus 10% FCS. Transduced cells were then maintained in a cell incubator for 72 hours (37° C., 5% CO₂).

In the cell culture transduction assay, β-gal expression was analyzed by immunofluorescence studies. For quantification of expression in cell culture, the transduced cells were washed with PBS and permeabilized with 0.2% Triton X-100 for five minutes on ice, followed by blocking with 1% BSA in PBS. An anti-β-gal antibody (Sigma) diluted to 1:2,000 in blocking solution was then incubated with the cells overnight. Next, a Texas Red-conjugated secondary antibody (Caltag, Burlingame, Calif.) diluted to 1:600 in PBS was incubated with the cells for 1 hour. The degree of β-gal gene expression was determined by counting fluorescent cells in at least ten fields under an inverted microscope (Nikon, Japan). Quantification of the β-gal activity in cell culture was measured as relative light units (RLU) in a luminometer and then normalized to the amount of protein in micrograms, as determined by the Lowry method in a protein assay kit (Bio-Rad Protein Assay®; Hercules, Calif.). Subsequently, blue cells were counted under an inverted microscope (Nikon).

In the peptide inhibition assays, β-gal activity in cell lysates was detected by the Galacto-Star® chemiluminescent reporter gene system (Tropix, Bedford, Mass.) according to the manufacturer's protocol. In other peptide inhibition assays, 293 cells were plated at 3×10⁵ cells/well and incubated with either 1 mg/ml of RGD-4C peptide (SEQ ID NO: 166) or irrelevant control peptides (CARAC, SEQ ID NO: 169 or CKDRFERC, SEQ ID NO: 59). After 30 minutes, cells were washed and 10⁵ TU of phage per cell were added for 4 hours in serum free media. After the 4 hours, 10% FCS supplemented medium was added. Cells were analyzed for GFP gene expression at 72 hours post infection. For GFP detection, cells were analyzed by fluorescence activated cell sorting (FACS) in a FACScan (Becton-Dickinson, San Jose, Calif.) or counted and photographed under a fluorescence microscope (Nikon).

For time course of gene expression assays, cells were plated at 3×10⁵ cells/well and infected with 10⁵ TU of phage per cell for 4 hours in serum-free media. After 4 hours, 10% FCS supplemented medium was added. Cells were visualized 72 hours post-infection and sorted by FACS for GFP expression 7 days after infection. GFP-positive cells were plated in T75 tissue culture flasks and serial assays of GFP expression as described above were made weekly for the next 60 days.

Genotoxic Agents

Semi-confluent cells were infected with 10⁵ TU of phage per cell for 4 hours in serum free media, after which fresh medium supplemented with FCS was added (no phage were washed out or removed). In some experiments, a phage admixture of forward and reverse clones at 10¹⁰ TU (forward/reverse molar ratio=1) was tested. Next, cells were incubated for 36 hours followed by the addition of genotoxic drugs (topotecan, 10 μM; cisplatin, 10 μM) or administration of UV radiation (15 J/m²) with a cross-linker apparatus (UV Stratalinker Model 2400; Stratagene). At 72 hours post-infection, the cells were analyzed for transduction of a reporter gene (β-gal or GFP), and gene expression was normalized per cell number relative to controls.

In vivo Transduction of Tumor Xenografts and Normal Lung in Mouse Models

Female 4-month old nude mice and female 4-month old immunocompetent C57B1/6 mice (Harlan Sprague Dawley, San Diego, Calif.) were used. Avertin (0.015 ml/g) was used as an anesthetic. Tumor xenografts derived from human Kaposi's sarcoma KS1767 cells were established by injecting tumor cells (10⁶ cells per mouse in 200 μl of serum-free MEM) into the mammary fat pad of nude mice. Tumor-bearing mice with matched tumor sizes were used for systemic gene transfer experiments 20 to 40 days afterwards when tumors reached 0.5 to 1.5 cm in diameter. In tumor transduction experiments, RGD-4C-β-gal, HWGF-β-gal, and fd-β-gal phage (10⁹ TU/mouse) were injected intravenously (tail vein) into female nude mice carrying subcutaneous tumor xenografts. One week after vector administration of the targeted or control phage, tumors and control organs were surgically harvested under deep anesthesia and the mice euthanized. β-gal expression in the tumor and control tissues was detected by an anti-β-gal antibody by using a peroxidase-based immunodetection kit (Vector Labs, Burlingame, Calif.). In lung transduction experiments, GFE-β-gal phage and fd-β-gal control phage (10⁹ TU/mouse) were injected intravenously into female C57B1/6 mice. Lungs and livers were harvested two weeks after vector administration. For in vivo experiments involving tissue extracts, β-gal activity in the lung and control tissues were detected by a chemiluminescent assay system (Tropix).

Results

Targeted Phage Vectors Designed to Drive Gene Expression in Eukaryotic Cells

The fUSE5-based filamentous phage display vector was modified by inserting a β-galactosidase (β-gal)-encoding gene under the control of a CMV promoter into an intergenic region of the phage genome to construct a fUSE5-β-gal backbone vector. DNA olignonucleotide sequences encoding the targeting peptides CDCRGDCFC (SEQ ID NO: 166, referred to as RGD4C) (Pasqualini et al., 1997; Arap et al., 1998), CTTHWGFTLC (SEQ ID NO: 167, referred to as HWGF) (Koivunen et al., 1999), and CGFECVRQCPERC (SEQ ID NO: 168, referred to as GFE) (Rajotte et al., 1998; Rajotte and Ruoslahti, 1999) were inserted into the Sfi I site of the gene III minor coat protein (pIII) of the fUSE5 phage. The resulting viral constructs (RGD4C-β-gal, HWGF-β-gal, and GFE-β-gal) were used for production of targeted phage particles that displayed the targeting peptides on their outer surface and carried a CMV-β-gal reporter gene. RGD-4C-β-gal and HWGP-β-gal were designed to target αv integrins and matrix metalloproteinases (MMP-2 and MMP-9), respectively. Both receptors are expressed in angiogenic vasculature. The GFE-β-gal phage was designed to target membrane dipeptidase (MDP) expressed in lung vasculature. The same strategy was used to construct the other targeting and control vectors.

Phage DNA Context Permits Transgene Expression in Mammalian Cells

To determine whether the inserted β-gal cassette was functional, embryonic human kidney cells were transfected with the infective forms of the phage DNA constructed to contain the reporter transgene in either forward or reverse orientation. A CMV-driven mammalian expression vector was used as a positive control and an empty vector as a negative control for β-gal expression. Transfer of the modified single-stranded DNA of the phage infective form promoted transgene expression in mammalian cells (not shown). The orientation of the transgene cassette did not significantly influence the level of gene expression (not shown). Therefore all subsequent experiments used the vector with the β-gal expression cassette in the forward orientation. Given that single-stranded DNA does not support gene expression in mammalian cells and that the infective forms of the phage genome are single-stranded, these results strongly suggest that the single-stranded phage genome were converted to double-stranded DNA in recipient cells prior to gene expression. It was observed that DNA from replicative forms of the phage, which are double-stranded, expressed the β-gal transgene several fold more efficiently, at levels comparable to the mammalian expression vector used as the positive control (data not shown).

Receptor-Mediated Internalization and Specific Transduction of Recipient Cells by Targeted Phage Vectors in vitro.

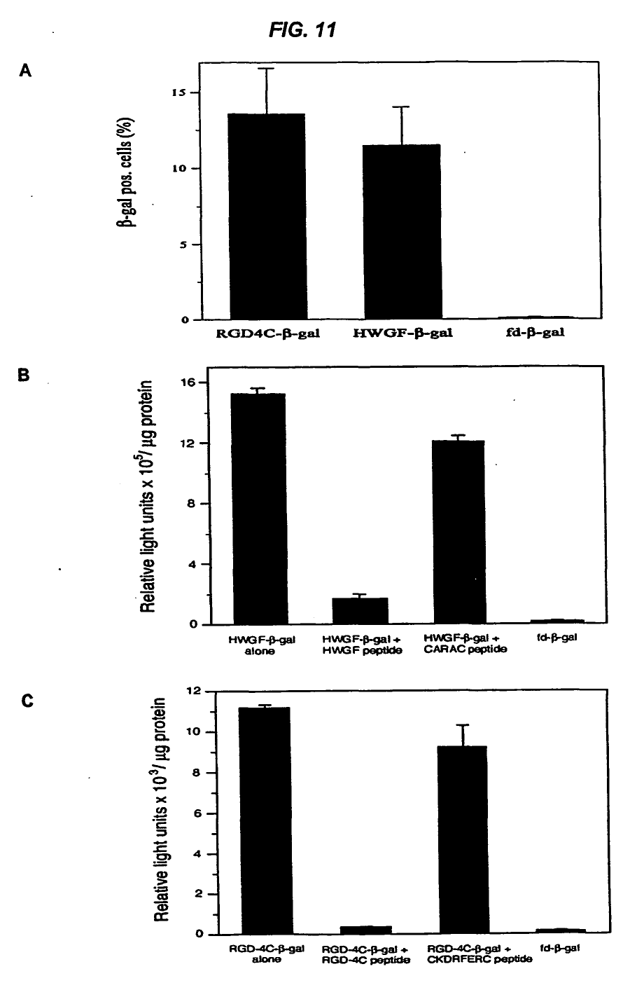

Having shown that the transgene constructs were functional, the transduction of human cell lines expressing receptors targeted by the RGD4C-β-gal and HWGF-β-gal phage vectors was examined. The untargeted fUSE5-derived control phage vector (termed fd-β-gal) was used as a negative control. RGD4C-β-gal phage and HWGF-β-gal phage were incubated with breast cancer and Kaposi's sarcoma cells (MDA-MB-435 and KS1767 lines, respectively). Both cell lines express high levels of the RGD-4C-receptors αvβ3 and αvβ5 integrins (Pasqualini et al., 1996) and of the HWGF receptors MMP-2 and MMP-9 (Koivunen et al., 1999). β-gal transduction of 14±2% (mean±standard error of the mean; SEM) of MDA-MB-435 cells incubated with RGD4C-β-gal phage and 12±2% (mean±SEM) of the KS1767 cells incubated with HGWF-β-gal was observed (FIG. 11). Comparable transduction results were also obtained by incubating HWGF-β-gal on MDA-MB435 cells and RGD4C-β-gal on KS1767 cells (data not shown). Control phage (fd-β-gal) were not internalized when incubated with either cell line (not shown) and only minimal β-gal transduction (˜0.1% of the tumor cells) could be detected (FIG. 11). To demonstrate specificity, the interaction of RGD-4C-β-gal and HWGF-β-gal phage was blocked by pre-incubating the target cells with the corresponding synthetic peptides. In each case, almost complete inhibition of transduction (greater than 99% with RGD4C peptide; greater than 90% with CTTHWGFTLC peptide, SEQ ID NO: 167) was observed in a dose-dependent manner (FIG. 11B, FIG. 11C). Pre-incubation with nonspecific negative control peptides had no significant effects on transduction of the recipient cells (FIG. 11B, FIG. 11C). These data show that transduction of mammalian cells by internalized phage vectors in vitro is substantial, specific, and mediated by ligand-receptor mechanisms.

Targeted Transduction of Tissue-Specific and Tumor Vasculature upon Systemic Administration in vivo

To determine whether the targeted RGD-4Cβ-gal and HWGF-β-gal phage vectors could selectively transduce tumors upon systemic administration, each vector was intravenously injected into nude mice bearing human KS1767 Kaposi's sarcoma xenografts. KS1767 cells are suitable because they form well-vascularized tumors and the receptor expression profiles in tumor cells and tumor-associated blood vessels has been characterized. αv integrins and gelatinases (MMP-2 and -9) are highly expressed on the KS1767-derived tumor xenografts and their angiogenic vasculature. Thus, phage displaying RGD4C and HWGF peptides target KS1767 tumors efficiently and specifically in vivo (Pasqualini et al., 1997; Arap et al., 1998; Koivunen et al., 1999). Tumors and control organs (liver and brain) were surgically harvested one-week after administration of the vectors and immunostained with an anti-β-gal antibody. The RGD4C-β-gal, HWGF-β-gal and control fd-β-gal vectors were analyzed. Strong β-gal immunostaining was observed within tumors, while negligible immunostaining was seen in control organs (not shown). In contrast, tissues recovered from mice that received untargeted negative control fd-β-gal phage vector did not show detectable β-gal expression in either the tumor or the control organs, including liver and brain (not shown). In each case, β-gal reactivity matched the corresponding immunostaining pattern of phage targeting to the vascular endothelium of blood vessels in tumors vivo (Pasqualini et al., 1997; Arap et al., 1998; Koivunen et al., 1999).

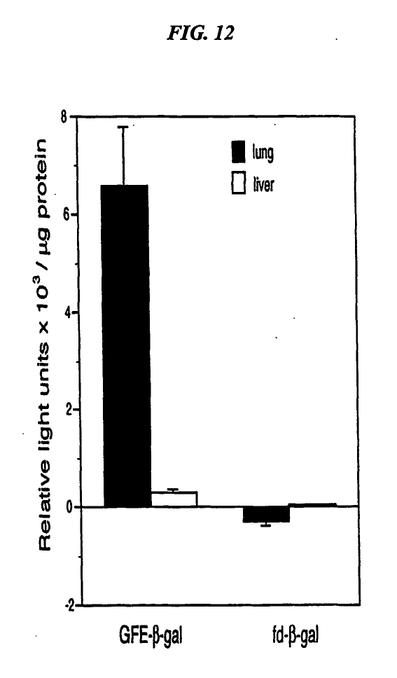

Targeted gene delivery was evaluated in vivo by using GFE-β-gal, a phage vector targeted to MDP in the vascular endothelium of lung blood vessels. The lung-homing GFE-β-gal vector was injected intravenously into immunocompetent C57B1/6 mice. Substantial β-gal activity was seen in the lungs of mice injected with GFE-β-gal phage but not in the lungs of mice injected with fd-β-gal control (FIG. 12). In contrast, the β-gal activity in the liver of mice injected with the GFE-β-gal phage was similar to that of background β-gal activity from mice injected with control phage (FIG. 12). Taken together, these results show the feasibility of in vivo systemic gene delivery and transduction targeted to and mediated by vascular receptors selectively expressed in tumors and in normal organs.

Increase in Transduction by Genetic Trans-Complementation

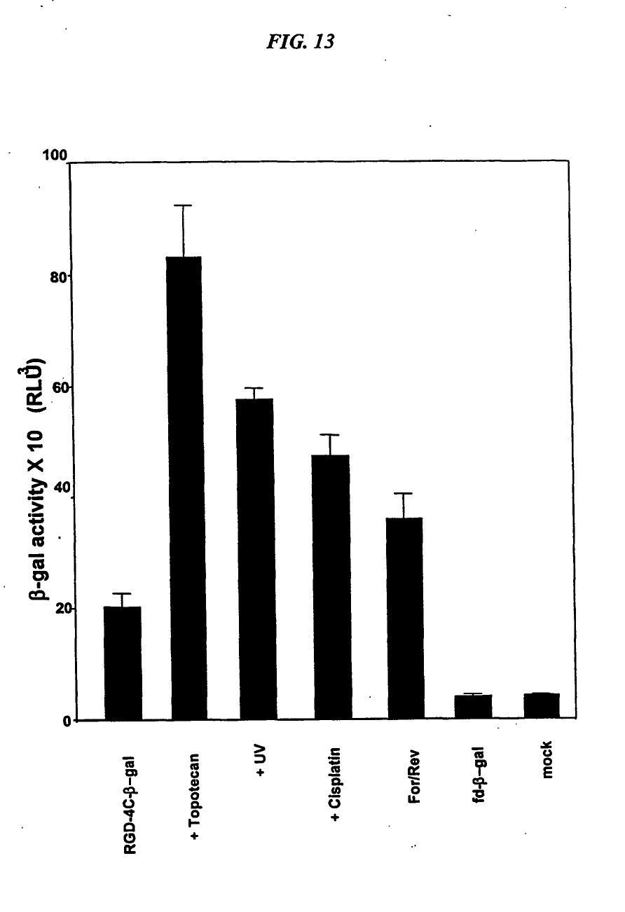

Because the genome from the infective form of M13-derived phage is single-stranded, conversion to double-stranded DNA is required to allow gene expression. Treatment with genotoxic agents that promote unscheduled DNA repair should enhance the transduction of genes carried by single-stranded phage vectors. Cells infected by targeted phage vectors with were challenged with genotoxic agents such as ultraviolet (UV) radiation and cancer chemotherapy drugs (topotecan and cisplatin) (FIG. 13). This approach consistently resulted in gene transduction several fold higher than various controls (FIG. 13). Administering an equal mixture of forward and reverse single-stranded phage clones showed a two-fold increase in gene expression relative to the same molar concentrations of either forward or reverse phage. It appears that the presence of sense and anti-sense of the reporter gene allowed hybridization of the strands to occur; resulting in the formation of double-stranded DNA. The enhancement of gene expression by DNA lesions or genetic trans-complementation indicates that conversion to double-stranded DNA may be a rate-limiting step in developing effective therapeutic phage vectors. These data also suggest the possibility of synergism if cytotoxic agents commonly utilized in clinical applications are used in combination with phage-derived vectors.

Phage/AAV Chimeric Vectors Markedly Improve Gene Transduction Stability

It was determined whether the incorporation of genetic cis-elements derived from AAV (a single-stranded mammalian virus) into targeted phage-based constructs would affect gene transduction. Chimeric vectors composed of a targeted phage and an AAV genome from inverted terminal repeat (ITR) to ITR were produced, with a reporter gene fused to a CMV promoter and a poly A terminator inserted between ITR sequences, and the targeting peptide inserted independently into the phage/AAV chimera. The targeting properties of the resulting chimeric vectors were not altered by insertion of AAV genetic elements. Specific inhibition by the corresponding synthetic peptide was again observed (not shown) indicating that the phage targeting features were intact.

Having established that the tropism of the targeted vector was preserved, the effects of AAV genome insertion on transgene expression were determined. The levels of gene expression remained unchanged (data not shown). However, the duration of gene transduction was markedly prolonged relative to the parental targeted phage (not shown). Robust long-term expression of the reporter gene was seen beyond eight weeks (data not shown). This finding is in clear contrast to the one-week transgene expression usually observed with the parental targeted phage vector. Preliminary studies show that the combination of genotoxic agents plus insertion of AAV cis-elements appears to be at least additive if not synergistic (data not shown).

To rule out the possibility of genetic complementation by trans-acting factors in the permissive 293 cell line, the transduction of HepG2 (liver carcinoma-derived) and MDA-MB-435 cells was examined. Similar gene expression levels and duration of expression were observed (data not shown). Taken together, these data indicate that phage/AAV chimeric vectors may be readily constructed and used with no apparent losses in their targeted acquired tropism and with substantial enhancement in the long-term stability of the genes transduced.

Discussion

This examples provides the first demonstration that systemic gene delivery can be achieved by genetically adapting targeted phage clones selected from screenings of phage display random peptide libraries.

It has been shown that targeting peptides can be integrated into conventional gene therapy vectors and used for organ, tissue or cell type selective delivery. These strategies have been technically challenging but not necessarily efficient. The present example resolves issues of specificity and efficiency by taking advantage of peptide ligands selected from phage libraries in vitro and in vivo. The example demonstrates that the null-tropism of wild-type phage towards mammalian cells can be modified to target and deliver genes to receptors expressed on the vascular endothelium of normal organs (such as the lung) and tumors. Thus, the phage vectors disclosed herein have a number of potential advantages. Their targeting to selective vascular beds is based on receptor expression patterns that are known and characterized. The receptors are accessible to circulating probes. These ligand-receptor pairs provide internalization of the vector into targeted cells.

Despite extensive research on gene transfer into cells with conventional vectors, only a limited number of studies have shown gene transduction in vivo by re-directing the native tropism of a virus (Reynolds et al., 1000; Wickham, 2000; Grifman et al., 2001). While it has been shown that phage can promote gene expression in vitro, gene transduction in vivo after systemic administration of a targeted phage vector has not as yet been reported. A major limitation in the practical use of phage vectors has been poor levels of transduction achieved in vivo. A possible cause of this observation is the low efficiency of conversion from single-stranded to double-stranded DNA occurring in mammalian cells. To solve this problem, two non-mutually exclusive strategies were applied. [1] Enhancement of gene transduction by genotoxic agents (cytotoxic drugs and UV radiation) which cause strand breaks and promote DNA repair. [2] Genetic incorporation of AAV cis-elements into targeted phage vectors. It is tempting to speculate that the incorporation of the inverted terminal repeats from AAV provides stable hairpin loops that facilitate formation of complementary phage DNA strands.

The term adeno-associated phage (AAP) is proposed for the new class of vectors for gene delivery described herein. The biological features of AAP are distinct from either targeted phage or AAV. While the enhanced duration of gene transduction by AAP is similar to the long-term expression patterns associated with AAV transduction, the receptor-mediated targeting is characteristic of phage clones selected in in vivo screenings. Thus, AAP are endowed with several advantages as a gene therapy vector. AAP are easy to produce in high titers in host bacteria. No helper viruses or trans-acting factors are needed. The native tropism of AAV for human cells is eliminated because there is no AAV capsid formation. The AAP vectors are targeted because they incorporate peptides that have been isolated in vivo and are defined by their ability to home to selective vascular beds. Targeted gene delivery specific to the ligand-receptor pair to which the phage is directed was possible, and gene expression was maintained for over two months.

The present example describes a new generation of targeted phage-based vectors that enable systemic gene delivery and robust long-term transgene expression. Novel chimeric phage-based vector containing genetic elements from adeno-associated virus (AAV) have been designed, and tested. These vectors (i) specifically home to receptors that have been well characterized for selective expression on the vascular endothelium, (ii) can deliver genes to angiogenic or tissue-specific blood vessels, and (iii) markedly increase transduction stability and duration of gene expression. These data indicate that targeted phage-based vectors and their derivatives may have potential clinical applications.

### Example 9

In vitro Results with Targeted Phage Delivery

The targeting phage vectors developed in Example 8 above were examined in cell culture experiments.

Methods

Phage Growth, Purification, and Titering

Phage particles were isolated from the E. coli host strain XL1-Blue MR (Stratagene, San Diego. Calif.). The phage particles were purified from the culture supernatant by two precipitations in 0.15 volume polyethylene glycol 8000 (Sigma, St. Louis, Mo.). The phage particles were resuspended in Tris-buffered saline (pH 7.4) and centrifuged to remove any residual bacteria and contaminating debris. The resulting supernatant containing the phage suspension was filtered through a 0.45 μm filter and titered following standard protocols (Smith & Scott, 1993).

Plasmid Preparation for Transfection.

The replicative form of forward and reverse RGD4C-β-gal and HWGF-β-gal plasmids were prepared using the Plasmid Maxi kit (Qiagen). The non-replicative ssDNA phage genome was extracted from the phage capsid proteins using Strataclean resin (Stratagene), followed by ethanol precipitation.

Cell Lines and Transfection.

Human embryonic kidney 293 cells (American Type Culture Collection), MDA-MB-435 human breast carcinoma cells, and KS1767 human Kaposi's sarcoma cells (Herndier et al., 1996) were grown in minimal essential media (Irvine Scientific, Santa Ana, Calif.), supplemented with 10% fetal calf serum (FCS) (Tissue Culture Biologicals, Tulare, Calif.). The MO7e leukemia cell line was grown in RPMI 1640 (Irvine Scientific) supplemented with 10% FCS. 5×10⁵ 293 cells were transfected using 5 μg of DNA with the SuperFect transfection reagent (Qiagen) following the manufacturer's recommendations. The incubation time allowed for reporter gene expression was 48 h for dsDNA transfection and 72 h for ssDNA transfection.

Phage Internalization Assay.

MDA-MB435 cells were grown on 8-well chamber plastic slides (Nunc/Nalgene, Houston, Tex.) to 80% confluency. The cells were then incubated with 10¹⁰ phage particles/well for 8 h at 37° C. The cells were washed six times with phosphate-buffered saline (PBS) and treated with glycine buffer (50 mM glycine, pH 2.8; 500 mM NaCl) three times for 10 min to elute externally bound phage particles. The cells were neutralized with PBS and fixed with 4% paraformaldehyde for 15 min at room temperature. Cells were permeabilized with 0.05% saponin (Sigma) in PBS and incubated for 1 h with 1% bovine serum albumin (BSA) in PBS. Subsequent, antibody incubations were performed in 1% BSA/PBS. Cells were incubated with a mouse anti-fd-tet monoclonal antibody at 5 μg/ml. The secondary antibody was Texas Red-conjugated goat anti-mouse polyclonal antibody (Caltag, Burlingame, Calif.). Immunofluorescence detection and analysis were performed on a Nikon CED microscope (Nikon, Dallas, Tex.).

Phage Transduction Assay.

MDA-MB435 and KS1767 cells were grown on 8-well chamber glass slides to 60-80% confluency. The growth medium was replaced by 200 μl MEM containing 2% FCS and 10¹¹ transducing units of either RGD-4Cβ-gal, HWGF-β-gal, or fd-β-gal phage (100,000 phage units/cell). Phage vectors were incubated on cells for 3 h at 37° C., followed by medium change to MEM plus 10% FCS. The cells were incubated for 72 h at 37° C. to allow for β-galactosidase gene expression.

Quantification of Reporter Gene Activity.

In the phage transduction assay, reporter gene expression was analyzed by immunofluorescence. The cells were washed with PBS and permeabilized with 0.2% Triton X-100 for 5 min on ice, followed by -blocking with 1% BSA in PBS. The monoclonal anti-β-galactosidase antibody-(Sigma) diluted 1:2000 in blocking solution was incubated with the cells overnight. A Texas Red-conjugated secondary antibody diluted 1:600 in PBS was incubated with the cells for 1 h. The degree of β-gal expression was determined by counting the fluorescent cells seen under the inverted Nikon microscope.

For the DNA transfections, β-galactosidase enzyme activity was visualized using the In Situ β-galactosidase Staining Kit (Stratagene) following manufacturer's instructions. Cells were incubated with the X-gal substrate for 3 h at 37° C. Blue cells were counted in at least ten fields under the inverted Nikon microscope.

Results

Tumor homing phage were constructed that displayed the RGD4C peptide (Pasqualini et al., 1997) or the HWGT peptide (Koivunen et al., 1999) and contained a β-galactosidase reporter gene under the control of a CMV promoter. These vectors, RGD4C-β-gal and HWGF-β-gal, specifically transduced two tumor cell lines, MDA-MB-435 and KS1767, that express the RGD4C receptor, αvβ3/αvβ5 integrins, or the HWGF receptor, MMP-2/-9, respectively. A control phage with the same transgene cassette but without targeting peptide did not transduce these cell lines. These results are disclosed in Example 8 above.

Transfection of Phage Vector DNA Resulted in Transgene Expression in Mammalian Cells.

To confirm the functionality of the recombinant phage vectors, the double-stranded (ds) phage DNA (replicative form) and the single stranded (ss) phage DNA (infective form) were transfected into 293 human embryonic kidney cells and β-galactosidase transgene expression was determined histochemically. Plasmids with both orientations of the transgene cassette, forward and reverse, were evaluated. While transfection of 293 cells with ds RGD4C-β-gal DNA resulted in β-gal activity in approximately 40% of the cells, transfection with ssDNA produced β-gal activity in 1-3% of the cells (not shown). The orientation of the transgene cassette did not significantly influence the level of β-galactosidase expression (not shown). Therefore, DNA was used with the transgene cassette in the forward orientation in all remaining studies. The transfection data confirmed the functionality of phage-derived hybrid DNA constructs in achieving transgene expression in mammalian cells.

Targeted Phage Vectors are Internalized by Tumor Cells.

An internalization assay was performed to assess whether the targeted phage vectors, RGD4C-β-gal and HGWF-β-gal, could attach to and be internalized into mammalian cells. 10¹⁰ RGD4C-β-gal phage units were added to 5×10⁵ MDA-MB-435 cells and incubated for 8 h at 37° C. KS1767 Kaposi sarcoma cells were used for the incubation with HGWF-β-gal (data not shown). As a control, cells were incubated with the untargeted phage vector, fd-β-gal. Additional controls included cells untreated with phage and cells treated with the targeted phage vector but not permeabilized prior to staining. To ensure that no phage was bound on the cell surface, the cells were washed with glycine buffer.

Immunofluorescence identified phage in the cells that were incubated with the targeted phage vectors, while staining of cells incubated with fd-β-gal was close to the level of background (not shown). Omitting the permeabilization step prior to staining in cells that were incubated with RGD4C-β-gal phage vectors almost completely abolished immunofluorescence, confirming the presence of phage particles exclusively inside the targeted cells (not shown).

Targeted Phage Vectors Transduce Cells Expressing Suitable Receptors.

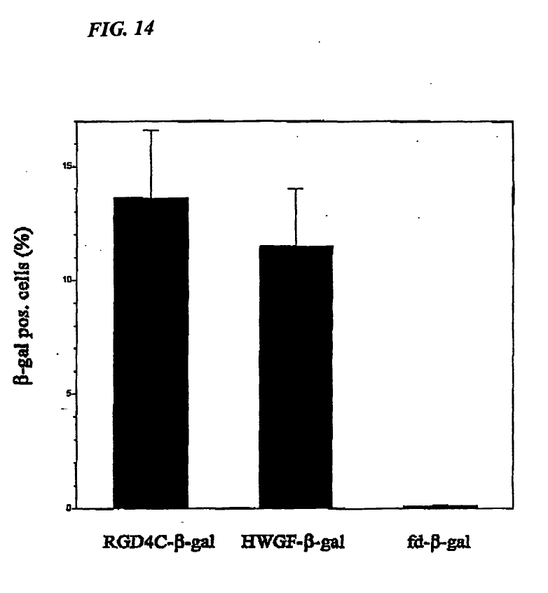

To determine whether RGD-4C-β-gal and HGWF-β-gal could deliver a reporter gene to mammalian cells, those phage vectors were incubated with MDA-MB435 cells, expressing high levels of RGD-4C-receptors (αvβ3 and αvβ5 integrins), or KS1767 cells, expressing high levels of HWGF-receptors (MMP-2 and MMP-9). Anti-β-gal immunofluorescence detected transgene expression in cells treated with the targeted phage vectors, while cells treated with the fd-β-gal vector showed minimal immunofluorescence (FIG. 14). Gene expression was observed in approximately 14% of MDA-MB435 cells after incubation with RGD4C-β-gal phage (FIG. 14) and in approximately 12% of the KS1767 cells incubated with HGWF-β-gal (FIG. 14). β-gal expression in both cell lines treated with fd-β-gal phage resulted in less than 0.1% positive cells (FIG. 14). Control experiments were performed with MO7e cells, which do not express αvβ3 or αvβ5 integrins. Incubation with RGD4C-β-gal or HWGF-β-gal phage only produced minimal β-gal expression (0.31% β-gal positive cells; data not shown). These data suggest that tumor homing phage vectors can be used to achieve significant transgene expression specifically in cells that express the suitable receptors.

Specific uptake of the RGD4C-β-gal phage vector was observed in αvβ3 and αvβ5-positive MDA-MB-435 cells, while MO7e cells showed no β-gal expression. This is consistent with previous reports that RGD displaying phage particles are internalized by integrin-expressing cells (Hart et al., 1994). Although MMP-2 and MMP-9 have been disclosed as targets for cancer therapy, their potential as a receptor for targeted gene delivery has not been studied. The present results show that the HWGF-β-gal phage is internalized and confers β-galactosidase expression in MMP-2 and MMP-9 positive KS1767 cells. The skilled artisan will realize that RGD4C and HWGF-displaying phage may be of use as vectors for targeted gene delivery.

The present example confirms and extends the results of Example 8, demonstrating the feasibility of using targeting peptide modified phage as gene therapy vectors for in vivo or in vitro delivery of therapeutic genes to human cells, tissues and organs.

### Example 10

Targeted Expression of a Model Gene Therapy Vector in Human Cells

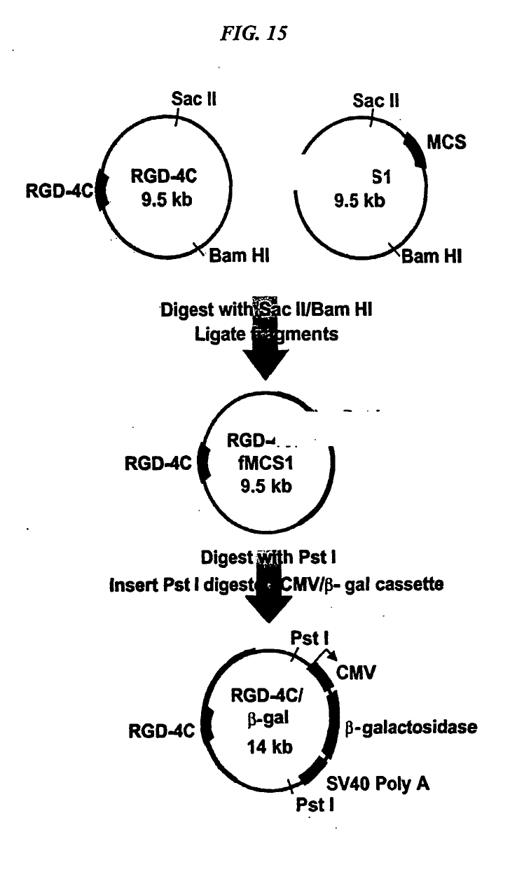

The general protocol for production of human cell targeting phage, described in Examples 8 and 9 above, is illustrated in FIG. 15.

Eukaryotic Cell Expression of a Phage Reporter Gene

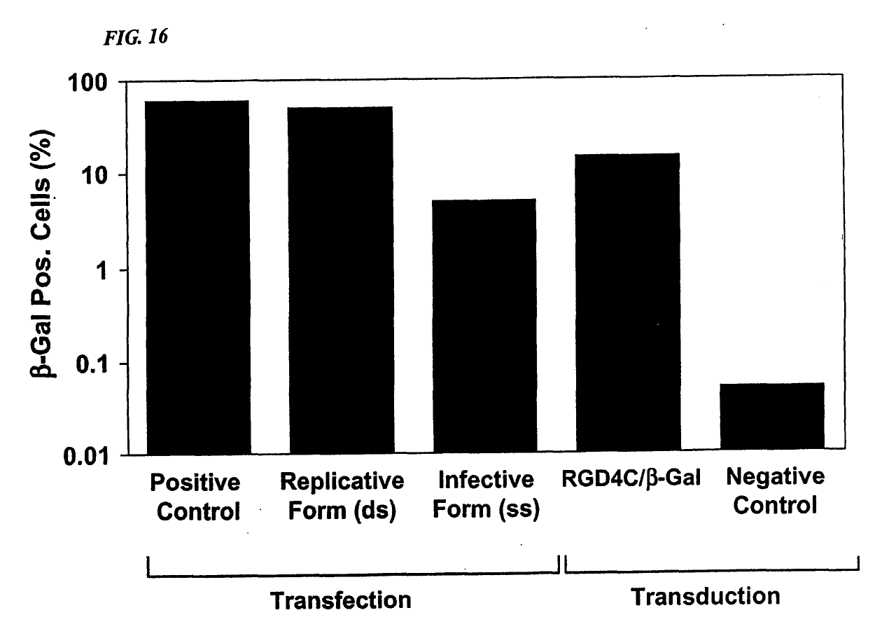

While the replicative form of the phage genome is dsDNA, the infective form of the phage is ssDNA. A LacZ reporter gene embedded in an ss phage genome was tested for expression in human cells. After single-stranded IFs were transferred into human cells, LacZ expression confirmed that the gene was converted from ss DNA to the ds DNA form (FIG. 16).

Phage containing a CMV-βgal cassette were transfected into the human 293 cell line. Both the ds phage genome (RF) and the ss phage genome (IF) were transfected. The extraction of the ss DNA genome from the phage capsid proteins was performed with Strataclean resin (Stratagene), followed by ethanol precipitation. The DNA was quantified by spectrophotometry with 1.0 A₂₆₀ equal to 40 μg/ml for ss DNA or 50 μg/ml for ds DNA. Both ss and ds DNA forms of the phage genome were expressed, as shown by beta-galactosidase activity.

Targeted Phage Undergo Receptor-Mediated Internalization

A phage internalization assay was used to show that RGD4C-βGal exhibits receptor-mediated cell uptake in human cancer cells. Human KRIB sarcoma cells or angiogenic KS1767 Kaposi sarcoma cells were used to determine whether RGD4C-βgal phage can deliver a reporter gene to mammalian cells for expression. After cells were grown to 70% confluency, RGD4C-βgal phage or a control phage with no insert were added at 10¹⁰ T.U./well and cells were incubated for 8 hr at 37° C. Cells were washed 6 times with PBS and treated with glycine buffer 3 times to elute externally bound phage. Cells were fixed with 4% paraformaldehyde for 15 minutes at RT, permeabilized with 0.05% saponin/TBS or treagted with TBS alone, stained with an anti-M13 antibody, and counterstained with hematoxylin. Cells were treated with RGD4C-βgal phage with or without permeabilization or were treated with control phage with no insert with or without permeabilization.

The results showed that RGD4C-βgal phage were internalized in the absence of permeabilization, while control phage were not (data not shown). Significant βgal gene expression was observed with a multiplicity of infection (MOI) as low as 10 and a dose-response was observed between MOIs of 10 to 10⁵ (data not shown). Strong LacZ staining was seen in about 15-20% of the cells after incubation with the RGD4C-βgal phage. The endogenous background activity observed in the negative control cells was more than two orders of magnitude less than the RGD4C-βgal phage treated cells. In a separate study, co-administration of soluble RGD4C peptide was shown to inhibit the interaction of RGD4C phage with its cell surface receptor (Arap et al., 1998b; Pasqualini et al., 1997).

Intravenous protein administration is possible and appears to be safe in the case of angiogenesis inhibitors such as angiostatin and endostatin. However, these inhibitors must be injected at very high doses on a weekly basis to produce long-lasting anti-tumor effects. Therapy cannot be interrupted without the recurrence of the tumor, unless the proteins are given over a period of many months (Boehm et al., 1997). Genetic therapy based on the inhibition of angiogenesis appears to be feasible since, in the case of endostatin, the protein has been shown to be biologically active when secreted from gene-transduced cells (Cao et al., 1998; Griscelli et al., 1998; Tanaka et al., 1998). However, vector systems suitable for this kind of treatment would have to promote high and long-lasting expression.

The AAP vectors described in Example 8 may have advantages for anti-angiogenic gene therapy because they are known to generate high, stable levels of gene expression. Recent data from a number of groups indicates that AAP-type vectors may be particularly useful in skeletal muscle, where reporter expression has been demonstrated over one year following a single injection (Muzyczka et al., 1994).

In vivo screenings were performed to isolate a panel of muscle-homing phage. When injected intravenously, the phage accumulated specifically within the vasculature, and at later time points, in muscle tissue. These results may be of use for targeted AAP transduction of skeletal muscle as a depot for sustained secretion of endostatin. AAP vectors combined with muscle homing peptides may be used to increase the level and duration of gene expression in skeletal muscle.

### Example 11

Screening Phage Libraries by PALM

In certain embodiments, it is desirable to be able to select specific cell types from a heterogeneous sample of an organ or tissue. One method to accomplish such selective sampling is by PALM (Positioning and Ablation with Laser Microbeams).

The PALM Robot-MicroBeam uses a precise, computer-guided laser for microablation. A pulsed ultra-violet (UV) laser is interfaced into a microscope and focused through an objective to a beam spot size of less than 1 micrometer in diameter. The principle of laser cutting is a locally restricted ablative photodecomposition process without heating (Hendrix, 1999). The effective laser energy is concentrated on the minute focal spot only and most biological objects are transparent for the applied laser wavelength. This system appears to be the tool of choice for recovery of homogeneous cell populations or even single cells or subcellular structures for subsequent phage recovery. Tissue samples may be retrieved by circumcising a selected zone or a single cell after phage administration to the subject. A clear-cut gap between selected and non-selected area is typically obtained. The isolated tissue specimen can be ejected from the object plane and catapulted directly into the cap of a common microfuge tube in an entirely non-contact manner. The basics of this so called Laser Pressure Catapulting (LPC) method is believed to be the laser pressure force that develops under the specimen, caused by the extremely high photon density of the precisely focused laser microbeam. This tissue harvesting technique allows the phage to survive the microdissection procedure and be rescued.

PALM was used in the present example to select targeting phage for mouse pancreatic tissue, as described below.

Materials and Methods

In vitro Panning

A CX₇C peptide phage library (10⁹ TU) was pre-screened by injected into the tail vein of a C57BL/6 male mouse, and the pancreas was harvested to recover the phage by bacterial infection. Phage from 246 colonies were grown separately in 5 mls LB/kanamycin (100 μg/ml)/tetracycline (40 μg/ml) at 37° C. in the dark with agitation. Overnight cultures were pooled and the phage purified by NaCl/PEG precipitation for another round of in vivo bio-panning. Three hundred colonies were picked from the second round of panning, and the phage were recovered by precipitation. Phage from the second bio-panning round was then used for another round of in vivo panning and also was incubated with thawed frozen murine pancreatic sections for one in vitro panning round. For the third in vivo panning round, 10⁹ TU phage from the second round were injected into a third mouse and allowed to circulate for six minutes, followed by an intravenous injection of 50 μl of FITC-lectin (Vector Laboratories, Inc.). After a two-minute circulation, the mouse was perfused through the left ventricle with 3 mls MEM Earle salts. The pancreas was harvested, frozen at ⁻80 ° C. in Tissue Tek (Sakura), and sectioned onto prepared slides.

For the third in vitro round, purified phage, isolated from the second round, were incubated with 4-14 μm thawed murine pancreatic sections on ice for 30 minutes. Sections were rinsed with 100 μl ice-cold PBS 8× at room temperature (RT). Bound phage were recovered from each section by adding 100 μl K91 Kan^{R} (OD₆₀₀=2.03) to infect at RT for 30-60 minutes. Infected K91 KanR were withdrawn from each section and allowed to recover in 10 mls LB/Kan/Tet (0.2 μg/ml) for 20 minutes in the dark. Aliquots from the each culture were plated out onto LB/Kan/Tet (40 μg/ml) plates and incubated overnight in the dark at 37° C. The tetracycline concentration of the remainder of each culture was increased to 40 μg/ml and the cultures were incubated overnight at 37° C. in the dark with agitation for phage amplification and purification.

DNA Amplification

Phage were recovered from cryo-preserved FITC-lectin stained mouse pancreatic islets and surrounding acinar cells that were microdissected from 14 μm sections using the PALM (Positioning and Ablation with Laser Microbeams) cold laser pressure catapulting system. Pancreatic islet and control sections were catapulted into 1 mM EDTA, pH 8, and frozen at −20° C. until enough material was collected for PCR amplification. Phage DNA was amplified with fUSE5 primers: forward primer 5′ TAA TAC GAC TCA CTA TAG GGC AAG CTG ATA AAC CGA TAC AATT 3′ (SEQ ID NO: 170), reverse primer 5′ CCC TCA TAG TTA GCG TAA CGA TCT 3′ (SEQ ID NO: 171). The PCR products were subjected to another round of PCR using a nested set of primers. The 3′ end of the second primer set was tailed with the M13 reverse primer for sequencing purposes. The nested primer set used was: forward nested primer 5′ CCTTTCTATTCTCACTCGGCCG 3′ (SEQ ID NO: 172), reverse nested primer 5′ CAGGAAACAGCTATGACCGCTAAACAACTTTCAACAGTTTCGGC 3′ (SEQ ID NO: 173). To generate peptide insert sequence containing flanking SfiI restriction sites, two more primers were used: forward library primer 5′ CACTCGGCCGACGGGGC 3′ (SEQ ID NO: 174), reverse primer 5′ CAGTTTCGGCCCCAGCGGCCC 3′ (SEQ ID NO: 175). PCR products generated from the nested primers were gel purified (Qiagen), and confirmed for the presence of a CX₇C peptide insert sequence using the M13 reverse primer by automated sequencing. PCR products generated from the library primers were gel purified (Qiagen), ligated into CsCl₂ purified fUSE5/SfiI, electroporated into electrocompetent MC1061 cells, and plated onto LB/streptomycin (100 μg/ml)/tetracycline (40 μg/ml) agar plates. Single colonies were subjected to colony PCR using the fUSE5 primers to verify the presence of a CX₇C insert sequence by gel electrophoresis. Positive clones were sequenced using BigDye terminators (Perkin Elmer)

Phage Infection

Pancreatic islet and control sections were catapulted into 1 mM AEBSF, 20 μg/ml aprotinin, 10 μg/ml leupeptin, 1 mM elastase inhibitor I, 0.1 mM TPCK, 1 nM pepstatin A in PBS, pH 7.4, and frozen for 48 hours or less until enough material was collected. The sections were thawed on ice and the volume adjusted to 200 μl with PBS, pH 7.4. Samples were incubated with 1 ml K91 Kan^{R} (OD=0.22) for two hours at RT on a nutator. Each culture was transferred to 1.2 mls LB/Kan/Tet (0.2 μg/ml) and incubated in the dark at RT for 40 minutes. The tetracycline concentration was increased to 40 μg/ml for each culture, and the cultures were incubated overnight at 37° C. with agitation. Each culture was plated out the following day onto LB/Kan/Tet agar plates and incubated for 14 hours at 37° C. in the dark. Positive clones were picked for colony PCR and automated sequencing.

Results

After an initial round of in vivo selection, phage were either bulk amplified or else single colonies of phage from pancreas, kidney, lung and adrenal glands were amplified and subjected to additional rounds of in vivo screening. Both bulk amplified and colony amplified phage from mouse pancreas showed successive enrichment with increasing rounds of selection (not shown). After three rounds of selection, the colony amplified phage showed almost an order of magnitude higher enrichment than bulk amplified phage (not shown).

Table 12 lists selected targeting sequences and consensus motifs identified by pancreatic screening.

TABLE 12
Pancreatic targeting peptides and motifs

| Motif | Peptide Sequence | |
|---|---|---|
| GGL | CVPGLGGLC | (SEQ ID NO:193) |
| (SEQ ID NO:176) | | |
| | CGGLDVRMC | (SEQ ID NO:194) |
| | CDGGLDWYC | (SEQ ID NO:195) |
| LGG | CVPGLGGLC | (SEQ ID NO:193) |
| (SEQ ID NO:177) | | |
| | CTWLGGREC | (SEQ ID NO:196) |
| | CSRWGLGGC | (SEQ ID NO:197) |
| | CPPLGGSRC | (SEQ ID NO:198) |
| VRG | CVGGVRGGC | (SEQ ID NO:199) |
| (SEQ ID NO:178) | | |
| | CVGNDVRGC | (SEQ ID NO:200) |
| | CESRLVRGC | (SEQ ID NO:201) |
| | CGGRPVRGC | (SEQ ID NO:202) |
| AGG | CTPFIAGGC | (SEQ ID NO:203) |
| (SEQ ID NO:179) | | |
| | CREWMAGGC | (SEQ ID NO:204) |
| | CAGGSLRVC | (SEQ ID NO:205) |
| VVG | CEGVVGIVC | (SEQ ID NO:206) |
| (SEQ ID NO:180) | | |
| | CDSVVGAWC | (SEQ ID NO:207) |
| | CRTAVVGSC | (SEQ ID NO:208) |
| VGG | CVGGARALC | (SEQ ID NO:209) |
| (SEQ ID NO:181) | | |
| | CVGGVRGGC | (SEQ ID NO:199) |
| | CLAHRVGGC | (SEQ ID NO:210) |
| GGL | CWALSGGLC | (SEQ ID NO:211) |
| (SEQ ID NO:182) | | |
| | CGGLVAYGC | (SEQ ID NO:212) |
| | CGGLATTTC | (SEQ ID NO:213) |
| GRV | CGRVNSVAC | (SEQ ID NO:214) |
| (SEQ ID NO:183) | | |
| | CAGRVALRC | (SEQ ID NO:215) |
| GGA | CWNGGARAC | (SEQ ID NO:216) |
| (SEQ ID NO:184) | | |
| | CLDRGGAHC | (SEQ ID NO:217) |
| GVV | CELRGVVVC | (SEQ ID NO:218) |
| (SEQ ID NO:185) | | |
| GGV | CIGGVHYAC | (SEQ ID NO:219) |
| (SEQ ID NO:186) | | |
| | CGGVHALRC | (SEQ ID NO:220) |
| GMWG | CIREGMWGC | (SEQ ID NO:221) |
| (SEQ ID NO:187) | | |
| | CIRKGMWGC | (SEQ ID NO:222) |
| ALR | CGGVHALRC | (SEQ ID NO:220) |
| (SEQ ID NO:188) | | |
| | CAGRVALRC | (SEQ ID NO:215) |
| | CEALRLRAC | (SEQ ID NO:223) |
| ALV | CALVNVHLC | (SEQ ID NO:224) |
| (SEQ ID NO:189) | | |
| | CALVMVGAC | (SEQ ID NO:225) |
| GGVH | CGGVHALRC | (SEQ ID NO:220) |
| (SEQ ID NO:190) | | |
| | CIGGVHYAC | (SEQ ID NO:219) |
| VSG | CMVSGVLLC | (SEQ ID NO:226) |
| (SEQ ID NO:191) | | |
| | CGLVSGPWC | (SEQ ID NO:227) |
| | CLYDVSGGC | (SEQ ID NO:228) |
| GPW | CSKVGPWWC | (SEQ ID NO:229) |
| (SEQ ID NO:192) | | |
| | CGLVSGPWC | (SEQ ID NO:230) |
| none | CAHHALMEC | (SEQ ID NO:231) |
| | CERPPFLDC | (SEQ ID NO:232) |

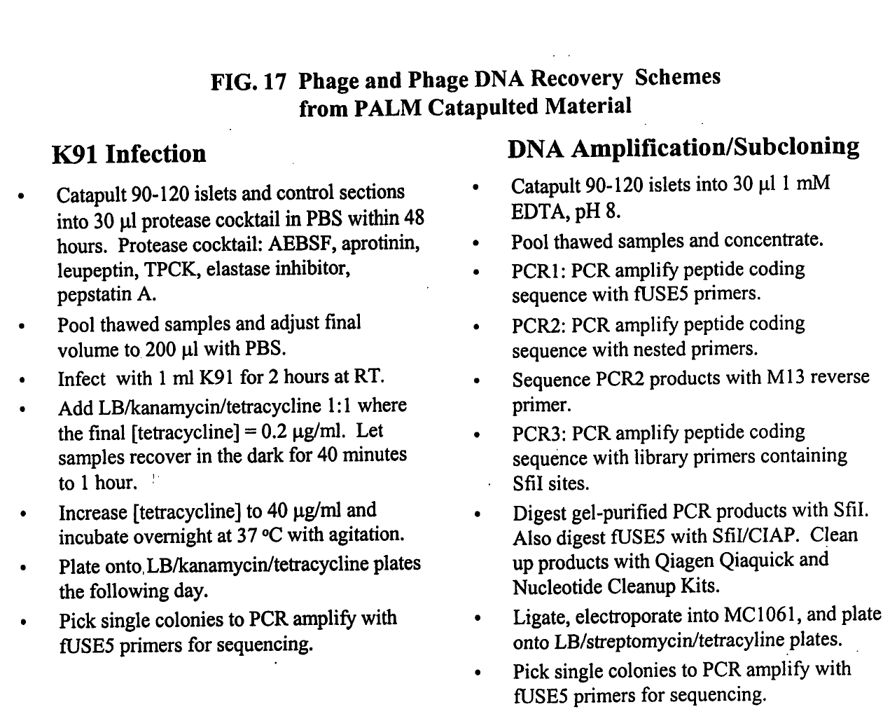

FIG. 17 shows a general protocol for recovery of phage insert sequences from PALM selected thin section materials. As indicated, phage may be recovered by direct infection of E. coli host bacteria, after protease digestion of the thin section sample. Alternatively, phage inserts may be recovered by PCR amplification and cloned into new vector DNA, then electroporated or otherwise transformed into host bacteria for cloning.

Both methods of PALM recovery of phage were successful in retrieving pancreatic targeting sequences. Pancreatic sequences recovered by direct bacterial infection included CVPRRWDVC (SEQ ID NO: 233), CQHTSGRGC (SEQ ID NO: 234), CRARGWLLC (SEQ ID NO: 235), CVSNPRWKC (SEQ ID NO: 236), CGGVHALRC (SEQ ID NO: 220), CFNRTWIGC (SEQ ID NO: 237) and CSRGPAWGC (SEQ ID NO: 238). Pancreatic targeting sequences recovered by amplification of phage inserts and cloning into phage include CWSRGQGGC (SEQ ID NO: 239), CHVLWSTRC (SEQ ID NO: 240), CLGLLMAGC (SEQ ID NO: 241), CMSSPGVAC (SEQ ID NO: 242), CLASGMDAC (SEQ ID NO: 243), CHDERTGRC (SEQ ID NO: 244), CAHHALMEC (SEQ ID NO: 245), CMQGAATSC (SEQ ID NO: 246), CMQGARTSC (SEQ ID NO: 247) and CVRDLLTGC (SEQ ID NO: 248).

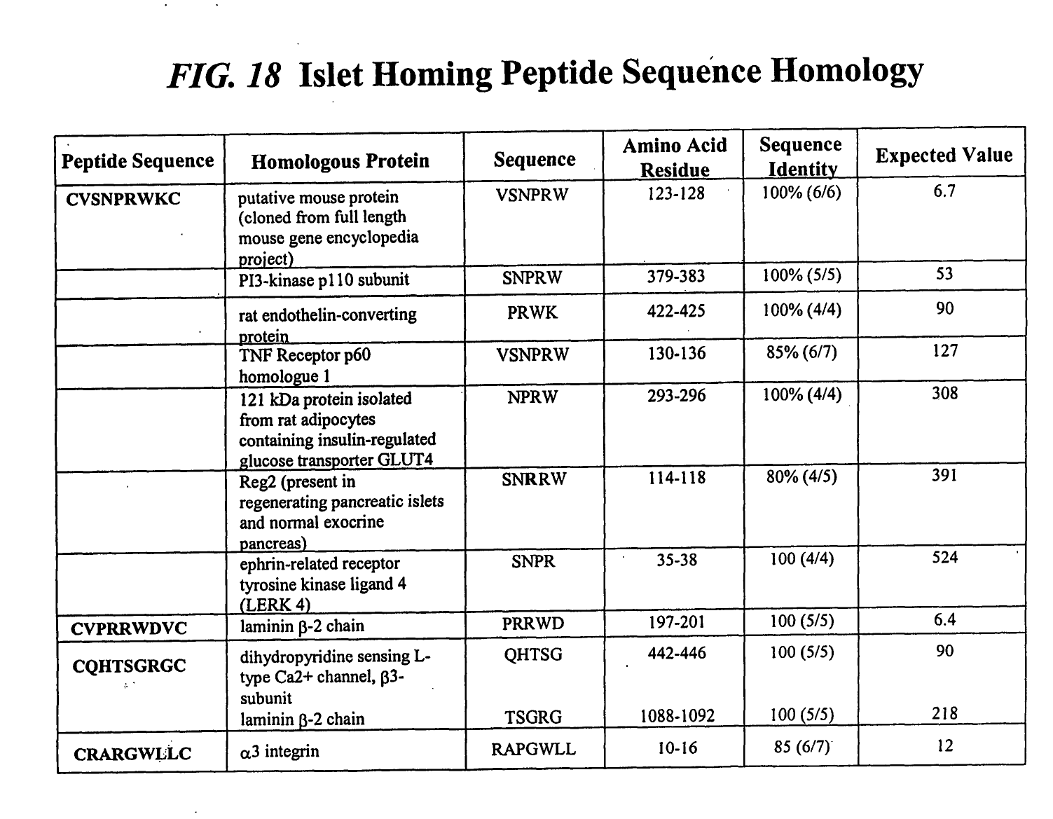

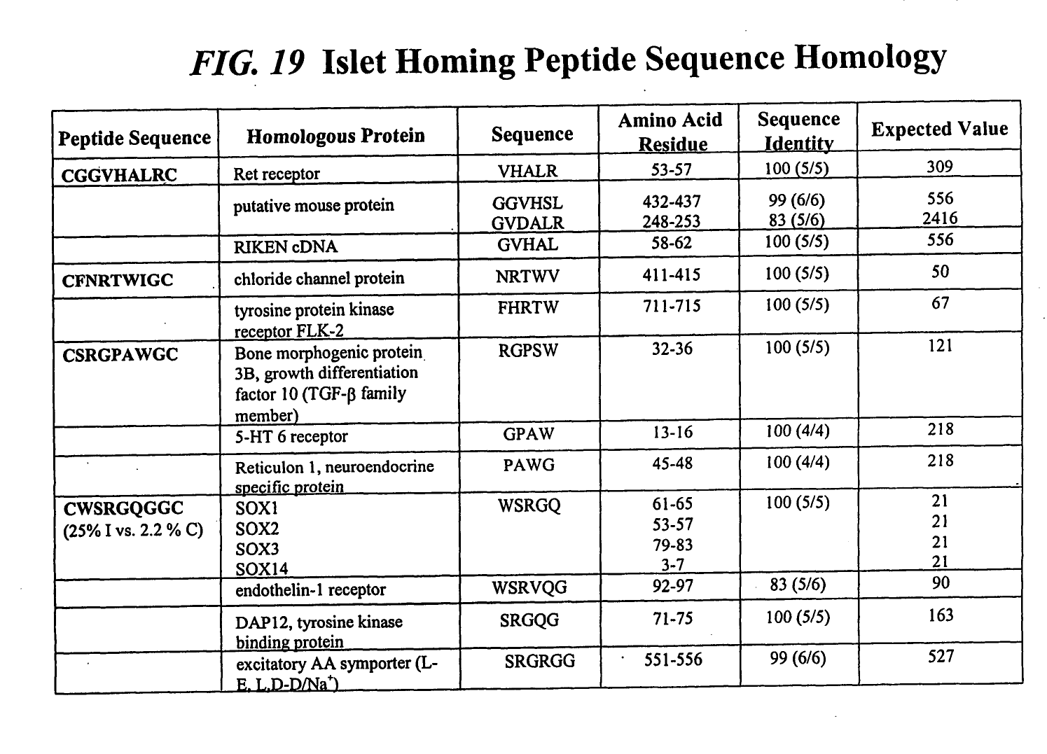

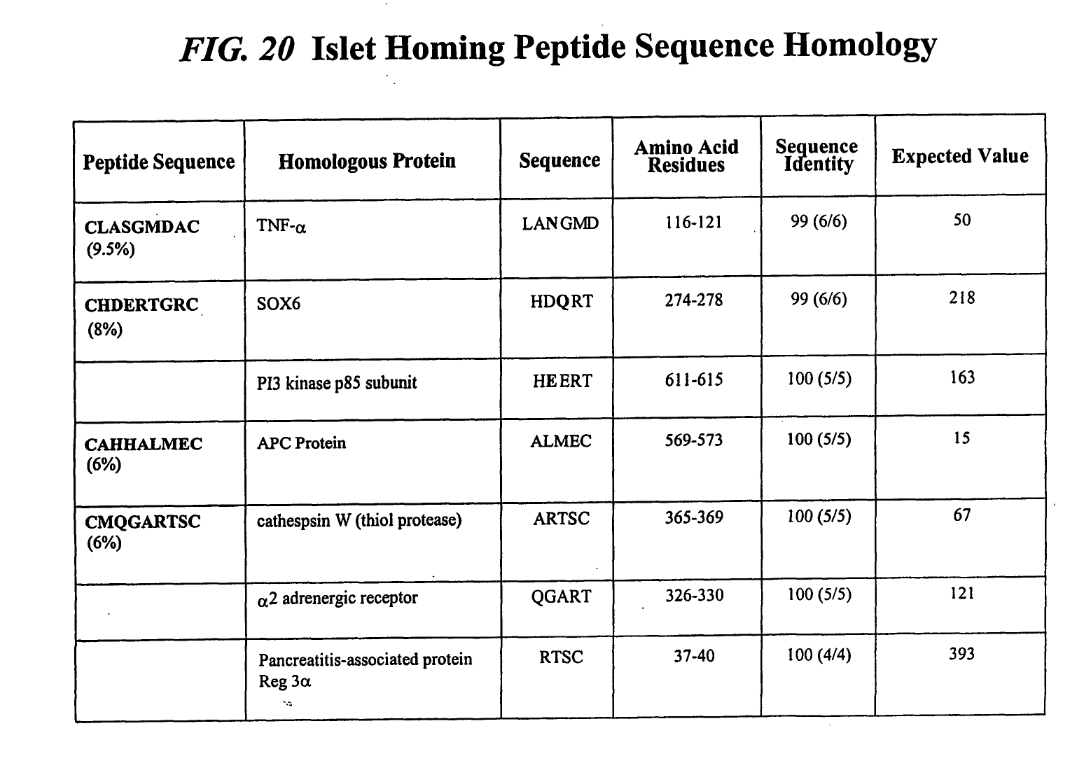

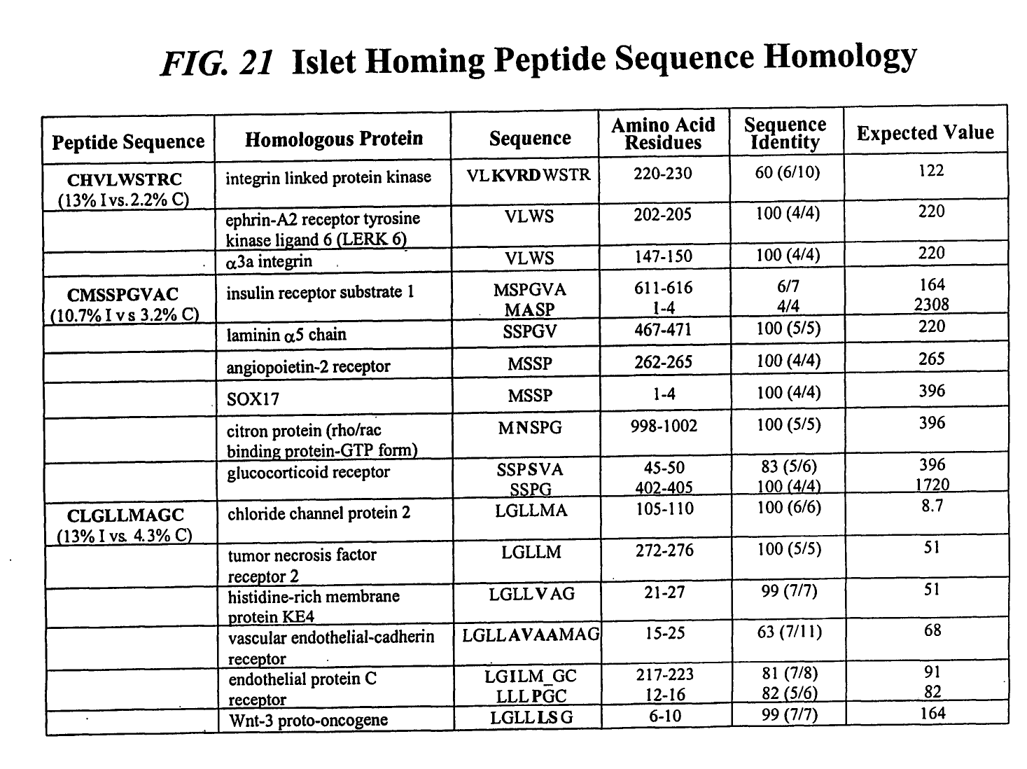

FIG. 18 through FIG. 21 show sequence homologies identified for selected pancreatic targeting sequences. Several proteins known to be present in pancreatic tissues were identified. The results of this example show that the PALM method may be used for selecting cell types from tissue thin sections and recovering targeting phage sequences. The skilled artisan will realize that this method could be used with virtually any tissue to obtain targeting sequences directed to specific types of cells in heterologous organs, tissues or cell types.

### Example 12

Novel Markers for Ovarian Cancer Identified from Ascites Fluid Screened by Phage Display

Ovarian cancer is the fifth most common cancer among American women, with 23,000 new cases diagnosed annually. The five-year survival rates for ovarian cancer by stage are: Stage I (93%), Stage II (70%), Stage III (37%), and Stage IV (25%). As with all cancers, delayed detection results in a drastic reduction in survival rate. Approximately two thirds of patients are currently diagnosed with advanced stage disease. Most patients are asymptomatic or have only vague symptoms such as abdominal or pelvic fullness before metastasis occurs. The majority of women who have been successfully treated for ovarian cancer and in whom tumor control is achieved will eventually develop recurrent disease. More women die of ovarian cancer than from all other gynecologic malignancies combined.

Although a number of tumor markers in ovarian cancer have been identified, no marker as of yet has a clear role in the diagnosis and/or prognosis of ovarian cancer. Of the known markers, CA125 is the most extensively studied. It has a well-defined and validated role as a reliable indicator of response or progression. However, it is a poor predictor of long term prognosis (Maggino and Gadduci, 2000; Mayer and Rustin, 2000).

Ovarian malignancy may result in the accumulation of ascitic fluid in the peritoneal cavity. This exudate often contains tumor cells as well as tumor-related compounds such as carcinoembryonic antigen (Booth et al., 1977; Breborowicz et al., 1977), α fetoproteins (Khoo and Mackay, 1977), glycoproteins (Booth et al., 1977) and many tumor associated immunoglobulins (Dorsett et al, 1975). It has also been shown that immunoglobulins isolated from ascitic fluid react with the serum as well as tumor tissue from the patient from which the ascitic fluid was isolated (Hill et al., 1978).

The present example shows that in vitro phage display may be used to screen immunoglobulins from the ascitic fluid of ovarian cancer patients and identify markers for the disease. These results represent a significant advance concerning markers for ovarian cancer diagnosis and and targeting peptides for ovarian cancer treatment. The clinical applications of this work range from the development of vaccines and/or anti-idiotype antibodies for immunotherapy, increased accuracy for diagnostic/prognostic, testing, and directed specificity in tumor targeting

Materials and Methods

Ascitic Samples

Ascites was collected into sterile containers and centrifuged at 2500 rpm for 20 min to separate the cell free ascitic fluid from the cellular fraction. The fluid was stored at −20° C. and the cellular fraction washed twice in phosphate-buffered saline (PBS). Erythrocytes were lysed using 0.17 M Tris-HCl, 0.16 M NH₄Cl, pH 8.3, while shaking at 37° C. for 10 minutes. The remaining cellular fraction was washed one more time in PBS and then maintained in monolayer culture in growth medium consisting of RPMI 1640 and DMEM with 5% fetal bovine serum. In cultures in which there appear to be co-contamination with normal fibroblasts, a technique resulting in differential, attachment to plastic by means of sequential reductions in serum concentration was used to purify the ovarian cancer tumor cells.

Phage Display/Biopannings

To select peptides specific to the ascitic fluid of ovarian cancer patients, IgGs from normal donor serum and ovarian cancer cell free ascitic fluid were bound to Protein agarose (Pierce) in the Pierce acetate, pH5 binding buffer. A two step biopanning procedure was employed in which the first step involved pre-clearing the phage peptide library by incubating it with IgGs isolated from normal donor serum in order to remove common antigens. This was followed by a second step where the pre-cleared library was used to screen cancer specific IgGs. Approximately 10⁸ transforming units were added to the IgGs for the panning procedure. The resultant IgG-bound phage were recovered by eluting the phage with 0.1M glycine buffer, pH2.2, neutralizing the phage with 0.1 volume 1 M Tris-Cl, pH 9, and using the phage to infect stationary phase Escherichia coli strain K91. Serial dilutions of phage infected-K91 were plated onto tetracycline (40 μg/ml) LB agar plates and grown overnight at 37° C. Individual clones were picked, amplified, and precipitated for subsequent rounds of panning. A total of three rounds of selection were performed. Phage clones from the second and third rounds were subject to PCR followed by sequence analysis to evaluate enrichment of the most consistently binding peptide sequences.

Peptide specific sequences to ovarian cancer cells may also be selected by utilizing BRASIL. BRASIL is based on the fast separation of phage bound to cells from an aqueous medium into an oil phase. Cells mixed with a phage display library are layered on an oil phase and centrifuged. Because intact cells are denser than the oil, they pellet at the bottom of the tube. Only the phage bound to the cell surface can pass through the oil. Unbound, water-soluble phage are left in the aqueous phase at the top of the tube. Ovarian cancer cells isolated from ascitic fluid may be harvested with PBS and 1% EDTA (5 minutes), washed with PBS, resuspended in MEM containing 1% BSA at 10⁶ cells/ml and incubated with phage on ice. After 4 h, 100 μl of the cell suspension is transferred to a 400 μl Eppendorf tube containing 200 μl of a dibutyl phthalate:cyclohexane mixture (9:1) and centrifuged at 10.000 g for 10 minutes. The tubes are snap frozen in liquid N₂, the bottom of the tubes were cut off, and the pellets transferred to a new tube. The phage bound to cancer cells are rescued by infection with 200 μl of Escherichia coli strain K91kan cells in log phase. Preferably, three rounds of selection are performed. Subcloning, Expression, and Purification of GST fusion Proteins

Peptide coding sequences of interest obtained from selection were amplified by colony PCR and cloned into the GST vector pGEX-2TK (Amersham/Pharmacia) at the BamHI-EcoRI sites. Automated sequencing was used for verification of positive clones. Positive clones were transformed into the bacterial expression host strain, BL21 (DE3) pLys (Strategene), by electroporation. GST fusion proteins were affinity purified from bacterial lysates by affinity chromotography using glutathione Sepharose 4B resin (Amersham/Pharmacia) in 0.02 M Tris-Cl, pH 8.0, 0.1 M NaCl, 1 mM EDTA, 20% NP40.

Enzyme-Linked Immunosorbent Assay (ELISA)

Affinity purified GST fusion proteins were used to screen banked ascitic fluid and serum from ovarian cancer patients by ELISA. A solution of either GST or GST fusion proteins in 0.1 M NaHCO₃ were used to coat maxisorp multi-well plates (Nalge Nunc International Corporation) at 1 μg/well at 4° C. overnight. Following coating, the plates were rinsed and subsequently blocked with a blocking buffer composed of 4% milk, 2% casein, and 0.05% Tween-20 for approximately 3-4 hours. Ascitic fluid or serum was applied to the coated and blocked wells at varying dilutions. After allowing the ascitic fluid or serum to interact with the fusion proteins, the plates were washed with a washing buffer (1% milk, 0.5% casein, and .025% Tween-20) and anti-human alkaline phosphatase (Sigma) was added to each well. The colorimetric signal was developed using p-nitrophenyl phosphate (Sigma) and measuring OD₄₀₅. Background signals were determined with GST alone and normal donor serum.

Biotinylation and Purification of IgG Antibodies

GST fusion proteins made from inserting recombinant peptide sequences of interest in an expression vector were coated on maxisorp multi-well plates (Nalge Nunc International Corporation). The plates are incubated with the ascitic fluid from which the peptide was originally isolated. Following a washing procedure to remove unbound IgGs, bound IgGs were eluted with 0.1 M glycine buffer, pH2.2, neutralized with 1 M Tris-Cl, pH9.0, and dialyzed in PBS overnight. To concentrate the IgG, centricon-30 columns (Millipore) were used.

Results

The present example demonstrates that circulating antibodies against ovarian cancer cell surface markers are present in ascites fluid samples. Peptides that are mimeotopes of the endogenous cancer markers may be identified by phage display panning against ascites immunoglobulins. Peptide motifs identified by panning ascitic IgG (immunoglobulin gamma) are listed in Table 13.

TABLE 13
Ovarian cancer targeting peptide motifs identified
by ascites screening

| | |
|---|---|
| VPELGHE | (SEQ ID NO:249) |
| ELGFELG | (SEQ ID NO:250) |
| MGDTGHC | (SEQ ID NO:251) |
| LEFNLGY | (SEQ ID NO:252) |
| FFLRDWF | (SEQ ID NO:253) |
| YRLRG | (SEQ ID NO:254) |
| YRARG | (SEQ ID NO:255) |
| SQPLG | (SEQ ID NO:256) |
| SQPWG | (SEQ ID NO:257) |
| QRLVTP | (SEQ ID NO:258) |
| QVLVTP | (SEQ ID NO:259) |
| QRLVHP | (SEQ ID NO:260 |
| QVLVHP | (SEQ ID NO:261) |
| ITRWRYL | (SEQ ID NO:262) |
| SLGGMSG | (SEQ ID NO:263) |
| SQLAAG | (SEQ ID NO:264) |
| SQLVAG | (SEQ ID NO:265) |
| SLLAAG | (SEQ ID NO:266) |
| SLLVAG | (SEQ ID NO:267) |
| GLPSGL | (SEQ ID NO:268) |
| HGGSANP | (SEQ ID NO:269) |
| SLEAFFL | (SEQ ID NO 270) |

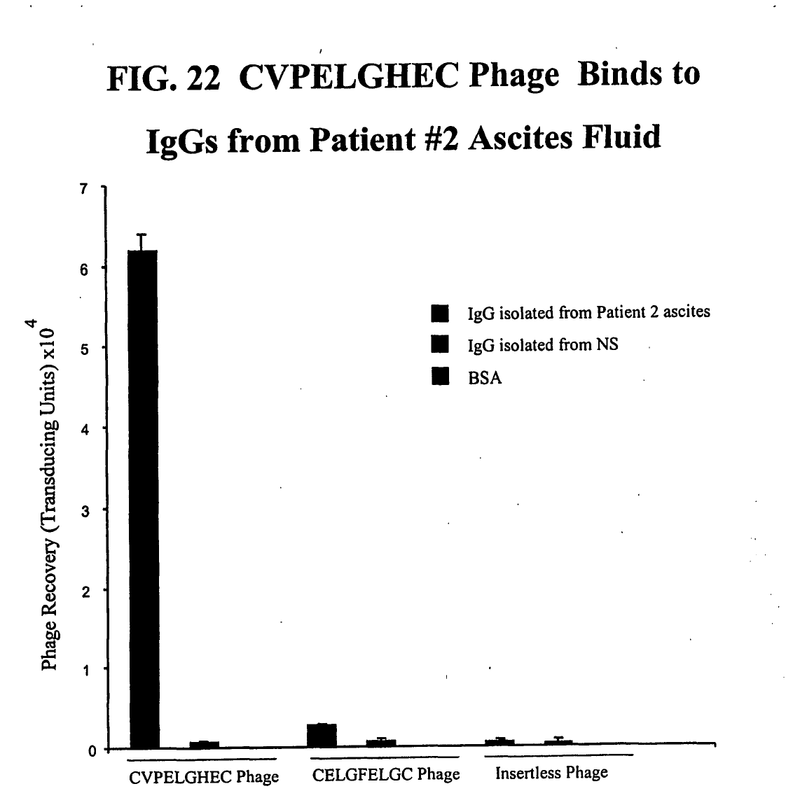

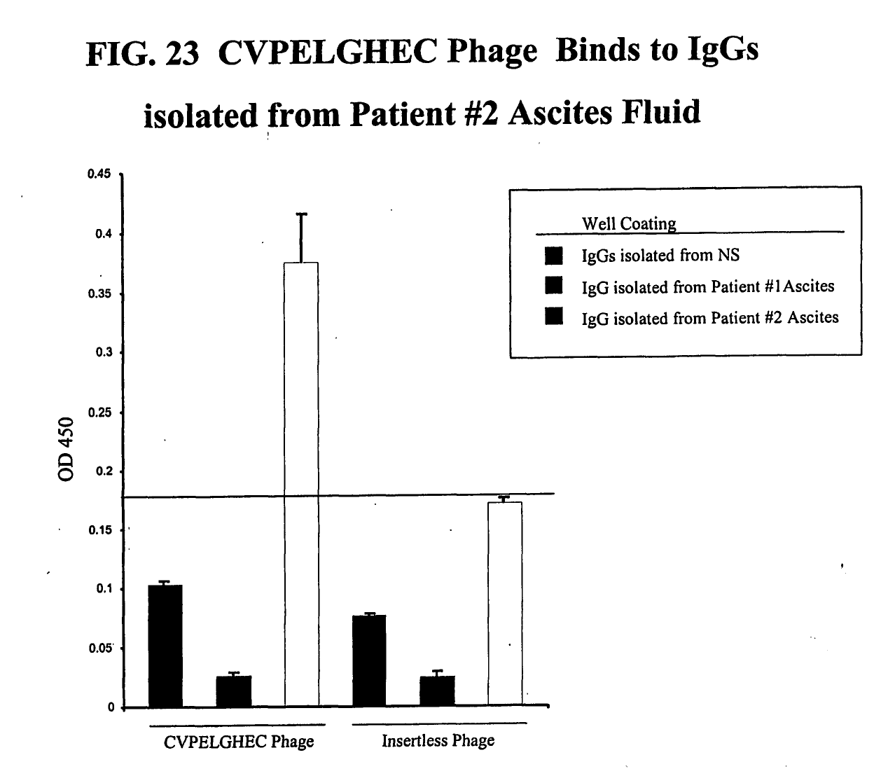

Ascites fluid from several ovarian cancer patients was screened against a phase display library. After three rounds of panning, phage were selected that bound with relatively high affinity to ascites fluid. The targeting sequences CVPELGHEC (SEQ ID NO: 271) and CFELGFELGC (SEQ ID NO: 272) were selected for further studies. FIG. 22 shows that phage bearing the sequence CVPELGHEC (SEQ ID NO: 271) bind with very high selectivity to IgG from the ovarian cancer patient, but not to IgG from a normal patient or to BSA. The phage binding was also selective for ascites from the ovarian cancer patient from which the phage sequence was originally selected (patient #2). The CVPELGHEC (SEQ ID NO: 271) phage bound with much higher affinity to IgGs from patient #2, compared to patient #1 or to normal individuals (FIG. 23).

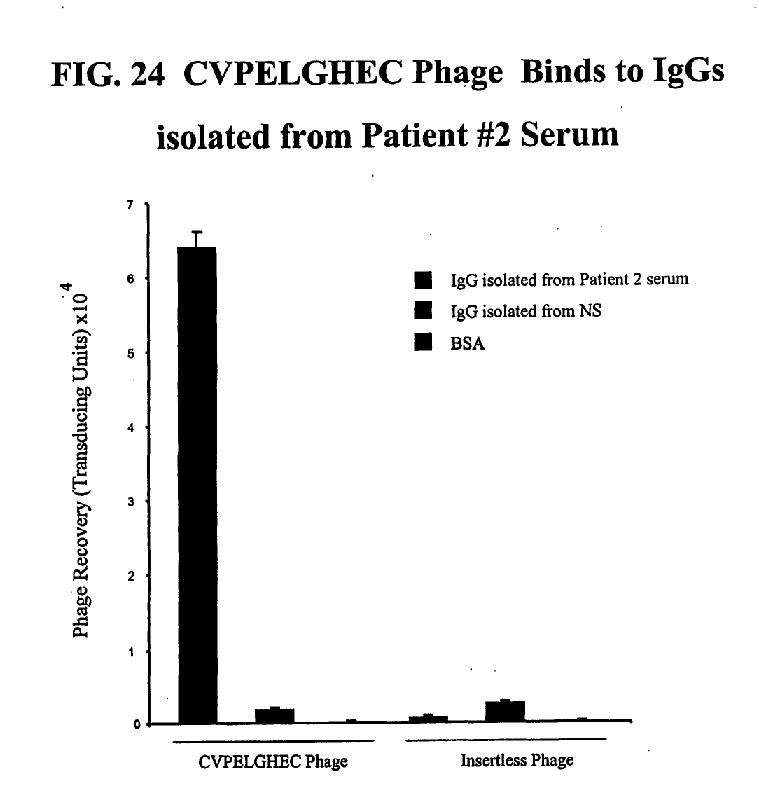

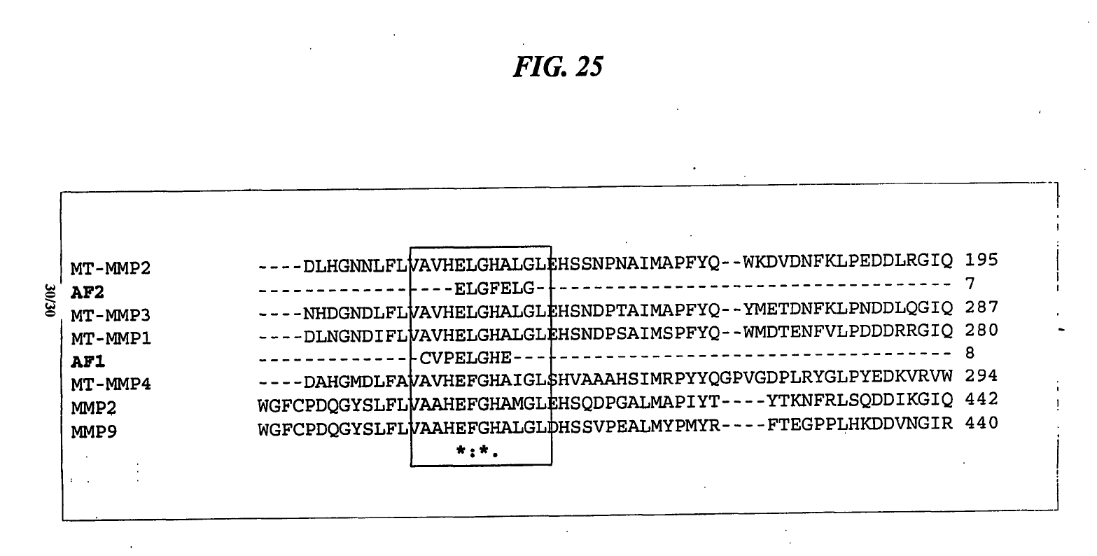

The circulating antibody that the CVPELGHEC (SEQ ID NO: 271) sequence bound to was present in the blood serum of patient #2. The phage bound with similarly high selectivity to serum from patient #2, compared to BSA or normal serum (FIG. 24). Peptide motifs identified by panning against patient #2 ascites showed homology to the catalytic domain of matrix metalloproteinases (MMPs), as shown in FIG. 25.

Homology searches identified several other candidate protein homologs for the ovarian cancer targeting peptides or motifs identified by ascites screening, including an unnamed protein product from HUVEC cells (ITRWRYL, SEQ ID NO: 267); coxsackie and adenovirus receptor protein (SLGGMSG, SEQ ID NO: 263); estrogen receptor (GLPSGL, SEQ ID NO: 268), TSH, FSH, LH and β-hCG receptor (CVPELGHEC, SEQ ID NO: 271, CELGFELGC, SEQ ID NO: 272); endothelin-converting enzyme (ELGFELG, SEQ ID NO: 250), and fibronectin leucine rich transmembrane protein 1 (FFLRDWF, SEQ ID NO: 253).

These results show that ascites fluid and serum from ovarian cancer patients contain immunoglobulins that may be used for screening phage display libraries for mimeotopes of ovarian cancer marker proteins. The targeting peptides identified herein may be used to purify antibodies against ovarian cancer markers or as antigens to produce monoclonal or polyclonal antibodies against ovarian cancer markers. The peptides and antibodies are also of use for identifying the endogenous ovarian cancer antigenic proteins against which the circulating IgG's are induced. Such antibodies may be of use for ovarian cancer diagnosis and/or prognosis, for imaging ovarian cancer, for anti-cancer therapy and for targeted delivery of anti-cancer agents. The skilled artisan will realize that althought the present example deals with ovarian cancer, the methods disclosed may be applied to markers for any type of cancer or any other disease state against which circulating antibodies may be found in blood, ascites, lymphatic fluid or any other sample from an individual suspected of exhibiting cancer or another disease.

### Example 13

Identification of Targets in Tissue Sections: CD₁₃ in Tumors

Material and Methods

MAb 13C03 (anti-human CD13, IgG1) was from Neomarkers, LabVision Corporation (Fremont, Calif.); mAb WM15 (anti-human CD13, IgG1) was from Pharmingen (San Diego, Calif.); Human recombinant TNF and NGR-TNF (consisting of human TNF₁₋₁₅₇ fused with the C-terminus of CNGRCG, SEQ ID NO: 271) were prepared by recombinant DNA technology and purified from E. coli cell extracts, according to Curnis et al. (2000). mAb 78 (IgG1) was obtained from Dr E. Barbanti (Pharmacia-Upjohn, Milan, Italy). MAb 78 is an anti-human TNF antibody able to form stable complexes with soluble TNF (K_{d}: 3.2×10⁻¹⁰ M) and to neutralize its interaction with membrane receptors (Barbanti et al., 1993).

Surgical specimens of human tissues (Bouin-fixed for 4-6 h, paraffin-embedded specimens 5-6 μm thick) were adsorbed on polylysine-coated slides. Antigens were detected using the avidin-biotin complex method. Tissue sections were rehydrated using xylenes and a graded alcohol series, according to standard procedures. Tissue sections were placed in a vessel containing 1 mM EDTA and boiled for 7 min using a microwave oven (1000 W). The vessel was then refilled with 1 mM EDTA and boiled again for 5 min. The tissue sections were left to cool and incubated in PBS containing 0.3% hydrogen peroxide for 15 min to quench endogenous peroxidase. The samples were then rinsed with PBS and incubated with 100-200 μl of PBS-BSA (1 h at room temperature) followed by the primary antibody or NGR-TNF/78 complex in PBS-BSA (overnight at 4° C.).

Complexes of human NGR-TNF and anti-TNF mAb 78 (termed NGR-TNF78) were prepared by incubating a mixture of 1 μg/ml NGR-TNF and 1 μg/ml mAb 78, both in PBS containing 2% BSA (PBS-BSA) for 20 min (20° C.). A mixture of TNF and mAb 78 (termed TNF/78) was prepared in the same way using human TNF instead of NGR-TNF. The slides were then washed 3 times (3 min each) with PBS and incubated with PBS-BSA containing 2% normal horse serum (PBS-BSA-NHS) (Vector Laboratories, Burlingame, Calif.) for 5 min. The solution was then replaced with 3 μg/ml biotinylated horse anti-mouse IgG (H+L) (Vector Laboratories, Burlingame, Calif.) in PBS-BSA-NHS and further incubated for 1 h at room temperature. The slides were washed again and incubated for 30 min with Vectastain Elite Reagent (Vector Laboratories, Burlingame, Calif.) diluted 1:100 in PBS. A tablet of 3,3′-diamino-benzidine-tetrahydrochloride (Merck, Darmstadt, Germany) was dissolved in 10 ml of deionized water containing 0.03% hydrogen peroxide, filtered through a 0.2 μm membrane and overlaid on tissue sections for 5-10 min. The slides were washed as above and counterstained with Harris' hematoxylin.

Results

A CD13 Isoform Associated with Tumor Vessels is a Receptor for NGR-TNF in Renal Cell Carcinoma

The expression of CD13 and the distribution of NGR binding sites were compared by immunohistochemistry. Sections of normal kidney and renal cell carcinoma were incubated with human NGR-TNF pre-complexed with the anti-human TNF mAb 78 (NGR-TNF/78). Controls with TNF/78 complexes or mAb 78 alone were also included. These complexes offer the advantage that they can be used as a single reagent in parallel with other antibodies. The staining patterns obtained with NGR-TNF/78 were very similar to those of WM15 and distinct from those of 13C03 (not shown). Like WM15, NGR-TNF/78 interacted with tumor associated vessels but not with the brush border of renal proximal tubule epithelial cells (not shown). No binding was observed with controls, such as TNF/78 or mAb 78 alone (not shown).

These results indicate that: a) the binding of mAb 78 to endogenous TNF is negligible; b) the binding of TNF/78 to TNF receptors is undetectable; c) the binding of NGR-TNF/78 depends on the interaction of the NGR domain with an NGR receptor. Accordingly, the binding of NGR-TNF/78 was completely inhibited by coincubation with an NGR-IFNγ conjugate (not shown). These and the above results suggest that the NGR-receptor and WM15 antigen co-localize in tumor associated vessels. They further show that immunohistochemistry with phage displaying targeting peptide sequences can be used to map the distribution of targeting peptide receptors in thin sections and to detect the presence of receptors in organ or tissue samples.

### Example 14

Identification of Targets in Tissue Sections: Angiostatin Targeting

The use of tissue thin sections for immunohistochemistry and detection of receptors, demonstrated in Example 13, was confirmed for angiostatin receptors. Angiostatin was incubated with tissue sections of metastitic human bone marrow. After washing, the tissue sections were developed with an anti-angiostatin Ab, followed by the corresponding secondary Ab conjugated to peroxidase. Tissue sections exhibited staining with a “vessel-like” structure. The staining was specific since only this structure and not other cells were stained. This result suggests that the angiostatin receptor is localized in the vasculature.

Materials and Methods

Anti-human Angiostatin were purchased (R&D Systems: AF226). rhAngiostatin was produced by EntreMed, Inc. (Rockville, Md.). Surgical specimens of human metastases (Bouin-fixed 4-6 h, paraffin-embedded sections, 5-6 μm thick) were adsorbed on polylysine-coated slides. Tissue slides were incubated with 100 μg/ml of rh-angiostatin. The slides were washed 3 times and incubated with anti-angiostatin antibody. The slides were washed again 3 times and bound antibodies were detected using an anti-goat peroxidase-conjugate antibody.

Results

Targeted Angiogenic Vasculature with Angiostatin.

Tissue sections stained with angiostatin binding exhibited a stained with “vessel-like” structure (not shown). The staining was specific since only this structure and not others were stained. Staining did not represent in situ angiostatin, since anti-angiostatin antibody by itself showed no staining of the samples (not shown).

These results confirm that angiostatin receptors are present in angiogenic tissues. They also confirm that targeting peptides or their endogenous analogs can be used in immunohistological staining to detect the presence of receptors for targeting peptide sequences in tissue thin sections.

All of the COMPOSITIONS, METHODS and APPARATUS disclosed and claimed herein can be made and executed without undue experimentation in light of the present disclosure. While the compositions and methods of this invention have been described in terms of preferred embodiments, it are apparent to those of skill in the art that variations may be applied to the COMPOSITIONS, METHODS and APPARATUS and in the steps or in the sequence of steps of the methods described herein without departing from the concept, spirit and scope of the invention. More specifically, it are apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

Anand-Apte B, Pepper M S, Voest E, Montesano R, Olsen B, Murphy G, Apte S S and Zetter B. Inhibition of angiogenesis by tissue inhibitor of metallopeinase-3. Invest. Opthamol. Vis. Sci.38: 817-823, 1997

Arap W, Pasqualini R, and Ruoslahti E. Chemotherapy targeted to tumor vasculature. Curr. Opin. Oncol., 1998b.

Arap, W., Pasqualini R., and Ruoslahti, E. Cancer treatment by targeted drug delivery to tumor vasculature. Science 279:377-380, 1998a.

Arap, W., Pasqualini, R. & Ruoslahti, E. Chemotherapy targeted to tumor vasculature. Curr Opin Oncol 10, 560-565 (1998).

Baichwal and Sugden, In: Gene Transfer, Kucherlapati R, ed., New York, Plenum Press, pp. 117-148, 1986.

Baldwin, R. W. et al. Monoclonal antibody-defined antigens on tumor cells. Biomembranes 11, 285-312 (1983).

Barany and Merrifield, The Peptides, Gross and Meienhofer, eds., Academic Press, New York, pp. 1-284, 1979.

Barbanti, E., Corti, A., Ghislieri, M., Casero, D., Rifaldi, B., Portello, C., Breme, U., Trizio, D., and Marcucci, F. Mode of interaction between tumor necrosis factor alpha and a monoclonal antibody expressing a recurrent idiotype. Hybridoma. 12: 1-13, 1993.

Bartlett, J. S., Kleinschmidt, J., Boucher, R. C. & Samulski, R. J. Targeted adeno-associated virus vector transduction of nonpermissive cells mediated by a bispecific Fab′gamma)2 antibody. Nat Biotechnol 17, 181-186, 1999.

BERGELSON, J. M., CUNNINGHAM, J. A., DROGUETT, G., KURT-JONES, E. A.,

KRITHIVAS, A., HONG, J. S., HORWITZ, M.S., CROWELL, R. L., and FINBERG, R. W. (1997). Isolation of a common receptor for coxsackie B viruses and adenoviruses 2 and 5. Science 275; 1320-1322.

Bielenberg, D. R., M. F. McCarty, C. D. Bucana, S. H. Yuspa, D. Morgan, J. M. Arbeit, L. M. Ellis, K. R. Cleary, and I. J. Fidler. 1999. Expression of interferon-beta is associated with growth arrest of murine and human epidermal cells. J Invest Dermatol 112:802-9.

Boehm T, Folkman J, Browder T, and O'Reilly M S. Antiangiogenic therapy of experimental cancer does not induce acquired drug resistance. Nature 390:404-407, 1997

Boon, T. & Old, L. J. Cancer Tumor antigens. Curr Opin Immunol 9,681-683 (1997).

Booth, S. N., et al., Cancer-associated proteins in effusion fluids. J Clin Pathol, 1977. 30: 53740.

Bossemeyer, D., Engh, R. A., Kinzel, V., Ponstingl, H. and Huber, R. Phosphotransferase and substrate binding mechanism of the cAMP-dependent protein kinase catalytic subunit from porcine heart as deduced from the 2.0 A structure of the complex with Mn²⁺ adenylyn imidiophosphate and inhibitor peptide PKI(5-24). EMBO J. 12:849-859, 1993.

Breborowicz, D., et al., The correlation of routine cytology with tile contents of carcinoembryonic antigen and alpha-fetoprotein in pleural and peritoneal effusions. Arch Geschwulstforsch, 1977. 47:231-5.

Brodt et. al, The role of marrow endothelium in the localization of metastastic cancer cells to bone. In Bone Metastasis-mechanisms and pathophysiology, pp17-23, 1996. (Orr and Singh, eds.)

Brooks P C, Clark R A, Cheresh D A. Requirement of vascular integrin αvβ3 for angiogenesis. Science 264:569-571, 1994a.

Brooks P C, Stromblad S, Klemle R, Visscher D, Sarkar F H, and Cheresh D A. Anti-integrin αvβ3 blocks human breast cancer growth and angiogenesis in human skin. J. Clin. Invest. 96:1815-1822, 1995.

Brooks, P. C. et al. Localization of matrix metalloproteinase MMP-2 to the surface of invasive cells by interaction with integrin alpha v beta 3. Cell 85, 683-693, 1996.

Brooks, P. C., Montgomery A. M., Rosenfeld, M., Reisfeld, R. A., Hu, T., Klier, G., and Cheresh D. A. Integrin αvβ3 antagonists promote tumor regression by inducing apoptosis of angiogenic blood vessels. Cell 79, 1157-1164, 1994b

Brousset, P., S. Chittal, D. Schlaifer, J. Icart, C. Payen, F. Rigal-Huguet, J. J. Voigt, and G. Delsol. 1991. Detection of Epstein-Barr virus messenger RNA in Reed-Sternberg cells of Hodgkin's disease by in situ hybridization with biotinylated probes on specially processed modified acetone methyl benzoate xylene (ModAMeX) sections. Blood 77:1781-6.

Burg M, Pasqualini R, Arap W, Stallcup W, and Ruoslahti E. Identification of NG2 proteoglycan-binding peptides that home to tumor neovasculature. Cancer Res 58:2869-2874, 1999.

Burg, M. A., Pasqualini, R., Arap, W., Ruoslahti, E. & Stallcup, W. B. NG2 proteoglycan-binding peptides target tumor neovasculature. Cancer Res 59, 2869-2874, 1999.

Campbell et al., Am. J. Pathol., 158:25-32, 2001.

Cao Y. O'Reilly M S. Marshall B. Flynn E. Ji R W and Folkman J. Expression of angiostatin cDNA in a murine fibrosarcoma suppresses primary tumor growth and produces long-term dormancy of metastases. J. Clin. Invest. 101:1055-1063, 1998.

Carter, H. B., Piantadosi, S. & Isaacs, J. T. Clinical evidence for and implications of the multistep development of prostate cancer. J Urol 143, 742-746 (1990).

Chang, K. L., and L. M. Weiss. 1996. The association of the Epstein-Barr virus with malignant lymphoma. Biomed Pharmacother 50:459-67.

Chen and Okayama, Mol. Cell Biol., 7:2745-2752, 1987.

Chen et al., J. Cell. Biochem., 78:404-416, 2000.

Chinni et al., Clin. Cancer Res. 3:1557-64, 1997.

Clark, E. A. and Brugge, J. S. Integrins and signal transduction pathways: the road taken. Science 268:233-238,1995.

Coffin, In: Virology, Fields et al., eds., Raven Press, New York, pp. 1437-1500, 1990.

Cortese, I. et al. Identification of peptides specific for cerebrospinal fluid antibodies in multiple sclerosis by using phage libraries. Proc Natl Acad Sci USA 93, 11063-11067 (1996).

Couch et al., Am. Rev. Resp. Dis., 88:394-403, 1963.

Coupar et al., Gene, 68:1-10, 1988.

Cox, D. R. Regression models and life tables. Journal of the Royal Statistical Society 74, 187-220 (1972).

Curiel, D. T. Strategies to adapt adenoviral vectors for targeted delivery. Ann N Y Acad Sci 886, 158-171, 1999.

Curnis, F., Sacchi, A., Borgna, L., Magni, F., Gasparri, A., and Corti, A. Enhancement of tumor necrosis factor alpha antitumor immunotherapeutic properties by targeted delivery to aminopeptidase N (CD13). Nat. Biotechnol. 18: 1185-90, 2000

Defilippi, P., Bozzo, C., Volpe, G., Romano, G., Venturino, M., Silengo, L. and Tarone, G. Integrin-mediated signal transduction in human endothelial cells: analysis of tyrosine phosphorylation events. Cell Adh. Commun. 87:75-86, 1994.

Delannet, M., Martin, F., Bossy, B., Cheresh, D. A., Reichardt, L. F. and Duband, J. L. Specific roles of the αvβ1, αvβ3, and αvβ5 integrins in avian neural crest cell adhesion and migration on vitronectin. Development. 120:2687-702, 1994.

Delpino et al., Mol. Membr. Biol. 15:21-26, 1998.

Dente, L., Vetriani,C., Zucconi, A., Pelicci, G., Lanfrancone, L., Pelicci, P. G. and Cesareni, G. Modified phage peptide libraries as a tool to study specificity of phosphorylation and recognition of tyrosine containing peptides. J. Mol. Biol. 269:694-703, 1997.

Derossi, D., Chassaing, G. and Prochiantz, A. Trojan peptides: the penetratin system for intracellular delivery. Trends Cell Biol. 8:84-87, 1998.

Derossi, D., Joliot, A. H., Chassaing, G. and Prochiantz, A. The third helix of Antennapedia homeodomain translocates through biological membranes. J. Biol. Chem. 269:10444-10450, 1994

DMITRIEV, I., KRASNYKH, V., MILLER, C. R., WANG, M., KASHENTSEV, A. E., MIKHEEVA, G., BELOUSOVA, N., and CURIEL, D. T. (1998). An adenovirus vector with genetically modified fibers demonstrates expanded tropism via utilization of a coxsackie virus and adenovirus receptor-independent cell entry mechanism. J. Virol. 72; 9706-9713.

Dorsett, B. H., et al., Isolation of tumor-specific antibodies from effusions of ovarian carcinomas. Int J Cancer, 1975. 16(5): p. 779-86

DOUGLAS, J. T., ROGERS, B. E., ROSENFELD, M. E., MICHAEL, S. I., FENG, M., and CURIEL, D. T. (1996). Targeted gene delivery by tropism-modified adenoviral vectors. Nature Biotechnol. 14; 1574-1578.

Dunn, I. S. Mammalian cell binding and transfection mediated by surface-modified bacteriophage lambda. Biochimie 78, 856-861, 1996.

Dybwad, A., Forre, O., Kjeldsen-Kragh, J., Natvig, J. B. & Sioud, M. Identification of new B cell epitopes in the sera of rheumatoid arthritis patients using a random nanopeptide phage library. Eur J Immunol 23, 3189-3193 (1993).

Eisen, T. et al. Continuous low dose Thalidomide: a phase II study in advanced melanoma, renal cell, ovarian and breast cancer. Br J Cancer 82, 812-817, 2000.

Ellerby H M, Arap W, Ellerby L, Kain R, Andrusiak R, Rio G, Krajeswki S. Lombardo C, Rao R, Ruoslahti E, Bredesen D, and Pasqualini R. Anti-cancer Activity of Targeted proapoptotic peptides. Nature Med 9:1032-1038, 1999

Enblad, G., K. Sandvej, E. Lennette, C. Sundstrom, G. Klein, B. Glimelius, and G. Pallesen. 1997. Lack of correlation between EBV serology and presence of EBV in the Hodgkin and Reed-Sternberg cells of patients with Hodgkin's disease. Int J Cancer 72:394-7.

Engelstädter M et al. Targeting human T cells by retroviral vectors displaying antibody domains selected from a phage display library. Hum Gene Ther. 2000; 11:293-303.

Engermnan, R. L. and Kern, T. S. (1986) Hyperglycemia as a cause of diabetic retinopathy. Metabolism 35(S1), 20-23.

Ferrara, N. and Davis-Smyth, T. (1997) The biology of vascular endothelial growth factor. Endocr. Rev., 18, 4-25.

Filardo, E. J. and Cheresh, D. A. A β turn in the cytoplasmic tail of the integrin αv subunit influences conformation and ligand binding of αvβ3. J. Biol. Chem. 269:4641-4647, 1994a.

Filardo, E. J. and Cheresh, D. A. A structural basis for bidirectional integrin signalling. Princess Takamatsu Symp. 24:106-117, 1994b.

Filardo, E. J., Brooks, P. C., Deming, S. L., Damsky, C. and Cheresh, D. A. Requirement of the NPXY motif in the integrin β3 subunit cytoplasmic tail for melanoma cell migration in vitro and in vivo. J. Cell Biol. 130:441-450, 1995.

Folkman J. Addressing tumor blood vessels. Nature Biotechnol. 15: 510, 1997.

Folkman J. Angiogenesis in cancer, vascular, rheumatoid and other disease. Nature Med 1:27-31, 1995

Folkman, J. Antiangiogenic gene therapy. Proc Natl Acad Sci USA 95, 9064-9066, 1998.

Friedlander M, Brooks P C, Sharffer R W, Kincaid C M, Varner J A, and Cheresh D A. Definition of two angiogenic pathways by distinct αv integrins. Science, 270: 1500-1502, 1995.

Friedlander M, Theesfeld C L, Sugita M, Fruttiger M, Thomas M A, Chang S, Cheresh D A. Involvement of integrins αvβ3 and αvβ5 in ocular neovascular diseases. Proc. Natl. Acad. Sci. USA 93:9764-9769, 1996.

Friedmann, Science, 244:1275-1281, 1989.

Frisch S M. And Ruoslahti E. Integrins and anoikis. Cur. Opin. in Cell Biol. 9:701-706, 1997.

Furuya et al., Cancer Res. 54:6167-75, 1994.

Ghosh-Choudhury et al., EMBO J., 6:1733-1739, 1987.

Gingrich J R, Barrios R J, Morton R A, Boyce B F, DeMayo F J, Finegold M J, Angelopoulou R, Rosen J M and Greenberg N M. Metastatic prostate cancer in a transgenic mouse. Cancer Res. 56:4096-4102, 1996.

Girod A et al. Genetic capsid modifications allow efficient re-targeting of adeno-associated virus type 2. Nat Med 1999; 5: 1052-1056.

Gold R. Differenitation between Cellular Apoptosis ad Necrosis by the Combined Use of In Situ Tailing Translation Techniques. Lab. Invest. 71: 219, 1994

Goldman C K et al. Targeted gene delivery to Karposi's sarcoma cells via the fibroblast growth factor receptor. Cancer Res 1997; 57: 1447-1451.

GOLDMAN, C. K., ROGERS, B. E., DOUGLAS, J. T., SOSNOWSKI, B. A., YING, W., SIEGAL, G. P., BAIRD, A., CAMPAIN, J. A., and CURIEL, D. T. (1997). Targeted gene delivery to Karposi's sarcoma cells via the fibroblast growth factor receptor. Cancer Res. 57; 1447-1451.

Gomez-Foix et al., J. Biol. Chem., 267:25129-25134, 1992.

Gopal, Mol. Cell Biol., 5:1188-1190, 1985.

Grace, M. J., L. Xie, M. L. Musco, S. Cui, M. Gurnani, R. DiGiacomo, A. Chang, S. Indelicato, J. Syed, R. Johnson, and L. L. Nielsen. 1999. The use of laser scanning cytometry to assess depth of penetration of adenovirus p53 gene therapy in human xenograft biopsies. Am J Pathol 155:1869-78.

Graham and Prevec, In: Methods in Molecular Biology: Gene Transfer and Expression Protocol, E. J. Murray, ed., Humana Press, Clifton, N.J., 7:109-128, 1991.

Graham and van der Eb, Virology, 52:456-467, 1973.

Graham et al., J. Gem Virol., 36:59-72, 1977.

Gram, H., Schmitz, R., Zuber, J. F. and Baumann, G. Identification of phosphopeptide ligands for Src-homology 2 (SH2) domain of Grb2 by phage display. Eur. J. Biochem. 246:633-637, 1997.

Greenberg N M, DeMayo F, Finegold M J, Medina D, Tilley W D, Aspinall J O, Cunha G R, Donjacour A A, Matusik R J and Rosen J M. Prostate cancer in a transgenic mouse. Proc. Natl. Acad. Sci. USA 92:3439-3443, 1995.

Griscelli F. Li H. Bennaceur-Griscelli A. Soria J. Opolon P. Soria C. Perricaudet M. Yeh P and Lu H. Angiostatin gene transfer: inhibition of tumor growth in vivo by blockage of endothelial cell proliferation associated with a mitosis arrest. Proc. Natl. Acad. Sci. USA 95:6367-72, 1998

Grunhaus and Horwitz, Seminar in Virology, 3:237-252, 1992.

Gunge et al., Mol. Gen. Genet. 263:846-853, 2000.

Hall, H., Williams, E J., Moore, S E., Walsh, F S., Prochiantz, A. and Doherty, P. Inhibition of FGF-stimulated phosphatidylinositol hydrolysis and neuron outgrowth by a cell-membrane permeable phosphopeptide. Current Biology, 6:580-587, 1996.

Hammes H P, Brownlee M, Jonczyk A, Sutter A, and Preissner K T. Subcutaneous injection of a cyclic peptide antagonist of vitronectin receptor-type integrins inhibits retinal neovascularization. Nature Med. 2: 529-533, 1996.

Hanahan, D. and Folkman, J. (1996) Patterns and Emerging Mechanisms of the Angiogenic Switch during Tumorogenesis. Cell, 86, 353-364.

Hansen, A. S., Norén, O., Sjöstrom, H. and Wedelin, O. (1993) A mouse aminopeptidase-N is a marker for antigen presenting cells and apears to be co-expressed with major histocompatibility complex class II molecules. Eur. J. Immunol., 23, 2358-64.

HARLOW, E., and LANE, D. (1988). Antibodies: A Laboratory Manual (Cold Spring Harbor Laboratory Press, New York, N.Y.).

Hart S L et al. Cell binding and internalization by filamentous phage displaying a cyclic Arg-Gly-Asp-containing peptide. J. Biol. Chem. 269, 12468-12474,1994

Hemler, M., Weitzman, J., Pasqualini, R., Kawaguchi, S., Kassner, P. and Berdichevsky, F. Structure, biochemical properties, and biological functions of integrin cytoplasmic domains. In: Integrins: The Biological Problems (ed Yoshi Takada) CRC Press, Inc., Boca Raton, Fla., USA; pp.1-35, 1994.

Hendrix R W. Evolution: the long evolutionary reach of viruses. Current Biol. 9:914-917, 1999.

HENRY, L., XIA, D., WILKE, M., DEISENHOFER, J., and GERARD, R. (1994). Characterization of the knob domain of the adenovirus type 5 fiber protein expressed in E. coli. J. Virol. 68; 5239-5246.

Herbst, H., E. Steinbrecher, G. Niedobitek, L. S. Young, L. Brooks, N. Muller-Lantzsch, and H. Stein. 1992. Distribution and phenotype of Epstein-Barr virus-haboring cells in Hodgkin's disease. Blood 80:484-91.

Herbst, H., F. Dallenbach, M. Hummel, G. Niedobitek, S. Pileri, N. Muller-Lantzsch, and H. Stein. 1991. Epstein-Barr virus latent membrane protein expression in Hodgkin and Reed-Sternberg cells. Proc Natl Acad Sci USA 88:4766-70.

Hermonat and Muzycska, Proc. Natl. Acad Sci. USA, 81:6466-6470, 1984.

Herndier B G, Werner A, Arnstein P, Abbey N W, Demartis F, Cohen R L, Shuman M A and Levy, J A Characterization of a human Kaposi's sarcoma cell line that induces angiogenic tumors in animals. AIDS 8:575-581, 1996.

Hersdorffer et al., DNA Cell Biol., 9:713-723, 1990.

Herz and Gerard, Proc. Natl Acad. Sci. USA, 90:2812-2816, 1993.

HEYWOOD, S. P., and HOOPER, N. M. (1995). Development and application of a fluorometric assay for mammalian membrane dipeptidase. Anal. Biochem. 226; 10-14.

Hill, R., et al., Isolation of tumour-associated immunoglobulins from ascitic fluid. Br J Cancer, 1978. 38:1547

HONG, S. S., GALAUP, A., PEYTAVI, R., CHAZAL, N., and BOULANGER, P. A. (1999). Enhancement of adenovirus-mediated gene delivery by use of an oligopeptide with dual binding specificity. Hum. Gene Ther. 10; 2577-2586.

HONG, S. S., KARYAN, L., TOURNIER, J., CURIEL, D. T., and BOULANGER, P. A. (1997). Adenovirus type 5 fiber knob binds to MHC class I alpha-2 domain at the surface of human epithelial and B lymphoblastoid cells. EMBO J. 16; 2294-2306.

Horwich, et al., J. Virol., 64:642-650, 1990.

Hughes et al., Cancer Res. 49:4452-54, 1989

Hynes, R. O. Integrins: versatility, modulation and signaling in cell adhesion. Cell 69:11-25, 1992.

Ivanenkov, V., Felici, F. & Menon, A. G. Uptake and intracellular fate of phage display vectors in mammalian cells. Biochim Biophys Acta 1448, 450-462, 1999a.

Ivanenkov, V. V., Felici, F. & Menon, A. G. Targeted delivery of multivalent phage display vectors into mammalian cells. Biochim Biophys Acta 1448, 463-472, 1999b. J. Natl. Cancer Inst. 90:273-286, 1998.

Jarrett, A. F., A. A. Armstrong, and E. Alexander. 1996. Epidemiology of EBV and Hodgkin's lymphoma. Ann Oncol 7:5-10.

Jarrett, R. F., and J. MacKenzie. 1999. Epstein-Barr virus and other candidate viruses in the pathogenesis of Hodgkin's disease. Semin Hematol 36:260-9.

Johnson et al., “Peptide Turn Mimetics” in BIOTECHNOLOGY AND PHARMACY, Pezzuto et al., Eds., Chapman and Hall, New York (1993).

Joliot, A. H. Triller, A., Volovitch, M. Pernelle, C., and Prochiantz, A. alpha-2,8-Polysialic acid is the neuronal surface receptor of antennapedia homeobox peptide. New Biol. 3:1121-1131, 1991a.

Joliot, A. H., Pernelle, C., Deagostini-Bazin, H. and Prochiantz, A. Antennapedia homeobox peptide regulates neural morphogenesis Proc. Natl. Acad. Sci. U.S.A. 88:1864-1868, 1991b.

Jones and Shenk, Cell, 13:181-188, 1978.

Kaplan, E. L. a. M., P. Nonparametric estimation from incomplete observations. Journal of the American Statistical Association 53, 457481 (1958).

Karlsson et al., EMBO J., 5:2377-2385, 1986.

Kasono, K. et al. Selective gene delivery to head and neck cancer cells via an integrin targeted adenoviral vector. Clin Cancer Res 5, 2571-2579, 1999.

Kassner, P. D., Burg, M. A., Baird, A. & Larocca, D. Genetic selection of phage engineered for receptor-mediated gene transfer to mammalian cells. Biochem Biophys Res Commun 264, 921-928, 1999.

Khoo Sk, H. R., and Mackay E V, Detection of carcinoembryonic antigen and alphafetoprotein in serum and ascitic fluid from patients with ovarian cancer. Aust. N. Z. J. Obstet. Gynaecol., 1977. 17:94-97

Kiang et al., Chin. J. Physiol. 40:213-219, 1997

Klemke, R. L., Yebra, M., Bayna, E. M. and Cheresh, D. A. Receptor tyrosine kinase signaling required for integrin αvβ5 -directed cell motility but not adhesion on vitronectin. J. Cell Biol. 127:859-866, 1994.

Koivunen E, Arap W, Valtanen H, Rainisalo A, Gahmberg C G, Salo T, Konttinen Y T, Sorsa T, Ruoslahti E, Pasqualini R. Tumor targeting with a selective gelatinase inhibitor. Nature Biotechnol 17:768-774, 1999

Koivunen E, Gay D A and Ruoslahti E. Selection of peptides binding to the α5β1 integrin from phage display library. J. Biol. Chem. 268:20205-20210, 1993.

Koivunen E, Wang B, and Ruoslahti E. Phage display libraries displaying cyclic peptides with different ring sizes: ligand specificities of the RGD-directed integrins. BioTechnology 13:265-270, 1995.

Koivunen, E. et al. Integrin-binding peptides derived from phage display libraries. Methods Mol Biol 129, 3-17 (1999).

Kolanus, W. and Seed, B. Integrins and inside-out signal transduction: converging signals from PKC and PIP3. Curr. Opin. Cell Biol. 9:725-731, 1997.

Kolonin M G. Finley R L Jr. Targeting cyclin-dependent kinases in Drosophila with peptide aptamers. Proc. of the Natl. Acad. of Sci. USA.95:14266-71, 1998.

Kong H L and Crystal R G. Gene therapy strategies for tumor antiangiogenesis.

Kouzmitcheva G. A. et al. Identifying diagnostic peptides for lyme disease through epitope discovery. Clin Diagn Lab Immunol 8, 150-60 (2001).

KOZARSKY, K., JOOSS, K., DUNAHEE, M., STRAUSS, J. F., and WILSON, J. M. (1996). Effective treatment of familial hypercholesterolaemia in the mouse model using adenovirus-mediated transfer of the VLDL receptor gene. Nat. Genet. 13; 54-62.

KRASNYKH, V., DMITRIEV, I., MIKHEEV, A. G., MILLER, C. R., BELOUSOVA, N., and CURIEL, D. T. (1998). Characterization of an adenovirus vector containing a heterologous peptide epitope in the HI loop of the fiber knob. J. Virol. 72; 1844-1852.

KRASNYKH, V., MIKHEEVA, G. V., DOUGLAS, J. T., and CURIEL, D. T. (1996). Generation of recombinant adenovirus vectors with modified fibers for altering viral tropism. J. Virol. 70; 6839-6846.

Lane T. Shah J. Clinical features and management of benign prostatic hyperplasia. Hospital Medicine. 60(10):705-9, 1999.

Larocca D et al. Gene transfer to mammalian cells using genetically targeted filamentous bacteriophage. FASEB J 1999; 13:727-734.

Larocca, D., Witte, A., Johnson, W., Pierce, G. F. & Baird, A. Targeting bacteriophage to mammalian cell surface receptors for gene delivery. Hum Gene Ther 9, 2393-2399, 1998.

Le Gal La Salle et al., Science, 259:988-990, 1993.

Le Roux, I., Joliot, A. H., Bloch-Gallego, E., Prochiantz, A. and Volovitch, M. Neurotrophic activity of the Antennapedia homeodomain depends on its specific DNA-binding properties. Proc. Natl. Acad. Sci. U.S.A. 90:9120-9124, 1993

Levrero et al., Gene, 101: 195-202, 1991.

Lewis, J. M., Cheresh, D. A. and Schwartz, M. A. Protein kinase C regulates αvβ5-dependent cytoskeletal associations and focal adhesion kinase phosphorylation. J. Cell Biol. 134:1323-1332, 1996.

Lin, T. H., Aplin, A. E., Shen, Y., Chen Q., Schaller, M. D., Romer L., Aukhil, I. and Juliano, R. L. Integrin-mediated activation of MAP kinase is independent of FAK: evidence for dual integrin signalling pathways in fibroblast. J. Cell Biol. 136:1385-1395, 1997.

Longhurst, C. M. and Jennings, L. K. Integrin-mediated signal transduction. Cell Mol. Life Sci. 54:514-526, 1998.

Look A T, Ashmun R A, Shapiro L H and Peiper S C. Human myeloid plasma membrane glycoprotein CD13 (gp150) is identical to aminopeptidase N. J. Clin. Invest. 83:1299-1307, 1989.

LOUIS, N., FENDER, P., BARGE, A., KITS, P., and CHROBOCZEK, J. (1994). Cell-binding domain of adenovirus serotype 2 fiber. J. Virol. 68; 4104-4106.

Lunardi, C. et al. Systemic sclerosis immunoglobulin G autoantibodies bind the human cytomegalovirus late protein UL94 and induce apoptosis in human endothelial cells [In Process Citation]. Nat Med 6, 1183-1186 (2000).

Lynch, C. M. et al. Adeno-associated virus vectors for vascular gene delivery. Circ Res 80,497-505, 1997.

Lyons, S. F., and D. N. Liebowitz. 1998. The roles of human viruses in the pathogenesis of lymphoma. Semin Oncol 25:461-75.

MacGregor, G. R. & Caskey, C. T. Construction of plasmids that express E. coli beta-galactosidase in mammalian cells. Nucleic Acids Res 17, 2365, 1989.

Maggino, T. and A. Gadducci, Serum markers as prognostic factors in epithelial ovarian cancer: an overview. Eur J Gynaecol Oncol, 2000. 21:649

Mahboubi et al, J. Immunol. 164:3837-3846, 2000.

Mann et al., Cell, 33:153-159, 1983.

Markowitz et al., J. Virol., 62:1120-1124, 1988.

Martin F et al. Retrovirus targeting by tropism restriction to melanoma cells. J Virol 1999; 73: 6923-6929.

Martiny-Baron G, and Marme D. VEGF-mediated tumor angiogenesis: a new target for cancer therapy. Curr. Opin. Biotechnol. 6:675-680, 1995.

Mennuni, C. et al. Selection of phage-displayed peptides mimicking type 1 diabetes-specific epitopes. J Autoimmun 9,431-436 (1996).

Merrifield, Science, 232: 341-347, 1986

Meyer, T. and G. J. Rustin, Role of tumour markers in monitoring epithelial ovarian cancer. Br J Cancer, 2000. 82:1535-8

MICHAEL, S. I., HONG, J. S., CURIEL, D. T., and ENGLER, J. A. (1995). Addition of a short peptide ligand to the adenovirus fiber protein. Gene Ther. 2; 660-668.

Mikolajczyk S D. Millar L S. Wang T J. Rittenhouse H G. Marks L S. Song W. Wheeler T M. Slawin K M. A precursor form of prostate-specific antigen is more highly elevated in prostatecancer compared with benign transition zone prostate tissue. Cancer Research. 60(3):756-9, 2000.

Miller C R et al. Differential susceptibility of primary and established human glioma cells to adenovirus infection: targeting via the epidermal growth factor receptor achieves fiber receptor independent gene transfer. Cancer Res 1998; 58: 5738-5748.

Motti, C. et al. Recognition by human sera and immunogenicity of HBsAg mimotopes selected from an M13 phage display library. Gene 146, 191-198 (1994).

Mulligan, Science, 260:926-932, 1993.

Mustonen T and Alitalo K. Endothelial receptor tyrosine kinases involved in angiogenesis. J. Cell Biol. 129:895-898, 1995.

Muzyczka N. Adeno-associated virus (AAV) vectors: will they work? J. Clin. Invest. 94:1351, 1994

Nicolas and Rubinstein, In: Vectors: A survey of molecular cloning vectors and their uses, Rodriguez and Denhardt, eds., Stoneham: Butterworth, pp. 494-513, 1988.

Nicolau et al., Methods Enzymol., 149:157-176, 1987.

Old, L. J. Cancer immunology: the search for specificity—G. H. A. Clowes Memorial lecture. Cancer Res 41, 361-375 (1981).

Olofsson, B. Jeltsch, M., Eriksson, U. and Alitalo, K. (1999) Current Biology of VEGF-B and VEGF-C. Curr Op Biotechnol, 10, 528-535.

Olofsson, B., Pajusola, K., Kaipainen, A., Euler, G., Joukov, V., Saksela, O., Orpana, A., Pettersson, R. F., Alitalo, K. and Eriksson, U. (1996) Vascular Endothelial Growth factor B, a novel growth factor for endothelial cells. Proc Natl Acad Sci USA, 93, 2576-2581.

Owens, G. P., R. A. Williamson, M. P. Burgoon, O. Ghausi, D. R. Burton, and D. H. Gilden. 2000. Cloning the antibody response in humans with chronic inflammatory disease: immunopanning of subacute sclerosing panencephalitis (SSPE) brain sections with antibody phage libraries prepared from SSPE brain enriches for antibody recognizing measles virus antigens in situ. J Virol 74:1533-7.

Pallesen, G., S. J. Hamilton-Dutoit, M. Rowe, and L. S. Young. 1991. Expression of Epstein-Barr virus latent gene products in tumour cells of Hodgkin's disease [see comments]. Lancet 337:320-2.

Paskind et al., Virology, 67:242-248, 1975.

Pasqualini R and Ruoslahti E. Organ targeting in vivo using phage display peptide libraries. Nature 380:364-366, 1996.

Pasqualini R, Koivunen E, and Ruoslahti E. A peptide isolated from phage display libraries is a structural and functional mimic of an RGD-binding site on integrins. J. Cell Biol. 130:1189-1196, 1995.

Pasqualini R, Koivunen E, and Ruoslahti E. αv integrins as receptors for tumor targeting by circulating ligands. Nature Biotechnol 15:542-546, 1997

Pasqualini, R. and Hemler, M. E. Contrasting roles for integrin b1 and b5 cytoplasmic domains in subcellular localization, cell proliferation, and cell migration. J. Cell Biol. 125:447-60, 1994.

Pasqualini, R. Vascular Targeting with Phage Display Peptide libraries. The Quart. J. Nucl. Med. 43:159-162, 1999.

Pasqualini, R., Arap W., Koivunen, E., Kain, R., Lahdenranta, J., Shapiro, L., Sakamoto, M., Stryn, A. and Ruoslahti, E. Aminopeptidase N is a receptor for tumor-homing peptides and a target for inhibiting angiogenesis. Cancer Res. 60: 722-727, 2000.

Pelleymounter et al. Effects of the obese gene product on body weight regulation in ob/ob mice. Science 269: 540-543, 1994.

Pereboeva, L. A., A. V. Pereboev, and G. E. Morris. 1998. Identification of antigenic sites on three hepatitis C virus proteins using phage-displayed peptide libraries. J Med Virol 56:105-11.

Pereboeva, L. A., A. V. Pereboev, L. F. Wang, and G. E. Morris. 2000. Hepatitis C epitopes from phage-displayed cDNA libraries and improved diagnosis with a chimeric antigen. J Med Virol 60:144-51.

Potter et al., Proc. Nat. Acad. Sci. USA, 81:7161-7165, 1984.

Poul, M. A. & Marks, J. D. Targeted gene delivery to mammalian cells by filamentous bacteriophage. J Mol Biol 288, 203-211, 1999.

Prezzi, C. et al. Selection of antigenic and immunogenic mimics of hepatitis C virus using sera from patients. J Immunol 156, 4504-4513 (1996).

Prezzi, C., M. Nuzzo, A. Meola, P. Delmastro, G. Galfre, R. Cortese, A. Nicosia, and P. Monaci. 1996. Selection of antigenic and immunogenic mimics of hepatitis C virus using sera from patients. J Immunol 156:450413.

PRICE, J. E., POLYZOS, A., ZHANG, R. D., and DANIELS, L. M. (1990). Tumorigenicity and metastasis of human breast carcinoma cells lines in nude mice. Cancer Res. 50; 717-721.

Puntoriero, G. et al. Towards a solution for hepatitis C virus hypervariability: mimotopes of the hypervariable region 1 can induce antibodies cross-reacting with a large number of viral variants. Embo J 17, 3521-3533 (1998).

Racher et al., Biotechnology Techniques, 9:169-174, 1995.

Ragot et al., Nature, 361:647-650, 1993.

Rajotte D and Ruoslahti E. Membrane dipeptidase is the receptor for a lung-targeting peptide identified by in vivo phage display. J Biol Chem 274:11593-11598, 1999

Rajotte D, Arap W, Hagedorn M, Koivunen E, Pasqualini R, and Ruoslahti E. Molecular heterogeneity of the vascular endothelium revealed by in vivo phage display. J Clin Invest 102:430-437, 1998

Rak J W, St. Croix B D, and Kerbel R S. Consequences of angiogenesis for tumor progression, metastasis and cancer. Anticancer Drugs 6:3-18, 1995.

Razzaque, A., Y. Francillon, P. N. Jilly, and F. Varricchio. 1996. Detection of human herpesvirus 6 sequences in lymphoma tissues by immunohistochemistry and polymerase chain reactions. Cancer Lett 106:221-6.

Remington's Pharmaceutical Sciences, 15th ed., pp. 1035-1038 and 1570-1580.

Renan, Radiother. Oncol., 19:197-218, 1990.

Renata Pasqualini, W. A., Daniel Rajotte, and Erkki Ruoslahti. in Phage Display: A Laboratory manual (ed. Carlos F. Barbas III, D. R. B., Jamie K. Scott, and Gregg J. Silverman) 22.21-22.24 (Cold Spring Harbor Laboratory Press, New York, 2001).

Rich et al., Hum. Gene Ther., 4:461-476, 1993.

Ridgeway, In: Vectors: A Survey of Molecular Cloning Vectors and Their Uses, Rodriguez et al., eds., Stoneham: Butterworth, pp. 467492, 1988.

Rippe et al., Mol. Cell Biol., 10:689-695, 1990.

ROELVINK, P. W., LEE, G. M., EINFELD, D. A., KOVESDI, I., and WICKHAM, T. J. (1999). Identification of a conserved receptor-binding site on the fiber proteins of CAR-recognizing adenoviridae. Science 286; 1568-1571.

ROMANCZUK, H., GALER, C. E., ZABNER, J., BARSOMIAN, G., WADSWORTH, S. C., and O'RIORDAN, C. R. (1999). Modification of an adenoviral vector with biologically selected peptides: a novel strategy for gene delivery to cells of choice. Hum. Gene Ther. 10; 2615-2626.

Rosenfeld et al., Cell, 68:143-155, 1992.

Rosenfeld et al., Science, 252:431-434, 1991.

Rowley, M. J. et al. Prediction of the immunodominant epitope of the pyruvate dehydrogenase complex E2 in primary biliary cirrhosis using phage display. J Immunol 164, 3413-3419 (2000).

Ruoslahti E. RGD and other sequence recognition sequences for integrins. Annu. Rev. Cell Dev. Biol. 12:697-715, 1996

Sahin, U. et al. Human neoplasms elicit multiple specific immune responses in the autologous host. Proc Natl Acad Sci USA 92, 11810-11813 (1995).

Sahin, U., Tureci, O. & Pfreundschuh, M. Serological identification of human tumor antigens. Curr Opin Immunol 9, 709-716 (1997).

Scala, G. et al. Selection of H[V-specific immunogenic epitopes by screening random peptide libraries with HIV-1-positive sera. J Immunol 162, 6155-6161 (1999).

Schlingemann R O, Rietveld F J, de Waal R M, Ferrone S, Ruiter D J. Expression of the high molecular weight melanoma-associated antigen by pericytes during angiogenesis in tumors and in healing wounds. Am. J. Pathol. 136:1393-1405, 1990.

Schmitz, R., Baumann, G. and Gram, H. Catalytic specificity of phosphotyrosine kinase Blk, Lyn, c-Src and Syk as assessed by phage display J. Mol. Biol. 260: 664-677, 1996.

Shattil, S. J. and Ginsberg, M. H. Perspectives series: cell adhesion in vascular biology. Integrin signaling in vascular biology. J. Clin. Invest. 100:1-5, 1997.

Short S M, Talbott G A and Juliano R L. Integrin-mediated Signaling Events in Human Endothelial Cells. Mol. Biol. Cell 9: 1969-1980, 1998

Silverstein, JCI 74:1625-1633, 1984

Smith G. P. Surface presentation of protein epitopes using bacteriophage expression system. Curr Opin Biotechnol 2, 668-73 (1991).

Smith G P, and Scott J K. Libraries of peptides and proteins displayed in filamentous phage. Meth. Enzymol. 21:228-257, 1993.

Smith G P, and Scott J K. Searching for peptide ligands with an epitope library. Science 228:1315-1317, 1985

Smith, D. B., and K. S. Johnson. 1988. Single-step purification of polypeptides expressed in Escherichia coli as fusions with glutathione S-transferase. Gene 67:31-40.

Smith, G. P. 1985. Filamentous fusion phage: novel expression vectors that display cloned antigens on the virion surface. Science 228:1315-7.

Smith, G. P. Surface presentation of protein epitopes using bacteriophage expression systems. Curr. Opin. Biotechnol. 2:668-673, 1991.

Solowska J, Edelman J M, Albelda S M and Buck C A. (1991) Cytoplasmic and transmembrane domains of integrin β1 and β3 subunits are functionally interchangeable. J. Cell Biol. 114: 1079-1088.

Staratschek-Jox, A., S. Kotkowski, G. Belge, T. Rudiger, J. Bullerdiek, V. Diehl, and J. Wolf. 2000. Detection of Epstein-Barr virus in Hodgkin-Reed-Sternberg cells : no evidence for the persistence of integrated viral fragments inLatent membrane protein-1 (LMP-1)-negative classical Hodgkin's disease. Am J Pathol 156:209-16.

Sternberg, N. & Hoess, R. H. Display of peptides and proteins on the surface of bacteriophage lambda. Proc Natl Acad Sci USA 92, 1609-1613, 1995.

Stewart and Young, Solid Phase Peptide Synthesis, 2d. ed., Pierce Chemical Co., 1984.

Stoeckle et al., Mol. Cell Biol. 8:2675-80, 1988.

Stratford-Perricaudet and Perricaudet, In: Human Gene Transfer, O. Cohen-Haguenauer et al., eds., John Libbey Eurotext, France, pp. 51-61, 1991.

Stratford-Perricaudet et al., Hum. Gene. Ther., 1:241-256, 1990.

Tam et al., J. Am. Chem. Soc., 105:6442, 1983.

Tanaka T, Cao Y, Folkman J and Fine H A. Viral vector-targeted antiangiogenic gene therapy utilizing an angiostatin complementary DNA. Cancer Res. 58:3362-3369, 1998.

Temin, In: Gene Transfer, Kucherlapati R, ed., New York, Plenum Press, pp. 149-188, 1986.

Theodore, L., Derossi, D., Chassaing, G., Llirbat, B., Kubes, M., Jordan, P., Chneiweiss, H., Godement, P., and Prochiantz, A. Intraneuronal delivery of protein kinase C pseudosubstrate leads to growth cone collapse. J. Neurosci. 15:7158-7167, 1995.

Tischer, E., Mitchell, R., Hartman, T., Silvia, M., Gospodarowicz, D., Fiddes, J. C. and Abraham, J. (1991) the human Gene for Vascular Endothelial Growth Factor. J. Biol. Chem., 226, 11947-11954.

Top et al., J. Infect. Dis., 124:155-160, 1971.

Triantafilou et al., Hum. Immunol. 62:764-770, 2001.

Tureci, O., Sahin, U. & Pfreundschuh, M. Serological analysis of human tumor antigens: molecular definition and implications. Mol Med Today 3, 342-349 (1997).

Tur-Kaspa et al., Mol. Cell Biol., 6:716-718, 1986.

U.S. Pat. No. 3,817,837

U.S. Pat. No. 3,850,752

U.S. Pat. No. 3,939,350

U.S. Pat. No. 3,996,345

U.S. Pat. No. 4,275,149

U.S. Pat. No. 4,277,437

U.S. Pat. No. 4,366,241

U.S. Pat. No. 4,472,509

U.S. Pat. No. 5,021,236

U.S. Pat. No. 5,206,347

U.S. Pat. No. 5,223,409

U.S. Pat. No. 5,401,511

U.S. Pat. No. 5,603,872

U.S. Pat. No. 5,622,699

U.S. Pat. No. 5,889,155

U.S. Pat. No. 6,068,829

Varmus et al., Cell, 25:23-36, 1981.

Veikkola, T. and Alitalo, K. (1999) VEGFs, receptors and angiogenesis. Seminar Cancer Bio.l, 9, 211-220.

VIGNE, E., MAHFOUZ, I., DEDIEU, J. F., BRIE, A., PERRICAUDET, M., and YEH, P. (1999). RGD inclusion in the hexon monomer provides adenovirus type 5-based vectors with a fiber knob-independent pathway for infection. J. Virol. 73; 5156-5161.

Vu, T. H. et al. MMP-9/gelatinase B is a key regulator of growth plate angiogenesis and apoptosis of hypertrophic chondrocytes. Cell 93, 411-422, 1998.

Vuori K. Ruoslahti E. Association of insulin receptor substrate-1 with integrins. Science 266:1576-1578, 1994

WATKINS, S. J., MESYANZHNOV, V. V., KUROCHKINA, L. P., and HAWKINS, R. E. (1997). The adenobody approach to viral targeting—specific and enhanced adenoviral gene delivery. Gene Ther. 4; 1004-1012.

Watson C A, Camera-Benson L, Palmer-Croker R and Pober J S. Variability among human umbilical vein endothelial cell cultures. Science 268: 447448, 1995.

Weiss, L. M., J. G. Strickler, R. A. Warnke, D. T. Purtilo, and J. Sklar. 1987. Epstein-Barr viral DNA in tissues of Hodgkin's disease. Am J Pathol 129:86-91

Weiss, L. M., Y. Y. Chen, X. F. Liu, and D. Shibata. 1991. Epstein-Barr virus and Hodgkin's disease. A correlative in situ hybridization and polymerase chain reaction study. Am J Pathol 139:1259-65.

Weitzman M D, Wilson J M and Eck S L. Adenovirus vectors in cancer gene therapy. In: Gene Therapy and Vector Systems 2: 17-25, 1997.

Wells, J. A. and Lowman, H. B. Rapid evolution of peptide and protein binding properties in vitro. Curr. Opin. Biotechnol. 3:355-362, 1992.

Wickham T J. Haskard D. Segal D. Kovesdi I. Targeting endothelium for gene therapy via receptors up-regulated during angiogenesis and inflammation. Cancer Immunol. Immunother. 45:149-151, 1997c.

Wickham, T. J. Targeting adenovirus. Gene Ther 7, 110-114, 2000.

WICKHAM, T. J., CARRION, M. E., and KOVESDI, I. (1995). Targeting of adenovirus penton base to new receptors through replacement of its RGD motif with other receptor-specific peptide motifs. Gene Ther. 2; 750-756.

WICKHAM, T. J., LEE, G., TITUS, I., SCONOCCHIA, G., BAKACS, T., KOVESDI, I, and SEGAL, D. (1997a). Targeted adenovirus-mediated gene delivery to T-cells via CD3. J. Virol. 71; 7663-7669.

WICKHAM, T. J., MATHIAS, P., CHERESH, D. A., and NEMEROW, G. R. (1993). Integrins alpha v beta 3 and alpha v beta 5 promote adenovirus internalization but not attachment. Cell 73; 309-319.

WICKHAM, T. J., ROELVINK, P. W., BROUGH, D. E., and KOVESDI, I. (1996b). Adenovirus targeted to heparan-containing receptors increases its gene delivery efficiency to multiple cell types. Nature Biotechnol. 14; 1570-1573.

WICKHAM, T. J., SEGAL, D. M., ROELVINK, P. W., CARRION, M. E., LIZONOVA, A., LEE, G. M., and KOVESDI, L. (1996a). Targeted adenovirus gene transfer to endothelial and smooth muscle cells by using bispecific antibodies. J. Virol. 70; 6831-6838.

WICKHAM, T. J., TZENG, E., SHEARS II, L. L., ROELVINK, P. E., LI, Y., LEE, G. M., BROUGH, D. E., LIZONOVA, A., and KOVESDI, I. (1997b). Increased in vitro and in vivo gene transfer by adenovirus vectors containing chimeric fiber proteins. J. Virol. 71; 8221-8229.

Wong et al., Gene, 10:87-94, 1980.

Wu and Wu, Biochemistry, 27: 887-892, 1988.

Wu and Wu, J. Biol. Chem, 262: 4429-4432, 1987.

Zetter B R. Angiogenesis and tumor metastasis. Ann Rev Med 49:407-424, 1998

Zhang et al., J. Nature 372: 425, 1994.

Zhang J and Russell S. Vectors for cancer gene therapy Cancer Met. Rev. 3:385-401, 1996.

ZHANG, W. (1999). Development and application of adenoviral vectors for gene therapy of cancer. Cancer Gene Ther. 6; 113-138.

Zini, S., Fournie-Zaluski, M. C., Chauvel, E., Roques, B., Corvol, P. and Cortes-Llorens, C. (1996) Identification of metabolic pathways of brain angiotensin II and ]II using specific aminopeptidase inhibitors: predominant role of angiotensin III in the control of vasopressin release. Proc Natl Acad Sci USA, 93, 11968-11973.

What is claimed is:

1. A method of identifying targeting peptides comprising:
a) obtaining at least one sample from an organ, tissue or cell type of interest;
b) exposing said sample to a phage display library; and
c) recovering phage bound to said sample; wherein said phage express targeting peptides.

2. The method of claim 1, wherein said phage are recovered by infecting pilus positive bacteria.

3. The method of claim 1, wherein said phage are recovered by (i) amplifying phage inserts; (ii) ligating the amplified inserts to phage DNA; and (iii) producing phage from the ligated DNA.

4. The method of claim 1, wherein the sample is a thin section of an organ or tissue.

5. The method of claim 4, wherein bound phage are recovered by PALM (Positioning and Ablation with Laser Microbeams).

6. The method of claim 1, wherein bound phage are recovered by BRASIL (Biopanning and Rapid Analysis of Selective Interactive Ligands).

7. The method of claim 1, further comprising: (i) preselecting the phage library against a first organ, tissue or cell type; (ii) removing phage that bind to said first organ, tissue or cell type; and (iii) selecting the remaining phage against a second organ, tissue or cell type.

8. The method of claim 7, wherein said first cell type is quiescent and said second cell type has been stimulated.

9. The method of claim 8, wherein said second cell type has been stimulated with a hormone, growth factor, cytokine, chemokine, neurotransmitter, angiogenic agent, pro-apoptosis agent, anti-apoptosis agent, anti-angiogenic agent, phorbol ester, protein or peptide.

10. A method of detecting the presence of a target for a targeting peptide comprising:
a) obtaining a sample of a tissue, organ or cell type;
b) exposing the sample to phage expressing one or more targeting peptide sequences; and
c) detecting the presence of phage bound to said sample.

11. The method of claim 10, wherein the sample is a thin section or biopsy sample.

12. The method of claim 10, wherein the presence of the target indicates a disease.

13. The method of claim 10, wherein the presence of the target indicates that the tissue, organ or cell type will respond to a therapy.

14. The method of claim 13, wherein the therapy comprises administration of a targeting peptide attached to a therapeutic agent.

15. The method of claim 13, wherein the therapy is directed against the target.

16. The method of claim 10, wherein the target is a peptide, protein, glycoprotein, lipoprotein, epitope, lipid, carbohydrate, multi-molecular structure, a specific conformation of one or more molecules or a morphoanatomic entity

17. The method of claim 1, wherein the sample comprises antibodies from a subject with a disease.

18. The method of claim 17, wherein the sample is ascites, blood, serum, lymphatic fluid, spleen, tumor or lymph node tissue.

19. The method of claim 17, wherein the antibodies have been purified from the sample.

20. The method of claim 17, wherein the disease is cancer, metastatic cancer, Hodgkin's disease, ovarian cancer or prostate cancer.

21. The method of claim 20, further comprising: (i) obtaining at least one targeting peptide that binds to antibodies from a cancer subject; and (ii) producing a vaccine comprising the at least one peptide.

22. The method of claim 21, further comprising immunizing a subject with the vaccine.

23. The method of claim 21, further comprising activating T cells in vitro with the vaccine.

24. The method of claim 17, wherein the phage library is prescreened against antibodies from a normal subject.

25. The method of claim 17, further comprising purifying antibodies that bind to the targeting peptides.

26. An antibody purified by the method of claim 25.

27. A method comprising:
a) obtaining one or more antibodies according to claim 26; and
b) attaching the antibody to an agent.

28. The method of claim 27, wherein the agent is a drug, a chemotherapeutic agent, a radioisotope, a pro-apoptosis agent, an anti-angiogenic agent, an enzyme, a hormone, a cytokine, a growth factor, a cytotoxic agent, a peptide, a protein, an antibiotic, an antibody, a Fab fragment of an antibody, an imaging agent, an antigen, a survival factor, an anti-apoptotic agent, a hormone antagonist, a virus, a bacteriophage, a bacterium, a liposome, a microparticle, a magnetic bead, a microdevice, a yeast cell, a mammalian cell, a cell or an expression vector.

29. The method of claim 27, further comprising administering the antibody and agent to a subject.

30. The method of claim 27, further comprising reacting the antibody and agent with a tissue sample.

31. The method of claim 27, further comprising treating a subject with a disease.

32. The method of claim 27, further comprising obtaining an image of the subject.

33. The method of claim 29, further comprising detecting the presence of an antigen.

34. The method of claim 30, further comprising detecting the presence of an antigen.

35. A peptide prepared by the method of claim 1.

36. A method comprising:
a) obtaining one or more peptides according to claim 35; and
b) attaching the peptide to an agent.

37. The method of claim 36, wherein the agent is a drug, a chemotherapeutic agent, a radioisotope, a pro-apoptosis agent, an anti-angiogenic agent, an enzyme, a hormone, a cytokine, a growth factor, a cytotoxic agent, a peptide, a protein, an antibiotic, an antibody, a. Fab fragment of an antibody, an imaging agent, an antigen, a survival factor, an anti-apoptotic agent, a hormone antagonist, a virus, a bacteriophage, a bacterium, a liposome, a microparticle, a magnetic bead, a microdevice, a yeast cell, a mammalian cell, a cell or an expression vector.

38. The method of claim 37, further comprising administering the peptide and agent to a subject.

39. The method of claim 37, further comprising reacting the peptide and agent with a tissue sample.

40. The method of claim 38, further comprising treating a subject with a disease.

41. The method of claim 38, further comprising obtaining an image of the subject.

42. The method of claim 38, further comprising detecting the presence of a ligand or receptor for the peptide.

43. The method of claim 39, further comprising detecting the presence of a ligand or receptor for the peptide.

44. The method of claim 1, wherein the library expressess targeting peptides prepared from antibody sequences.

45. The method of claim 44, further comprising: (i) obtaining a sample of spleen from a subject; (ii) obtaining mRNA from the sample; (iii) amplifying antibody mRNA sequences to form double-stranded cDNAs encoding antibody peptides; and (iv) inserting the cDNAs into a phage display library.

46. The method of claim 45, further comprising immunizing the subject with at least one antigen before collecting the spleen sample.

47. A method for identifying an endogenous receptor or ligand comprising:
a) exposing a sample from an organ, tissue or cell type to a phage display library;
b) obtaining phage that bind to the sample;
c) sequencing the phage inserts; and
d) identifying an endogenous receptor or ligand homologous with at least one phage insert.

48. The method of claim 47, further comprising: (i) preparing antibodies against at least one phage insert; and (ii) using the antibodies to purify an endogenous receptor or ligand.

49. A method of identifying a virus associated with a disease state comprising:
a) pre-clearing a phage display library on immunoglobulins from a subject without the disease state;
b) screening the pre-cleared library on immunoglobulins from a subject with the disease state;
c) recovering phage that bind to immunoglobulins from said subject with the disease state;
d) determining the amino acid sequences of targeting peptides displayed on said collected phage; and
e) identifying one or more viruses encoding proteins homologous with the targeting peptides.

50. The method of claim 49, wherein the disease state is Hodgkin's disease.

51. A gene therapy vector comprising:
a) a bacteriophage, wherein the phage expresses a targeting peptide as part of a surface protein;
b) a gene encoding a therapeutic protein or an anti-sense RNA, wherein the gene is incorporated into the phage genome; and
c) cis-elements of an adeno-associated virus (AAV), wherein said elements are incorporated into the phage genome.

52. The gene therapy vector of claim 51, wherein the phage is an fUS5 phage.

53. The gene therapy vector of claim 52, wherein the targeting peptide is expressed as part of the PIII or PVIII coat proteins.

54. A method of treating a disease comprising:
a) obtaining a gene therapy vector according to claim 51; and
b) administering said vector to a subject with a disease.

55. The method of claim 54, wherein the targeting peptide binds to cells associated with the disease.

56. An isolated peptide of 100 amino acids or less in size, comprising at least 3 contiguous amino acids of a sequence selected from any of SEQ ID NO: 5 through SEQ ID NO: 65, SEQ ID NO: 67 through SEQ D NO: 165 and SEQ ID NO: 176 through SEQ ID NO: 270.

57. The isolated peptide of claim 56, wherein said peptide is 50 amino acids or less in size.

58. The isolated peptide of claim 56, wherein said peptide is 25 amino acids or less in size.

59. The isolated peptide of claim 56, wherein said peptide is 10 amino acids or less in size.

60. The isolated peptide of claim 56, wherein said peptide is 7 amino acids or less in size.

61. The isolated peptide of claim 56, wherein said peptide is 5 amino acids or less in size.

62. The isolated peptide of claim 56, wherein said peptide comprises at least 5 contiguous amino acids of a sequence selected from any of SEQ ID NO: 5 through SEQ ID NO: 65, SEQ ID NO: 67 through SEQ ID NO: 165 and SEQ ID NO: 176 through SEQ ID NO: 270.

63. The isolated peptide of claim 56, wherein said peptide is attached to a molecule.

64. A fusion protein, comprising at least 3 contiguous amino acids selected from any of SEQ ID NO: 5 through SEQ ID NO: 65, SEQ ID NO: 67 through SEQ ID NO: 165 and SEQ ID NO: 176 through SEQ ID NO: 272.

65. The isolated peptide of claim 63, wherein said attachment is covalent.

66. The isolated peptide of claim 63, wherein said molecule is a drug, a chemotherapeutic agent, a radioisotope, a pro-apoptosis agent, an anti-angiogenic agent, a hormone, a cytokine, a growth factor, a cytotoxic agent, a peptide, a protein, an antibiotic, an antibody, a Fab fragment of an antibody, an imaging agent, an antigen, a survival factor, an anti-apoptotic agent or a hormone antagonist.

67. The isolated peptide of claim 66, wherein said pro-aptoptosis agent is selected from the group consisting of gramicidin, magainin, mellitin, defensin, cecropin, (KLAKLAK)₂ (SEQ ID NO: 1), (KLAKKLA)₂ (SEQ ID NO: 2), (KAAKKAA)₂ (SEQ ID NO: 3) and (KLGKKLG)₃ (SEQ ID NO: 4).

68. The isolated peptide of claim 66, wherein said anti-angiogenic agent is selected from the group consisting of thrombospondin, angiostatin5, pigment epithelium-drived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, accutin, cidofovir, vincristine, bleomycin, AGM-1470, platelet factor 4 and minocycline.

69. The isolated peptide of claim 66, wherein said cytokine is selected from the group consisting of interleukin 1 (IL-1), IL,2, IL-5, I10, IL-11, IL,12, IL,18, interferon-γ (IF-γ), IF-α, IF-β, tumor necrosis factor-α (TNF-α), or GM-CSF (granulocyte macrophage colony stimulating factor).

70. The isolated peptide of claim 56, wherein said peptide is attached to a virus, a bacteriophage, a bacterium, a liposome, a microparticle, a magnetic bead, a microdevice, a yeast cell, a mammalian cell or a cell.

71. The isolated peptide of claim 56, wherein said peptide is attached to a eukaryotic expression vector.

72. The isolated peptide of claim 71, wherein said vector is a gene therapy vector.

73. The isolated peptide of claim 56, wherein said peptide is attached to a solid support.

74. A composition comprising the isolated peptide of claim 56 in a pharmaceutically acceptable carrier.

75. The composition of claim 74, wherein said peptide is attached to a molecule.

76. The composition of claim 74, wherein said peptide is attached to a macromolecular complex.

77. A kit comprising the isolated peptide of claim 56 and a control peptide, each in a container.

78. An antibody or antibody fragment that selectively binds to an isolated peptide, the peptide comprising at least three contigous amino acids selected from any of SEQ ID NO: 5 through SEQ ID NO: 65, SEQ ID NO: 67 through SEQ ID NO: 165 and SEQ ID NO: 176 through SEQ ID NO: 270.

79. A method of diagnosing prostate cancer comprising:
a) obtaining an antibody or antibody fragment that binds to an isolated peptide comprising at least three contiguous amino acids selected from any of SEQ ID NO: 58 through SEQ ID NO: 88;
b) contacting a sample from a subject with the antibody or antibody fragment; and
c) detecting binding of the antibody or antibody fragment to the sample.

80. The method of claim 79, wherein the antibody or antibody fragment binds to an isolated peptide of an amino acid sequence of SEQ ID NO: 60.

81. The method of claim 79, wherein the antibody or antibody fragment binds to an isolated peptide of an amino acid sequence of SEQ ID NO: 67.

82. The method of claim 79, wherein the antibody or antibody fragment binds to grp78 (glucose regulated protein of 78,000 molecular weight).

83. A method of treating prostate cancer comprising:
a) obtaining an antibody or antibody fragment that binds to an isolated peptide comprising at least three contiguous amino acids selected from any of SEQ ID NO: 58 through SEQ ID NO: 88;
b) contacting a sample from a subject with the antibody or antibody fragment; and
c) detecting binding of the antibody or antibody fragment to the sample;
d) preparing a vaccine from the isolated peptide; and
e) immunizing a subject with the vaccine.

84. The method of claim 83, further comprising stimulating immune system cells in vitro with the vaccine.

85. A method of diagnosing prostate cancer comprising:
a) obtaining an isolated protein or peptide of an amino acid sequence selected any of SEQ ID NO: 58 through SEQ ID NO: 88;
b) contacting the protein or peptide with an antibody-containing sample from a patient; and
c) detecting binding of an antibody to the protein or peptide.

86. The method of claim 85, further comprising determining the patient's likelihood of three year survival.

87. The method of claim 85, wherein the isolated peptide has the amino acid sequence of SEQ ID NO: 60.

88. The method of claim 85, wherein the isolated peptide has the amino acid sequence of SEQ ID NO: 67.

89. The method of claim 85, wherein the isolated protein is grp78.

90. The method of claim 85, wherein the sample comprises blood, serum, ascites, lymphatic fluid, spleen, lymph node or prostate tissue.

91. The method of claim 85, wherein the sample comprises antibodies, the antibodies obtained from a tumor, a tisue, a peluran effusion or spinal fluid.

92. A method of targeting delivery to a prostate cancer comprising:
a) obtaining an antibody or antibody fragment that binds to an isolated peptide comprising at least three contiguous amino acids selected from any of SEQ ID NO: 58 through SEQ ID NO: 88;
b) attaching the antibody or antibody fragment to a therapeutic agent to form a complex; and
c) administering the complex to a subject with prostate cancer.

93. An antibody or antibody fragment (Fab) that binds to an isolated protein or peptide of an amino acid sequence selected from the group consisting of SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164 and SEQ ID NO: 165.

94. A method of diagnosing Hodgkin's disease comprising:
a) obtaining an antibody or antibody fragment according to claim 93;
b) contacting a sample from a patient with the antibody or antibody fragment; and
c) detecting binding of the antibody or antibody fragment to the sample.

95. The method of claim 94, wherein the sample comprises blood, serum, ascites, lymphatic fluid, spleen or lymph node tissue.

96. A method of targeting delivery to a Hodgin's disease cell comprising:
a) obtaining an antibody or antibody fragment according to claim 93;
b) attaching the antibody or antibody fragment to a therapeutic agent to form a complex; and
c) administering the complex to a patient with Hodkin's disease.

97. A method of treating Hodgkin's disease comprising:
a) obtaining an antibody or antibody fragment that binds to an isolated peptide comprising at least three contiguous amino acids selected from SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164 or SEQ ID NO: 165;
b) contacting a sample from a subject with the antibody or antibody fragment; and
c) detecting binding of the antibody or antibody fragment to the sample;
d) preparing a vaccine from the isolated peptide; and
e) immunizing a subject with the vaccine or stimulating immune system cells in vitro with the vaccine.

98. A gene therapy vector, wherein the vector expresses a targeting peptide sequence as prt of a surface protein, the targeting peptide comprising at least three contiguous amino acids selected from any of SEQ ID NO: 5 through SEQ ID NO: 65, SEQ ID NO: 67 through SEQ ID NO: 165 and SEQ ID NO: 176 through SEQ ID NO: 272.

99. The vector of claim 98, wherein the vector is a chimeric phage comprising elements from adeno-associated virus.

100. The vector of claim 99, further comprising a gene encoding a therapeutic protein or peptide.

101. A targeting peptide against human antibodies from an individual with Hodgkin's disease comprising at least three contiguous amino acids selected from SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164 and SEQ ID NO: 165.

102. A method for identifying an endogenous homolog for the isolated peptide of claim 56, comprising:
a) obtaining an antibody to said peptide; and
b) identifying the homolog with the antibody.

103. The method of claim 102, further comprising immunopurifying the homolog with the antibody.

104. An isolated nucleic acid of 300 nucleotides or less in size, said nucleic acid encoding a peptide according to claim 56.

105. A vector comprising the isolated nucleic acid of claim 104.

106. The vector of claim 105, wherein said vector is an expression vector.

107. The vector of claim 106, wherein said vector is a prokaryotic or eukaryotic expression vector.

108. The vector of claim 107, wherein said vector is a plasmid, a cosmid, a yeast artificial chromosome (YAC), a bacterial artificial chromosome SAC), a virus or a phage.

109. A method of treating a disease state comprising:
a) selecting a peptide according to claim 56, wherein said peptide is targeted to cells associated with said disease state;
b) attaching to said peptide one or more molecules effective to treat said disease state; and
c) administering said peptide to a subject with said disease state.

110. The method of claim 109, wherein said disease state is selected from the group consisting of diabetes, inflammatory disease, arthritis, atherosclerosis, cancer, autoimmune disease, bacterial infection, viral infection, cardiovascular disease or degenerative disease.

111. A method of identifying targeting peptides against antibodies from an individual with a disease state comprising:
a) obtaining a sample from said individual;
b) obtaining antibodies from said sample;
c) adding a phage display library to said antibodies; and
d) collecting phage bound to said antibodies.

112. The method of claim 111, further comprising coupling said antibodies to a solid support prior to adding the phage display library.

113. The method of claim 112, wherein said solid support comprises protein G attached to beads.

114. The method of claim 113, further comprising:
a) adding said phage library to antibodies obtained from an individual who does not have said disease state;
b) removing phage binding to said antibodies from said library; and
c) adding the remaining phage to antibodies from an individual with said disease state.

115. A method of obtaining antibodies against one or more targeting peptides comprising:
a) preparing one or more targeting peptides according to claim 52;
b) immobilizing said one or more peptides on a solid support;
c) exposing said peptides to a sample containing antibodies; and
d) collecting antibodies that bind to said one or more peptides.

116. A method of blocking metastasis comprising:
a) screening a phage library against an ascites sample from an individual with cancer;
b) obtaining at least one targeting peptide that binds to an antibody from said sample; and
c) administering said peptide to a patient with cancer.

117. The method of claim 116, wherein the peptide is administered into the peritoneal cavity of the patient.

118. The method of claim 117, wherein the cancer is ovarian cancer, primary peritoneal cancer, mesothelioma, Mullerian cancer, liver cancer or gastrointestinal cancer.


SEQUENCE LISTING (273 sequences)

SEQ ID NO: 1 - protein, 14 aa, Artificial Sequence
  Peptide (1)..(14): synthetic construct
KLAKLAKKLAKLAK

SEQ ID NO: 2 - protein, 14 aa, Artificial Sequence
  Peptide (1)..(14): synthetic construct
KLAKKLAKLAKKLA

SEQ ID NO: 3 - protein, 14 aa, Artificial Sequence
  Peptide (1)..(14): synthetic construct
KAAKKAAKAAKKAA

SEQ ID NO: 4 - protein, 21 aa, Artificial Sequence
  Peptide (1)..(21): synthetic construct
KLGKKLGKLGKKLGKLGKKLG

SEQ ID NO: 5 - protein, 13 aa, Artificial Sequence
  Peptide (1)..(13): synthetic construct
CGLRCPLVCPGGC

SEQ ID NO: 6 - protein, 13 aa, Artificial Sequence
  Peptide (1)..(13): synthetic construct
CPVGCGGGCRPAC

SEQ ID NO: 7 - protein, 13 aa, Artificial Sequence
  Peptide (1)..(13): synthetic construct
CEVLCGVDCSNRC

SEQ ID NO: 8 - protein, 7 aa, Artificial Sequence
  Peptide (1)..(7): synthetic construct
WVSPVLG

SEQ ID NO: 9 - protein, 7 aa, Artificial Sequence
  Peptide (1)..(7): synthetic construct
VLGPRAM

SEQ ID NO: 10 - protein, 7 aa, Artificial Sequence
  Peptide (1)..(7): synthetic construct
LVGKWPY

SEQ ID NO: 11 - protein, 7 aa, Artificial Sequence
  Peptide (1)..(7): synthetic construct
PSRRLGS

SEQ ID NO: 12 - protein, 7 aa, Artificial Sequence
  Peptide (1)..(7): synthetic construct
APNTPVL

SEQ ID NO: 13 - protein, 9 aa, Artificial Sequence
  Peptide (1)..(9): synthetic construct
CRLADKELC

SEQ ID NO: 14 - protein, 9 aa, Artificial Sequence
  Peptide (1)..(9): synthetic construct
CRLSLPELC

SEQ ID NO: 15 - protein, 6 aa, Artificial Sequence
  Peptide (1)..(6): synthetic construct
TSRFSL

SEQ ID NO: 16 - protein, 7 aa, Artificial Sequence
  Peptide (1)..(7): synthetic construct
SLHRVAR

SEQ ID NO: 17 - protein, 7 aa, Artificial Sequence
  Peptide (1)..(7): synthetic construct
PLLVRTV

SEQ ID NO: 18 - protein, 7 aa, Artificial Sequence
  Peptide (1)..(7): synthetic construct
GRGRMTS

SEQ ID NO: 19 - protein, 9 aa, Artificial Sequence
  Peptide (1)..(9): synthetic construct
CSSPFHDSC

SEQ ID NO: 20 - protein, 9 aa, Artificial Sequence
  Peptide (1)..(9): synthetic construct
CTSAASGLC

SEQ ID NO: 21 - protein, 9 aa, Artificial Sequence
  Peptide (1)..(9): synthetic construct
CPGPLNPPC

SEQ ID NO: 22 - protein, 9 aa, Artificial Sequence
  Peptide (1)..(9): synthetic construct
CAFNNDDVC

SEQ ID NO: 23 - protein, 9 aa, Artificial Sequence
  Peptide (1)..(9): synthetic construct
CQISIWRTC

SEQ ID NO: 24 - protein, 9 aa, Artificial Sequence
  Peptide (1)..(9): synthetic construct
CSPPLTRWC

SEQ ID NO: 25 - protein, 9 aa, Artificial Sequence
  Peptide (1)..(9): synthetic construct
CQSDCIDLC

SEQ ID NO: 26 - protein, 13 aa, Artificial Sequence
  Peptide (1)..(13): synthetic construct
CGAGCGVPCPGGC

SEQ ID NO: 27 - protein, 3 aa, Artificial Sequence
  Peptide (1)..(3): synthetic construct
FHD

SEQ ID NO: 28 - protein, 4 aa, Artificial Sequence
  Peptide (1)..(4): synthetic construct
FHDS

SEQ ID NO: 29 - protein, 3 aa, Artificial Sequence
  Peptide (1)..(3): synthetic construct
HDS

SEQ ID NO: 30 - protein, 3 aa, Artificial Sequence
  Peptide (1)..(3): synthetic construct
PFH

SEQ ID NO: 31 - protein, 4 aa, Artificial Sequence
  Peptide (1)..(4): synthetic construct
PFHD

SEQ ID NO: 32 - protein, 5 aa, Artificial Sequence
  Peptide (1)..(5): synthetic construct
PFHDS

SEQ ID NO: 33 - protein, 3 aa, Artificial Sequence
  Peptide (1)..(3): synthetic construct
SPF

SEQ ID NO: 34 - protein, 4 aa, Artificial Sequence
  Peptide (1)..(4): synthetic construct
SPFH

SEQ ID NO: 35 - protein, 5 aa, Artificial Sequence
  Peptide (1)..(5): synthetic construct
SPFHD

SEQ ID NO: 36 - protein, 6 aa, Artificial Sequence
  Peptide (1)..(6): synthetic construct
SPFHDS

SEQ ID NO: 37 - protein, 3 aa, Artificial Sequence
  Peptide (1)..(3): synthetic construct
SSP

SEQ ID NO: 38 - protein, 4 aa, Artificial Sequence
  Peptide (1)..(4): synthetic construct
SSPF

SEQ ID NO: 39 - protein, 5 aa, Artificial Sequence
  Peptide (1)..(5): synthetic construct
SSPFH

SEQ ID NO: 40 - protein, 6 aa, Artificial Sequence
  Peptide (1)..(6): synthetic construct
SSPFHD

SEQ ID NO: 41 - protein, 7 aa, Artificial Sequence
  Peptide (1)..(7): synthetic construct
SSPFHDS

SEQ ID NO: 42 - protein, 3 aa, Artificial Sequence
  Peptide (1)..(3): synthetic construct
SSF

SEQ ID NO: 43 - protein, 3 aa, Artificial Sequence
  Peptide (1)..(3): synthetic construct
SSS

SEQ ID NO: 44 - protein, 3 aa, Artificial Sequence
  Peptide (1)..(3): synthetic construct
FSV

SEQ ID NO: 45 - protein, 4 aa, Artificial Sequence
  Peptide (1)..(4): synthetic construct
FSVT

SEQ ID NO: 46 - protein, 3 aa, Artificial Sequence
  Peptide (1)..(3): synthetic construct
SAA

SEQ ID NO: 47 - protein, 3 aa, Artificial Sequence
  Peptide (1)..(3): synthetic construct
SFS

SEQ ID NO: 48 - protein, 4 aa, Artificial Sequence
  Peptide (1)..(4): synthetic construct
SFSV

SEQ ID NO: 49 - protein, 5 aa, Artificial Sequence
  Peptide (1)..(5): synthetic construct
SFSVT

SEQ ID NO: 50 - protein, 4 aa, Artificial Sequence
  Peptide (1)..(4): synthetic construct
SSFS

SEQ ID NO: 51 - protein, 5 aa, Artificial Sequence
  Peptide (1)..(5): synthetic construct
SSFSV

SEQ ID NO: 52 - protein, 4 aa, Artificial Sequence
  Peptide (1)..(4): synthetic construct
SSSF

SEQ ID NO: 53 - protein, 5 aa, Artificial Sequence
  Peptide (1)..(5): synthetic construct
SSSFS

SEQ ID NO: 54 - protein, 6 aa, Artificial Sequence
  Peptide (1)..(6): synthetic construct
SSSFSV

SEQ ID NO: 55 - protein, 7 aa, Artificial Sequence
  Peptide (1)..(7): synthetic construct
SSSFSVT

SEQ ID NO: 56 - protein, 6 aa, Artificial Sequence
  Peptide (1)..(6): synthetic construct
SSFSVT

SEQ ID NO: 57 - protein, 3 aa, Artificial Sequence
  Peptide (1)..(3): synthetic construct
SVT

SEQ ID NO: 58 - protein, 8 aa, Artificial Sequence
  Peptide (1)..(8): synthetic construct
CHQKPWEC

SEQ ID NO: 59 - protein, 8 aa, Artificial Sequence
  Peptide (1)..(8): synthetic construct
CKDRFERC

SEQ ID NO: 60 - protein, 8 aa, Artificial Sequence
  Peptide (1)..(8): synthetic construct
CNVSDKSC

SEQ ID NO: 61 - protein, 8 aa, Artificial Sequence
  misc_feature (3)..(3): Unidentified amino acid
CNXSDKSC

SEQ ID NO: 62 - protein, 8 aa, Artificial Sequence
  misc_feature (3)..(3): Unidentified amino acid
CNXTDKSC

SEQ ID NO: 63 - protein, 8 aa, Artificial Sequence
  Peptide (1)..(8): synthetic construct
CNWTDKTC

SEQ ID NO: 64 - protein, 8 aa, Artificial Sequence
  Peptide (1)..(8): synthetic construct
CNITQKSC

SEQ ID NO: 65 - protein, 8 aa, Artificial Sequence
  Peptide (1)..(8): synthetic construct
CNKTDKGC

SEQ ID NO: 66 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 67 - protein, 8 aa, Artificial Sequence
  Peptide (1)..(8): synthetic construct
CTFAGSSC

SEQ ID NO: 68 - protein, 8 aa, Artificial Sequence
  Peptide (1)..(8): synthetic construct
CNSAFAGC

SEQ ID NO: 69 - protein, 8 aa, Artificial Sequence
  Peptide (1)..(8): synthetic construct
CSYTFAGC

SEQ ID NO: 70 - protein, 8 aa, Artificial Sequence
  Peptide (1)..(8): synthetic construct
CSTFAGSC

SEQ ID NO: 71 - protein, 8 aa, Artificial Sequence
  Peptide (1)..(8): synthetic construct
CRDGYHHC

SEQ ID NO: 72 - protein, 8 aa, Artificial Sequence
  Peptide (1)..(8): synthetic construct
CSASDLSC

SEQ ID NO: 73 - protein, 8 aa, Artificial Sequence
  Peptide (1)..(8): synthetic construct
CQNQYPEC

SEQ ID NO: 74 - protein, 8 aa, Artificial Sequence
  Peptide (1)..(8): synthetic construct
CRASAMVC

SEQ ID NO: 75 - protein, 8 aa, Artificial Sequence
  Peptide (1)..(8): synthetic construct
CIDMTHQC

SEQ ID NO: 76 - protein, 8 aa, Artificial Sequence
  Peptide (1)..(8): synthetic construct
CISSPSNC

SEQ ID NO: 77 - protein, 8 aa, Artificial Sequence
  Peptide (1)..(8): synthetic construct
CNQSMWSC

SEQ ID NO: 78 - protein, 8 aa, Artificial Sequence
  Peptide (1)..(8): synthetic construct
CQFENGTC

SEQ ID NO: 79 - protein, 8 aa, Artificial Sequence
  Peptide (1)..(8): synthetic construct
CAVKSVTC

SEQ ID NO: 80 - protein, 8 aa, Artificial Sequence
  Peptide (1)..(8): synthetic construct
CNGFMGYC

SEQ ID NO: 81 - protein, 8 aa, Artificial Sequence
  Peptide (1)..(8): synthetic construct
CLTSENAC

SEQ ID NO: 82 - protein, 8 aa, Artificial Sequence
  Peptide (1)..(8): synthetic construct
CRASAMVC

SEQ ID NO: 83 - protein, 8 aa, Artificial Sequence
  Peptide (1)..(8): synthetic construct
CSKKFVTC

SEQ ID NO: 84 - protein, 8 aa, Artificial Sequence
  Peptide (1)..(8): synthetic construct
CKNKHTTC

SEQ ID NO: 85 - protein, 8 aa, Artificial Sequence
  Peptide (1)..(8): synthetic construct
CFETFAGC

SEQ ID NO: 86 - protein, 8 aa, Artificial Sequence
  Peptide (1)..(8): synthetic construct
CNNMYAGC

SEQ ID NO: 87 - protein, 8 aa, Artificial Sequence
  Peptide (1)..(8): synthetic construct
CFPKRVTC

SEQ ID NO: 88 - protein, 8 aa, Artificial Sequence
  Peptide (1)..(8): synthetic construct
CPRSAKNC

SEQ ID NO: 89 - protein, 8 aa, Artificial Sequence
  Peptide (1)..(8): synthetic construct
CAGYAVDC

SEQ ID NO: 90 - protein, 8 aa, Artificial Sequence
  Peptide (1)..(8): synthetic construct
CAMGSPEC

SEQ ID NO: 91 - protein, 8 aa, Artificial Sequence
  Peptide (1)..(8): synthetic construct
CEAGRGGC

SEQ ID NO: 92 - protein, 8 aa, Artificial Sequence
  Peptide (1)..(8): synthetic construct
CKLSGTRC

SEQ ID NO: 93 - protein, 8 aa, Artificial Sequence
  Peptide (1)..(8): synthetic construct
CNGIVQVC

SEQ ID NO: 94 - protein, 8 aa, Artificial Sequence
  Peptide (1)..(8): synthetic construct
CASSHAVC

SEQ ID NO: 95 - protein, 8 aa, Artificial Sequence
  Peptide (1)..(8): synthetic construct
CWQGSVSC

SEQ ID NO: 96 - protein, 8 aa, Artificial Sequence
  Peptide (1)..(8): synthetic construct
CMVGYIVC

SEQ ID NO: 97 - protein, 8 aa, Artificial Sequence
  Peptide (1)..(8): synthetic construct
CWNRGSTC

SEQ ID NO: 98 - protein, 8 aa, Artificial Sequence
  Peptide (1)..(8): synthetic construct
CPERGTRC

SEQ ID NO: 99 - protein, 8 aa, Artificial Sequence
  Peptide (1)..(8): synthetic construct
CVNKYIPC

SEQ ID NO: 100 - protein, 8 aa, Artificial Sequence
  Peptide (1)..(8): synthetic construct
CGTAEGVC

SEQ ID NO: 101 - protein, 8 aa, Artificial Sequence
  Peptide (1)..(8): synthetic construct
CASPNLAC

SEQ ID NO: 102 - protein, 8 aa, Artificial Sequence
  Peptide (1)..(8): synthetic construct
CDNGNASC

SEQ ID NO: 103 - protein, 8 aa, Artificial Sequence
  Peptide (1)..(8): synthetic construct
CSQLKLGC

SEQ ID NO: 104 - protein, 8 aa, Artificial Sequence
  Peptide (1)..(8): synthetic construct
CMGTKSSC

SEQ ID NO: 105 - protein, 8 aa, Artificial Sequence
  Peptide (1)..(8): synthetic construct
CIDTSELC

SEQ ID NO: 106 - protein, 8 aa, Artificial Sequence
  Peptide (1)..(8): synthetic construct
CGRVPQMC

SEQ ID NO: 107 - protein, 8 aa, Artificial Sequence
  Peptide (1)..(8): synthetic construct
CAGFSSPC

SEQ ID NO: 108 - protein, 8 aa, Artificial Sequence
  Peptide (1)..(8): synthetic construct
CSRSSFLC

SEQ ID NO: 109 - protein, 8 aa, Artificial Sequence
  Peptide (1)..(8): synthetic construct
CIRPNDHC

SEQ ID NO: 110 - protein, 8 aa, Artificial Sequence
  Peptide (1)..(8): synthetic construct
CVSRPRAC

SEQ ID NO: 111 - protein, 8 aa, Artificial Sequence
  Peptide (1)..(8): synthetic construct
CMGQGQAC

SEQ ID NO: 112 - protein, 8 aa, Artificial Sequence
  Peptide (1)..(8): synthetic construct
CADMQGTC

SEQ ID NO: 113 - protein, 8 aa, Artificial Sequence
  Peptide (1)..(8): synthetic construct
CAIHDSEC

SEQ ID NO: 114 - protein, 8 aa, Artificial Sequence
  Peptide (1)..(8): synthetic construct
CLVGAVQC

SEQ ID NO: 115 - protein, 9 aa, Artificial Sequence
  Peptide (1)..(9): synthetic construct
CAYTNSLRC

SEQ ID NO: 116 - protein, 9 aa, Artificial Sequence
  Peptide (1)..(9): synthetic construct
CFHMPPNTC

SEQ ID NO: 117 - protein, 9 aa, Artificial Sequence
  Peptide (1)..(9): synthetic construct
CLTLKSNVC

SEQ ID NO: 118 - protein, 9 aa, Artificial Sequence
  Peptide (1)..(9): synthetic construct
CLSKLHYVC

SEQ ID NO: 119 - protein, 9 aa, Artificial Sequence
  Peptide (1)..(9): synthetic construct
CMESSSGLC

SEQ ID NO: 120 - protein, 9 aa, Artificial Sequence
  Peptide (1)..(9): synthetic construct
CNRMTSYSC

SEQ ID NO: 121 - protein, 9 aa, Artificial Sequence
  Peptide (1)..(9): synthetic construct
CPKDSSKMC

SEQ ID NO: 122 - protein, 9 aa, Artificial Sequence
  Peptide (1)..(9): synthetic construct
CKAFQRHHC

SEQ ID NO: 123 - protein, 9 aa, Artificial Sequence
  Peptide (1)..(9): synthetic construct
CCRLQVSHC

SEQ ID NO: 124 - protein, 9 aa, Artificial Sequence
  Peptide (1)..(9): synthetic construct
CYSDRNMDC

SEQ ID NO: 125 - protein, 9 aa, Artificial Sequence
  Peptide (1)..(9): synthetic construct
CYKPVHSPC

SEQ ID NO: 126 - protein, 9 aa, Artificial Sequence
  Peptide (1)..(9): synthetic construct
CCETITKDC

SEQ ID NO: 127 - protein, 9 aa, Artificial Sequence
  Peptide (1)..(9): synthetic construct
CLKYEQRPC

SEQ ID NO: 128 - protein, 9 aa, Artificial Sequence
  Peptide (1)..(9): synthetic construct
CPYDALASC

SEQ ID NO: 129 - protein, 9 aa, Artificial Sequence
  Peptide (1)..(9): synthetic construct
CRMMALHEC

SEQ ID NO: 130 - protein, 9 aa, Artificial Sequence
  Peptide (1)..(9): synthetic construct
CIVGAPRLC

SEQ ID NO: 131 - protein, 9 aa, Artificial Sequence
  Peptide (1)..(9): synthetic construct
CRSSGFGTC

SEQ ID NO: 132 - protein, 9 aa, Artificial Sequence
  Peptide (1)..(9): synthetic construct
CRHQSASAC

SEQ ID NO: 133 - protein, 9 aa, Artificial Sequence
  Peptide (1)..(9): synthetic construct
CDGVLMFGC

SEQ ID NO: 134 - protein, 9 aa, Artificial Sequence
  Peptide (1)..(9): synthetic construct
CYAAGNVFC

SEQ ID NO: 135 - protein, 8 aa, Artificial Sequence
  Peptide (1)..(8): synthetic construct
CASSHAVC

SEQ ID NO: 136 - protein, 9 aa, Artificial Sequence
  Peptide (1)..(9): synthetic construct
CNFAGPASC

SEQ ID NO: 137 - protein, 9 aa, Artificial Sequence
  Peptide (1)..(9): synthetic construct
CRSLPPVRC

SEQ ID NO: 138 - protein, 9 aa, Artificial Sequence
  Peptide (1)..(9): synthetic construct
CNPHKAQSC

SEQ ID NO: 139 - protein, 9 aa, Artificial Sequence
  Peptide (1)..(9): synthetic construct
CRRDTYGRC

SEQ ID NO: 140 - protein, 9 aa, Artificial Sequence
  Peptide (1)..(9): synthetic construct
CALPGGQIC

SEQ ID NO: 141 - protein, 9 aa, Artificial Sequence
  misc_feature (6)..(7): Unidentified amino acids
CWSLEXXKC

SEQ ID NO: 142 - protein, 9 aa, Artificial Sequence
  Peptide (1)..(9): synthetic construct
CWSAEWTKC

SEQ ID NO: 143 - protein, 9 aa, Artificial Sequence
  Peptide (1)..(9): synthetic construct
CWSLELSKC

SEQ ID NO: 144 - protein, 9 aa, Artificial Sequence
  Peptide (1)..(9): synthetic construct
CWSLEFTKC

SEQ ID NO: 145 - protein, 9 aa, Artificial Sequence
  Peptide (1)..(9): synthetic construct
CWSLEVAKC

SEQ ID NO: 146 - protein, 9 aa, Artificial Sequence
  Peptide (1)..(9): synthetic construct
CWSLESLKC

SEQ ID NO: 147 - protein, 9 aa, Artificial Sequence
  Peptide (1)..(9): synthetic construct
CWSLELLKC

SEQ ID NO: 148 - protein, 9 aa, Artificial Sequence
  Peptide (1)..(9): synthetic construct
CGVVSRVGC

SEQ ID NO: 149 - protein, 9 aa, Artificial Sequence
  Peptide (1)..(9): synthetic construct
CLAWVGGRC

SEQ ID NO: 150 - protein, 9 aa, Artificial Sequence
  Peptide (1)..(9): synthetic construct
CQGKFSQRC

SEQ ID NO: 151 - protein, 9 aa, Artificial Sequence
  Peptide (1)..(9): synthetic construct
CRVLADRDC

SEQ ID NO: 152 - protein, 9 aa, Artificial Sequence
  Peptide (1)..(9): synthetic construct
CRWSSMIWC

SEQ ID NO: 153 - protein, 9 aa, Artificial Sequence
  Peptide (1)..(9): synthetic construct
CSLQGIVGC

SEQ ID NO: 154 - protein, 9 aa, Artificial Sequence
  Peptide (1)..(9): synthetic construct
CSRSVSRLC

SEQ ID NO: 155 - protein, 9 aa, Artificial Sequence
  Peptide (1)..(9): synthetic construct
CVAADRYVC

SEQ ID NO: 156 - protein, 9 aa, Artificial Sequence
  Peptide (1)..(9): synthetic construct
CYGAWRVSC

SEQ ID NO: 157 - protein, 9 aa, Artificial Sequence
  Peptide (1)..(9): synthetic construct
CSLLPASSC

SEQ ID NO: 158 - protein, 9 aa, Artificial Sequence
  Peptide (1)..(9): synthetic construct
CIGKGTSLC

SEQ ID NO: 159 - protein, 9 aa, Artificial Sequence
  Peptide (1)..(9): synthetic construct
CYVNVQVSC

SEQ ID NO: 160 - protein, 9 aa, Artificial Sequence
  Peptide (1)..(9): synthetic construct
CLGDIVERC

SEQ ID NO: 161 - protein, 9 aa, Artificial Sequence
  Peptide (1)..(9): synthetic construct
CMLVKRKNC

SEQ ID NO: 162 - protein, 9 aa, Artificial Sequence
  Peptide (1)..(9): synthetic construct
CAHFIINSC

SEQ ID NO: 163 - protein, 9 aa, Artificial Sequence
  Peptide (1)..(9): synthetic construct
CYYPGEKSC

SEQ ID NO: 164 - protein, 9 aa, Artificial Sequence
  Peptide (1)..(9): synthetic construct
CFSSFFRCC

SEQ ID NO: 165 - protein, 9 aa, Artificial Sequence
  Peptide (1)..(9): synthetic construct
CGIRGPNKC

SEQ ID NO: 166 - protein, 9 aa, Artificial Sequence
  Peptide (1)..(9): synthetic construct
CDCRGDCFC

SEQ ID NO: 167 - protein, 10 aa, Artificial Sequence
  Peptide (1)..(10): synthetic construct
CTTHWGFTLC

SEQ ID NO: 168 - protein, 13 aa, Artificial Sequence
  Peptide (1)..(13): synthetic construct
CGFECVRQCPERC

SEQ ID NO: 169 - protein, 5 aa, Artificial Sequence
  Peptide (1)..(5): synthetic construct
CARAC

SEQ ID NO: 170 - DNA, 43 bp, Artificial Sequence
  misc_feature (1)..(43): oligonucleotide
taatacgactcactatagggcaagctgataaaccgatacaatt

SEQ ID NO: 171 - DNA, 24 bp, Artificial Sequence
  misc_feature (1)..(24): oligonucleotide
ccctcatagttagcgtaacgatct

SEQ ID NO: 172 - DNA, 22 bp, Artificial Sequence
  misc_feature (1)..(22): oligonucleotide
cctttctattctcactcggccg

SEQ ID NO: 173 - DNA, 44 bp, Artificial Sequence
  misc_feature (1)..(44): oligonucleotide
caggaaacagctatgaccgctaaacaactttcaacagtttcggc

SEQ ID NO: 174 - DNA, 17 bp, Artificial Sequence
  misc_feature (1)..(17): oligonucleotide
cactcggccgacggggc

SEQ ID NO: 175 - DNA, 21 bp, Artificial Sequence
  misc_feature (1)..(21): oligonucleotide
cagtttcggccccagcggccc

SEQ ID NO: 176 - protein, 3 aa, Artificial Sequence
  Peptide (1)..(3): synthetic construct
GGL

SEQ ID NO: 177 - protein, 3 aa, Artificial Sequence
  Peptide (1)..(3): synthetic construct
LGG

SEQ ID NO: 178 - protein, 3 aa, Artificial Sequence
  Peptide (1)..(3): synthetic construct
VRG

SEQ ID NO: 179 - protein, 3 aa, Artificial Sequence
  Peptide (1)..(3): synthetic construct
AGG

SEQ ID NO: 180 - protein, 3 aa, Artificial Sequence
  Peptide (1)..(3): synthetic construct
VVG

SEQ ID NO: 181 - protein, 3 aa, Artificial Sequence
  Peptide (1)..(3): synthetic construct
VGG

SEQ ID NO: 182 - protein, 3 aa, Artificial Sequence
  Peptide (1)..(3): synthetic construct
GGL

SEQ ID NO: 183 - protein, 3 aa, Artificial Sequence
  Peptide (1)..(3): synthetic construct
GRV

SEQ ID NO: 184 - protein, 3 aa, Artificial Sequence
  Peptide (1)..(3): synthetic construct
GGA

SEQ ID NO: 185 - protein, 3 aa, Artificial Sequence
  Peptide (1)..(3): synthetic construct
GVV

SEQ ID NO: 186 - protein, 3 aa, Artificial Sequence
  Peptide (1)..(3): synthetic construct
GGV

SEQ ID NO: 187 - protein, 4 aa, Artificial Sequence
  Peptide (1)..(4): synthetic construct
GMWG

SEQ ID NO: 188 - protein, 3 aa, Artificial Sequence
  Peptide (1)..(3): synthetic construct
ALR

SEQ ID NO: 189 - protein, 3 aa, Artificial Sequence
  Peptide (1)..(3): synthetic construct
ALV

SEQ ID NO: 190 - protein, 4 aa, Artificial Sequence
  Peptide (1)..(4): synthetic construct
GGVH

SEQ ID NO: 191 - protein, 3 aa, Artificial Sequence
  Peptide (1)..(3): synthetic construct
VSG

SEQ ID NO: 192 - protein, 3 aa, Artificial Sequence
  Peptide (1)..(3): synthetic construct
GPW

SEQ ID NO: 193 - protein, 9 aa, Artificial Sequence
  Peptide (1)..(9): synthetic construct
CVPGLGGLC

SEQ ID NO: 194 - protein, 9 aa, Artificial Sequence
  Peptide (1)..(9): synthetic construct
CGGLDVRMC

SEQ ID NO: 195 - protein, 9 aa, Artificial Sequence
  Peptide (1)..(9): synthetic construct
CDGGLDWVC

SEQ ID NO: 196 - protein, 9 aa, Artificial Sequence
  Peptide (1)..(9): synthetic construct
CTWLGGREC

SEQ ID NO: 197 - protein, 9 aa, Artificial Sequence
  Peptide (1)..(9): synthetic construct
CSRWGLGGC

SEQ ID NO: 198 - protein, 9 aa, Artificial Sequence
  Peptide (1)..(9): synthetic construct
CPPLGGSRC

SEQ ID NO: 199 - protein, 9 aa, Artificial Sequence
  Peptide (1)..(9): synthetic construct
CVGGVRGGC

SEQ ID NO: 200 - protein, 9 aa, Artificial Sequence
  Peptide (1)..(9): synthetic construct
CVGNDVRGC

SEQ ID NO: 201 - protein, 9 aa, Artificial Sequence
  Peptide (1)..(9): synthetic construct
CESRLVRGC

SEQ ID NO: 202 - protein, 9 aa, Artificial Sequence
  Peptide (1)..(9): synthetic construct
CGGRPVRGC

SEQ ID NO: 203 - protein, 9 aa, Artificial Sequence
  Peptide (1)..(9): synthetic construct
CTPFIAGGC

SEQ ID NO: 204 - protein, 9 aa, Artificial Sequence
  Peptide (1)..(9): synthetic construct
CREWMAGGC

SEQ ID NO: 205 - protein, 9 aa, Artificial Sequence
  Peptide (1)..(9): synthetic construct
CAGGSLRVC

SEQ ID NO: 206 - protein, 9 aa, Artificial Sequence
  Peptide (1)..(9): synthetic construct
CEGVVGIVC

SEQ ID NO: 207 - protein, 9 aa, Artificial Sequence
  Peptide (1)..(9): synthetic construct
CDSVVGAWC

SEQ ID NO: 208 - protein, 9 aa, Artificial Sequence
  Peptide (1)..(9): synthetic construct
CRTAVVGSC

SEQ ID NO: 209 - protein, 9 aa, Artificial Sequence
  Peptide (1)..(9): synthetic construct
CVGGARALC

SEQ ID NO: 210 - protein, 9 aa, Artificial Sequence
  Peptide (1)..(9): synthetic construct
CLAHRVGGC

SEQ ID NO: 211 - protein, 9 aa, Artificial Sequence
  Peptide (1)..(9): synthetic construct
CWALSGGLC

SEQ ID NO: 212 - protein, 9 aa, Artificial Sequence
  Peptide (1)..(9): synthetic construct
CGGLVAYGC

SEQ ID NO: 213 - protein, 9 aa, Artificial Sequence
  Peptide (1)..(9): synthetic construct
CGGLATTTC

SEQ ID NO: 214 - protein, 9 aa, Artificial Sequence
  Peptide (1)..(9): synthetic construct
CGRVNSVAC

SEQ ID NO: 215 - protein, 9 aa, Artificial Sequence
  Peptide (1)..(9): synthetic construct
CAGRVALRC

SEQ ID NO: 216 - protein, 9 aa, Artificial Sequence
  Peptide (1)..(9): synthetic construct
CWNGGARAC

SEQ ID NO: 217 - protein, 9 aa, Artificial Sequence
  Peptide (1)..(9): synthetic construct
CLDRGGAHC

SEQ ID NO: 218 - protein, 9 aa, Artificial Sequence
  Peptide (1)..(9): synthetic construct
CELRGVVVC

SEQ ID NO: 219 - protein, 9 aa, Artificial Sequence
  Peptide (1)..(9): synthetic construct
CIGGVHYAC

SEQ ID NO: 220 - protein, 9 aa, Artificial Sequence
  Peptide (1)..(9): synthetic construct
CGGVHALRC

SEQ ID NO: 221 - protein, 9 aa, Artificial Sequence
  Peptide (1)..(9): synthetic construct
CIREGMWGC

SEQ ID NO: 222 - protein, 9 aa, Artificial Sequence
  Peptide (1)..(9): synthetic construct
CIRKGMWGC

SEQ ID NO: 223 - protein, 9 aa, Artificial Sequence
  Peptide (1)..(9): synthetic construct
CEALRLRAC

SEQ ID NO: 224 - protein, 9 aa, Artificial Sequence
  Peptide (1)..(9): synthetic construct
CALVNVHLC

SEQ ID NO: 225 - protein, 9 aa, Artificial Sequence
  Peptide (1)..(9): synthetic construct
CALVMVGAC

SEQ ID NO: 226 - protein, 9 aa, Artificial Sequence
  Peptide (1)..(9): synthetic construct
CMVSGVLLC

SEQ ID NO: 227 - protein, 9 aa, Artificial Sequence
  Peptide (1)..(9): synthetic construct
CGLVSGPWC

SEQ ID NO: 228 - protein, 9 aa, Artificial Sequence
  Peptide (1)..(9): synthetic construct
CLYDVSGGC

SEQ ID NO: 229 - protein, 9 aa, Artificial Sequence
  Peptide (1)..(9): synthetic construct
CSKVGPWWC

SEQ ID NO: 230 - protein, 9 aa, Artificial Sequence
  Peptide (1)..(9): synthetic construct
CGLVSGPWC

SEQ ID NO: 231 - protein, 9 aa, Artificial Sequence
  Peptide (1)..(9): synthetic construct
CAHHALMEC

SEQ ID NO: 232 - protein, 9 aa, Artificial Sequence
  Peptide (1)..(9): synthetic construct
CERPPFLDC

SEQ ID NO: 233 - protein, 9 aa, Artificial Sequence
  Peptide (1)..(9): synthetic construct
CVPRRWDVC

SEQ ID NO: 234 - protein, 9 aa, Artificial Sequence
  Peptide (1)..(9): synthetic construct
CQHTSGRGC

SEQ ID NO: 235 - protein, 9 aa, Artificial Sequence
  Peptide (1)..(9): synthetic construct
CRARGWLLC

SEQ ID NO: 236 - protein, 9 aa, Artificial Sequence
  Peptide (1)..(9): synthetic construct
CVSNPRWKC

SEQ ID NO: 237 - protein, 9 aa, Artificial Sequence
  Peptide (1)..(9): synthetic construct
CFNRTWIGC

SEQ ID NO: 238 - protein, 9 aa, Artificial Sequence
  Peptide (1)..(9): synthetic construct
CSRGPAWGC

SEQ ID NO: 239 - protein, 9 aa, Artificial Sequence
  Peptide (1)..(9): synthetic construct
CWSRGQGGC

SEQ ID NO: 240 - protein, 9 aa, Artificial Sequence
  Peptide (1)..(9): synthetic construct
CHVLWSTRC

SEQ ID NO: 241 - protein, 9 aa, Artificial Sequence
  Peptide (1)..(9): synthetic construct
CLGLLMAGC

SEQ ID NO: 242 - protein, 9 aa, Artificial Sequence
  Peptide (1)..(9): synthetic construct
CMSSPGVAC

SEQ ID NO: 243 - protein, 9 aa, Artificial Sequence
  Peptide (1)..(9): synthetic construct
CLASGMDAC

SEQ ID NO: 244 - protein, 9 aa, Artificial Sequence
  Peptide (1)..(9): synthetic construct
CHDERTGRC

SEQ ID NO: 245 - protein, 9 aa, Artificial Sequence
  Peptide (1)..(9): synthetic construct
CAHHALMEC

SEQ ID NO: 246 - protein, 9 aa, Artificial Sequence
  Peptide (1)..(9): synthetic construct
CMQGAATSC

SEQ ID NO: 247 - protein, 9 aa, Artificial Sequence
  Peptide (1)..(9): synthetic construct
CMQGARTSC

SEQ ID NO: 248 - protein, 9 aa, Artificial Sequence
  Peptide (1)..(9): synthetic construct
CVRDLLTGC

SEQ ID NO: 249 - protein, 7 aa, Artificial Sequence
  Peptide (1)..(7): synthetic construct
VPELGHE

SEQ ID NO: 250 - protein, 7 aa, Artificial Sequence
  Peptide (1)..(7): synthetic construct
ELGFELG

SEQ ID NO: 251 - protein, 7 aa, Artificial Sequence
  Peptide (1)..(7): synthetic construct
MGDTGHC

SEQ ID NO: 252 - protein, 7 aa, Artificial Sequence
  Peptide (1)..(7): synthetic construct
LEFNLGY

SEQ ID NO: 253 - protein, 7 aa, Artificial Sequence
  Peptide (1)..(7): synthetic construct
FFLRDWF

SEQ ID NO: 254 - protein, 5 aa, Artificial Sequence
  Peptide (1)..(5): synthetic construct
YRLRG

SEQ ID NO: 255 - protein, 5 aa, Artificial Sequence
  Peptide (1)..(5): synthetic construct
YRARG

SEQ ID NO: 256 - protein, 5 aa, Artificial Sequence
  Peptide (1)..(5): synthetic construct
SQPLG

SEQ ID NO: 257 - protein, 5 aa, Artificial Sequence
  Peptide (1)..(5): synthetic construct
SQPWG

SEQ ID NO: 258 - protein, 6 aa, Artificial Sequence
  Peptide (1)..(6): synthetic construct
QRLVTP

SEQ ID NO: 259 - protein, 6 aa, Artificial Sequence
  Peptide (1)..(6): synthetic construct
QVLVTP

SEQ ID NO: 260 - protein, 6 aa, Artificial Sequence
  Peptide (1)..(6): synthetic construct
QRLVHP

SEQ ID NO: 261 - protein, 6 aa, Artificial Sequence
  Peptide (1)..(6): synthetic construct
QVLVHP

SEQ ID NO: 262 - protein, 7 aa, Artificial Sequence
  Peptide (1)..(7): synthetic construct
ITRWRYL

SEQ ID NO: 263 - protein, 7 aa, Artificial Sequence
  Peptide (1)..(7): synthetic construct
SLGGMSG

SEQ ID NO: 264 - protein, 6 aa, Artificial Sequence
  Peptide (1)..(6): synthetic construct
SQLAAG

SEQ ID NO: 265 - protein, 6 aa, Artificial Sequence
  Peptide (1)..(6): synthetic construct
SQLVAG

SEQ ID NO: 266 - protein, 6 aa, Artificial Sequence
  Peptide (1)..(6): synthetic construct
SLLAAG

SEQ ID NO: 267 - protein, 6 aa, Artificial Sequence
  Peptide (1)..(6): synthetic construct
SLLVAG

SEQ ID NO: 268 - protein, 6 aa, Artificial Sequence
  Peptide (1)..(6): synthetic construct
GLPSGL

SEQ ID NO: 269 - protein, 7 aa, Artificial Sequence
  Peptide (1)..(7): synthetic construct
HGGSANP

SEQ ID NO: 270 - protein, 7 aa, Artificial Sequence
  Peptide (1)..(7): synthetic construct
SLEAFFL

SEQ ID NO: 271 - protein, 9 aa, Artificial Sequence
  Peptide (1)..(9): synthetic construct
CVPELGHEC

SEQ ID NO: 272 - protein, 10 aa, Artificial Sequence
  Peptide (1)..(10): synthetic construct
CFELGFELGC

SEQ ID NO: 273 - protein, 9 aa, Artificial Sequence Sequence
  Peptide (1)..(9): synthetic construct
CGGREFWLC